(19) United States
(12) Patent Application Publication
(10) Pub. No.: US 2023/0285598 A1
(43) Pub. Date: Sep. 14, 2023

(54) SULFOXONIUM YLIDE DERIVATIVES AS PROBES FOR CYSTEINE PROTEASE

(71) Applicant: Takeda Pharmaceutical Company Limited, Osaka (JP)

(72) Inventors: Laura EDGINGTON-MITCHELL, Clayton (AU); Simon MOUNTFORD, Clayton (AU); Bethany M. ANDERSON, Clayton (AU); Monika SZABO, Clayton (AU); Luigi AURELIO, Clayton (AU); Philip THOMPSON, Clayton (AU)

(73) Assignee: Takeda Pharmaceutical Company Limited, Osaka (JP)

(21) Appl. No.: 17/416,180

(22) PCT Filed: Dec. 20, 2019
(86) PCT No.: PCT/JP2019/050228

§ 371 Date: Jun. 18, 2021

(30) Foreign Application Priority Data
Dec. 20, 2018 (AU) 2018904872

(51) Int. Cl. A61K 49/00; C07D 209/30; G01N 33/58

(52) U.S. Cl. CPC A61K 49/0052; C07D 209/30; G01N 33/582; G01N 2333/96466

(57) ABSTRACT

The present invention relates to compounds of formula I bearing a sulfoxoniumylide moiety as warhead, or salts thereof. Such compounds can be used as activity-based probes for cysteine proteases such as cathepsin X, in methods of detecting cysteine protease activity and in related diagnostic or therapeutic methods.

### CROSS REFERENCE TO RELATED APPLICATIONS

This application is a 35 U.S.C. § 371 National Stage filing of International Application No. PCT/JP2019/050228, filed Dec. 20, 2019, which claims the benefit of priority to Australian Patent Application No. 2018904872, filed Dec. 20, 2018. The entire contents of the aforementioned applications are incorporated herein by reference in their entireties.

The present invention relates to compounds that can be used as activity-based probes and/or inhibitors for cysteine proteases such as cathepsin X, methods of detecting cysteine protease activity, and related diagnostic and therapeutic methods and uses.

### BACKGROUND ART

According to Lechtenberg et al., ACS Chem. Biol. 2015, 10, 945-951, “proteases are central mediators of a large variety of physiological processes. Proteolytic cleavage events are at the basis of protein degradation, enzyme activation, and protein maturation and regulate a wide range of pathways from cell death, migration and proliferation, inflammation and immune response, to blood coagulation (Rawlings, N. D., and Salvesen, G. (2012) Handbook of Proteolytic Enzymes, Third edition, Academic Press, Waltham, MA). Aberrant proteolysis on the other hand is frequently linked to serious disorders. Furthermore, proteases are usually expressed in the cell or secreted as inactive zymogens that need activation via processes like proteolytic cleavage or dimerization. Activation of proteases underlies tight temporal and spatial regulation, and thus generally protease location is not an ideal marker for protease function. Instead, spatial-temporal location of the active form of a given protease is necessary for understanding its function. For this purpose, activity-based probes have been developed for a variety of proteases (Deu, E., Verdoes, M., and Bogyo, M. (2012), New approaches for dissecting protease functions to improve probe development and drug discovery. Nat. Struct. Mol. Biol. 19, 9-16.). These probes are designed like active site-reacting protease inhibitors to specfically label an active protease and are thus powerful tools for research and diagnostics. Furthermore, these probes additionally pave the way for the development of potent inhibitors for select proteases for potential therapeutic use (Deu et al. 2012, Nat. Struct. Mol. Biol. 19, 9-16).”

Among the target enzymes of interest are cysteine proteases, including cysteine cathepsins and in particular cathepsin X.

Cysteine cathepsins are a family of lysosomal proteases that are often upregulated in various human cancers, and have been implicated in distinct tumorigenic processes such as angiogenesis, proliferation, apoptosis and invasion. The cysteine cathepsin family constitutes the largest cathepsin family, with 11 proteases in humans referred to as clan CA, family CI a: cathepsins B, C(also known as cathepsin J and dipeptidyl-peptidase 1 ), F, H, K (also known as cathepsin 02), L, O, S, W, V (also known as cathepsin L2), and Z (also known as cathepsin X and cathepsin P). Cathepsins are emerging as major players in tumor progression, making them potential drug targets for a wide range of human cancers.

Cathepsin X (also referred to as cathepsin Z/P) is a cysteine cathepsin protease that is unique among its family members in that it exhibits strict carboxypeptidase activity. Cathepsin X is proposed to underlie many human diseases. Its expression has been associated with several cancer types and neurodegenerative diseases, although its roles during normal physiology are still poorly understood. Advances in our understanding of its function have been hindered by a lack of available tools that can specifically measure the proteolytic activity of cathepsin X.

Cathepsin X contributes to adhesion and maturation of macrophages and dendritic cells (Obermajer et al., 2008) and suppresses clathrin-dependent phagocytosis through cleavage of profiling (Pecar Fonovic and Kos, 2015). Cathepsin X regulates hormone signalling, where its cleavage of bradykinin, kallidin, or angiotensin leads to alterations in specificity towards their cognate receptors and divergent downstream signalling (Nagler et al., 2010). Cathepsin X is also expressed by neurons, where its cleavage of a-enolase regulates survival and the outgrowth of neurites (Obermajer et al., 2009). Furthermore, cathepsin X expression is enriched in amyloid plaques, where it may have a protective effect against neurodegenerative disorders such as Alzheimer’s disease (Hafner et al., 2013; Wendt et al., 2007), and in the spinal cord during neuropathic pain (Leichsenring et al., 2008). Upregulation of cathepsin X mRNA has been reported in pathology-free regions of multiple sclerosis-affected brains (Huynh et al., 2014), and it has been implicated in the generation of IL-lb (Allan et al., 2017; Orlowski et al., 2015) and in mediating neuroinflammation (Allan et al., 2017). It is also upregulated in the microenvironment of breast (Edgington-Mitchell et al., 2015), pancreatic (Akkari et al., 2014), prostate (Nagler et al., 2004), and gastric cancers (Bernhardt et al., 2010; Krueger et al., 2005), where it likely promotes tumor invasion. Thus, cathepsin X holds promise as a clinical biomarker and therapeutic target in diverse diseases.

Like most cathepsins, cathepsin X is synthesized as a zymogen that becomes activated in the acidic environment of endolysosomes. Once activated, it may also be negatively regulated by endogenous inhibitors, though likely not cystatin Cor stefin A (Duivenvoorden et al., 2017; Nagler et al., 1999). In addition to its proteolytic functions, cathepsin X can also promote integrin-mediated signaling through an Arg-Gly-Asp (RGD) motif in its pro-domain (Akkari et al., 2014). As a result of these complex modes of post-translational regulation, traditional biochemical methods that survey total protein levels rarely reflect the pool of proteolytically active enzyme. The ability to specifically measure its activity in its native environment is therefore required to define its precise proteolytic functions during health and disease.

To this end, efforts have been focused on developing fluorescent activity-based probes (ABPs) for cathepsin X. Fluorescent ABPs are small molecules that contain an electrophilic moiety (warhead), a recognition sequence that confers selectivity, and a fluorophore for detection (Edgington and Bogyo, 2013; Edgington et al., 2011; Sannian and Bogyo, 2014). When active, the protease initiates a nucleophilic attack on the warhead, resulting in the formation of a covalent, irreversible bond. Assessment of probe labeling can then be used to quantify protease activity by SDS-PAGE (in-gel fluorescence), fluorescent microscopy, flow cytometry or optical imaging of whole tissues or organisms. Importantly, the covalent nature of probe binding allows for target confirmation by immunoprecipitation with specific antibodies or affinity purification followed by proteomic analysis.

Probes with absolute specificity for cathepsin X have not been previously reported. BMV 109, a fluorescently quenched ABP with a tetrafluorophenoxymethyl ketone warhead, is a pan-cathepsin probe that targets X, B, S, and 10 (Verdoes et al., 2013; Edgington-Mitchell et al., 2015). Because cathepsin X is a similar size to cathepsin B, one of the most abundant and ubiquitously expressed cathepsins, it can be difficult to clearly resolve these two proteases by SDS-PAGE. This precludes accurate quantification by in-gel fluorescence. MGP140 is an epoxide-based activity-based probe that exhibits greater specificity for cathepsin X than BMV109, but also potently reacts with cathepsin B (Paulick and Bogyo, 2011). If mice are pretreated with GB11-NH₂, an inhibitor of cathepsin B, S, and L, prior to MGP140 injection, specific labeling of cathepsin X can be achieved. However, this manipulation of the system results in hyperactivation of cathepsin X, possibly a compensatory response due to the loss of cathepsin B activity. Thus, it is critical to develop activity-based probes with improved specificity for cathepsin X to allow for assessment of its physiological activity.

Shaw discloses the synthesis of peptidyl sulfonium salts and their inhibitory action on the cysteinyl proteases papain and cathepsin B (Shaw, 1988).

Gordon et al., US 5,223,486, disclose peptidyl diazomethyl ketones and peptidyl sulfonium salts as inhibitors of cancer procoagulant, a cysteine protease.

Paulick et al. describe the synthesis and characterization of several fluorescent activity-based probes bearing either an acyloxymethylketone (AOMK), or an epoxide warhead and intended for targeting cathepsin X. It was found that the epoxide-based probes labeled cathepsin X whereas the AOMK probes were uniformly unable to label this protease (Paulick and Bogyo, 2011).

In summary, there remains a need for compounds acting as activity-based probes (or inhibitors) for cysteine proteases such as cysteine cathepsins, and more specifically cathepsin X. Such compounds are useful in investigating the contribution of the respective cysteine protease to normal physiology and disease. Additionally, such compounds can be useful in the diagnosis and/or treatment of diseases associated with the respective cysteine protease activity.

The present inventors attempted to explore new potential warheads for cysteine proteases, and surprisingly found that compounds as described below bearing a sulfoxonium ylide moiety as warhead can act as activity-based probes for cysteine proteases such as cysteine cathepsins, and more specifically cathepsin X, with improved potency and selectivity as compared to previously reported probes (such as BMV109 and MGP140).

### Objects and Summary of the Invention

It is an object of certain embodiments of the present invention to provide activity-based probe compounds that allow detection of cysteine protease activity in biological samples such as cell lysates or tissue lysates.

It is an object of certain embodiments of the present invention to provide activity-based probe compounds that allow detection of cysteine protease activity in live cells.

It is an object of certain embodiments of the present invention to provide activity-based probe compounds that allow detection of cysteine protease activity in vivo.

It is an object of certain embodiments of the present invention to provide activity-based probe compounds that allow detection of cathepsin X activity in biological samples such as cell lysates or tissue lysates.

It is an object of certain embodiments of the present invention to provide activity-based probe compounds that allow detection of cathepsin X activity in live cells.

It is an object of certain embodiments of the present invention to provide activity-based probe compounds that allow detection of cathepsin X activity in vivo.

It is an object of certain embodiments of the present invention to provide activity-based probe compounds that allow detection of cysteine protease activity with improved selectivity as compared to the activity-based probes BMV109 and MGP140.

It is an object of certain embodiments of the present invention to provide activity-based probe compounds that allow detection of cathepsin X activity with improved selectivity as compared to the activity-based probes BMV109 and MGP140.

It is an object of certain embodiments of the present invention to provide activity-based probe compounds that allow detection of cathepsin X activity with high potency for cathepsin X, such as improved potency for cathepsin X as compared to the activity-based probes BMV109 and MGP140.

It is an object of certain embodiments of the present invention to provide activity-based probe compounds that allow detection of cathepsin X activity (e.g., in cell lysates, tissue lysates, live cells and in vivo) and that exhibit no cross-reactivity with cathepsin B and/or cathepsin L.

It is an object of certain embodiments of the present invention to provide inhibitors of a cysteine protease.

It is an object of certain embodiments of the present invention to provide inhibitors of cathepsin X.

It is an object of certain embodiments of the present invention to provide inhibitors of cathepsin X that exhibit no cross-reactivity with cathepsin B and/or cathepsin L.

It is an object of certain embodiments of the present invention to provide compounds that can be used in the diagnosis of a disease associated with a cysteine protease activity.

It is an object of certain embodiments of the present invention to provide compounds that can be used in the diagnosis of a disease associated with cathepsin X activity.

It is an object of certain embodiments of the present invention to provide compounds that can be used in the diagnosis of a disease selected from the group consisting of cancer, inflammatory diseases and neurodegenerative diseases.

It is an object of certain embodiments of the present invention to provide compounds that can be used in the treatment of a disease associated with a cysteine protease activity.

It is an object of certain embodiments of the present invention to provide compounds that can be used in the treatment of a disease associated with cathepsin X activity.

It is an object of certain embodiments of the present invention to provide compounds that can be used in the treatment of a disease selected from the group consisting of cancer, inflammatory diseases and neurodegenerative diseases.

The above objects are to be understood to also relate to the respective methods as well as to compounds/compositions for use in the respective method.

In certain embodiments, the present invention is directed to a compound of formula I

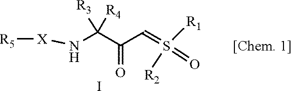

or a salt thereof, wherein
R₁is selected from the group consisting of (C₁-C₈)alkyl, (C₁-C₈)hydroxyalkyl, (C₁-C₈) haloalkyl, (C₃-C₈) cycloalkyl, (C₂-C₈) alkenyl and (C₂-C₈) alkynyl;
R₂ is selected from the group consisting of (C₁-C₈)alkyl, (C₁-C₈)hydroxyalkyl, (C₁-C₈) haloalkyl, (C₃-C₈) cycloalkyl, (C₁-C₈)alkenyl and (C₂-C₈) alkynyl;
R₃ is the sidechain of an alpha amino acid;
R₄ is selected from the group consisting of hydrogen and (C₁-C₄) alkyl;
R₅ is selected from the group consisting of a detectable element, an amine protecting group, (C₁-C₈)alkyl, (C₁-C₈)hydroxyalkyl, (C₁-C₈)haloalkyl, (C₃-C₈) cycloalkyl, (C₂-C₈) alkenyl, (C₂-C₈) alkynyl, (C₁-C₈) alkylcarbonyl, (C₁-C₈)hydroxyalkylcarbonyl, (C₁-C₈) haloalkylcarbonyl, (C₃-C₈) cycloalkylcarbonyl, (C₁-C₈)alkyloxycarbonyl, benzyloxycarbonyl, and hydrogen;
X is
   (i) a bond; or
   (ii) a biradical moiety of formula II or III which is connected to the R₅ substituent via the amino group

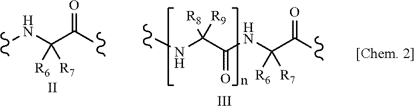

wherein
R₆ is the sidechain of an alpha amino acid;
R₇ is selected from the group consisting of hydrogen and (C₁-C₄) alkyl;
R₈ is the sidechain of an alpha amino acid;
R₉ is selected from the group consisting of hydrogen and (C₁-C₄) alkyl; and n is 1, 2, 3, or 4.

In certain embodiments, the present invention is directed to a compound (of formula I) having a formula selected from the following group of formulas:

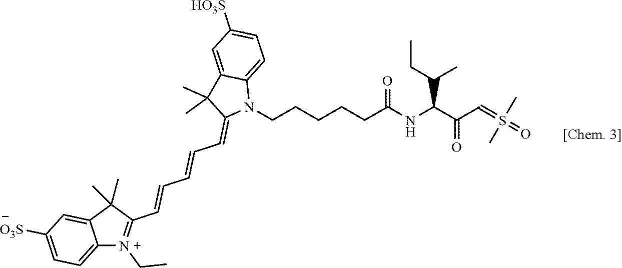

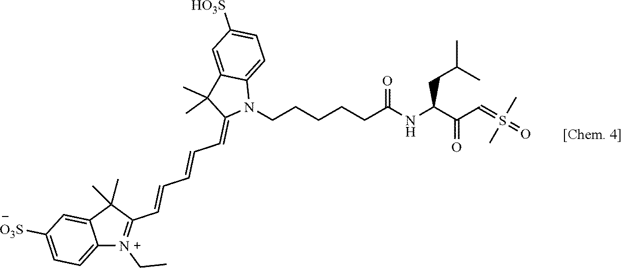

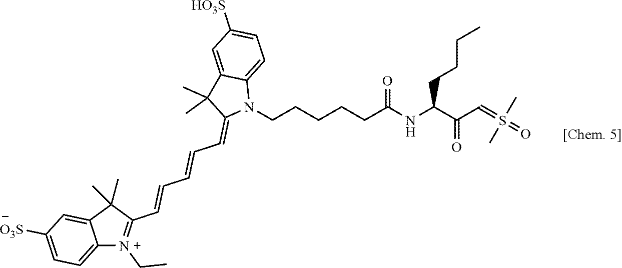

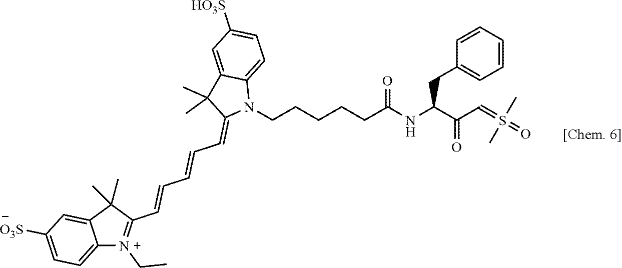

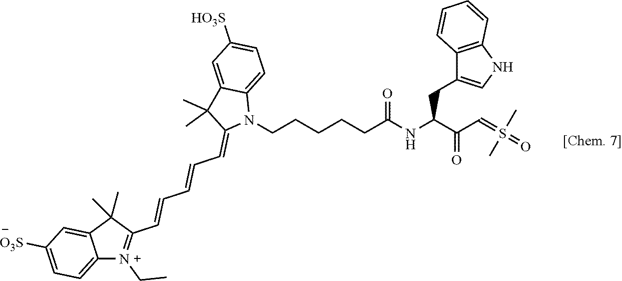

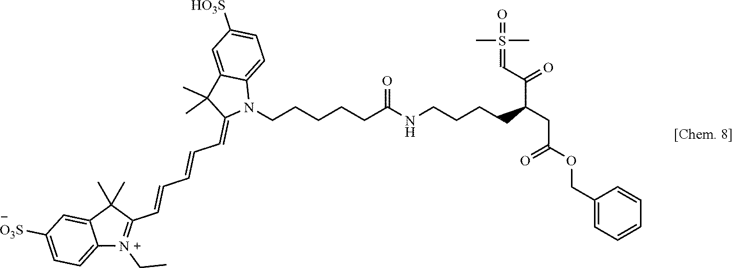

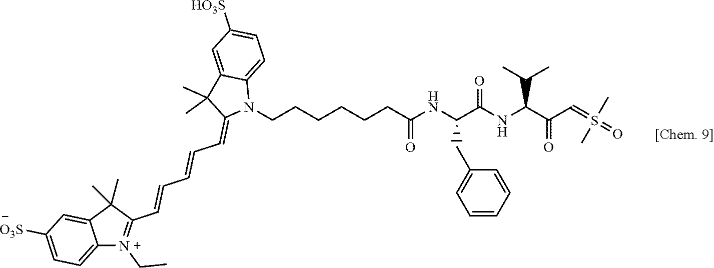

or a salt thereof.

In certain embodiments, the present invention is directed to a compound (of formula I) having the following formula:

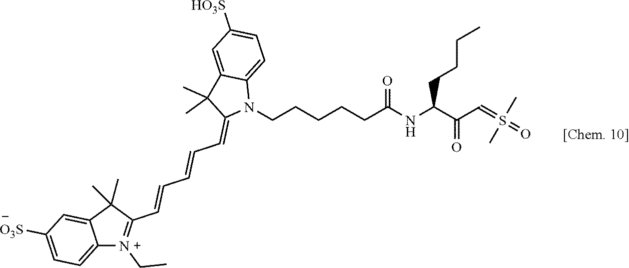

or a salt thereof.

In certain embodiments, the present invention is directed to a composition comprising a compound of formula I as described herein or a salt thereof, and an excipient.

In certain embodiments, the present invention is directed to a composition comprising a compound of formula I as described herein or a salt thereof, and an excipient, wherein the compound comprises at least one detectable element.

In certain embodiments, the present invention is directed to a method of detecting cysteine protease activity comprising
(1) contacting the cysteine protease with an activity-based probe compound comprising a sulfoxonium ylide moiety as warhead, and
(2) subsequently analyzing the cysteine protease comprising measuring a detectable signal.

In certain embodiments, the present invention is directed to an in vitro method of detecting cysteine protease activity comprising
(1) contacting a biological sample with an activity-based probe compound comprising a sulfoxonium ylide moiety as warhead, and
(2) subsequently analyzing the biological sample comprising measuring a detectable signal.

In certain embodiments, the present invention is directed to a method of detecting cysteine protease activity in a biological sample obtained from a subject comprising
(1) contacting the biological sample in vitro with an activity-based probe compound comprising a sulfoxonium ylide moiety as warhead, and
(2) subsequently analyzing the biological sample comprising measuring a detectable signal.

In certain embodiments, the present invention is directed to a method of detecting cysteine protease activity comprising
(1) contacting the cysteine protease with a compound of formula I as described herein or a salt thereof, or with a composition as described herein, and
(2) subsequently analyzing the cysteine protease comprising measuring a detectable signal.

In certain embodiments, the present invention is directed to an in vitro method of detecting cysteine protease activity comprising
(1) contacting a biological sample with a compound of formula I as described herein or a salt thereof, or with a composition as described herein, and
(2) subsequently analyzing the biological sample comprising measuring a detectable signal.

In certain embodiments, the present invention is directed to a method of detecting cysteine protease activity in a biological sample obtained from a subject comprising
(1) contacting the biological sample in vitro with a compound of formula I as described herein or a salt thereof, or with a composition as described herein, and
(2) subsequently analyzing the biological sample comprising measuring a detectable signal.

In certain embodiments, the present invention is directed to a method of detecting cysteine protease activity comprising
(1) administering to a subject an activity-based probe compound comprising a sulfoxonium ylide moiety as warhead,
(2) subsequently obtaining a biological sample from the subject; and
(3) subsequently analyzing the biological sample comprising measuring a detectable signal.

In certain embodiments, the present invention is directed to a method of detecting cysteine protease activity comprising
(1) administering to a subject a compound of formula I as described herein or a salt thereof, or a composition as described herein,
(2) subsequently obtaining a biological sample from the subject; and
(3) subsequently analyzing the biological sample comprising measuring a detectable signal.

In certain embodiments, the present invention is directed to an in vivo method of detecting cysteine protease activity in a subject comprising
(1) administering to the subject an activity-based probe compound comprising a sulfoxonium ylide moiety as warhead, and
(2) subsequently examining the subject comprising measuring a detectable signal.

In certain embodiments, the present invention is directed to an in vivo method of detecting cysteine protease activity in a subject comprising
(1) administering to the subject a compound of formula I as described herein or a salt thereof, or a composition as described herein, and
(2) subsequently examining the subject comprising measuring a detectable signal.

In certain embodiments, the present invention is directed to a method of inhibiting a cysteine protease comprising contacting the cysteine protease with a compound of formula I as described herein or a salt thereof, or with a composition as described herein.

In certain embodiments, the present invention is directed to an in vitro method of inhibiting a cysteine protease comprising contacting a biological sample with a compound of formula I as described herein or a salt thereof, or with a composition as described herein.

In certain embodiments, the present invention is directed to a method of inhibiting a cysteine protease in a biological sample obtained from a subject comprising contacting the biological sample in vitro with a compound of formula I as described herein or a salt thereof, or with a composition as described herein.

In certain embodiments, the present invention is directed to an in vivo method of inhibiting a cysteine protease in a subject comprising administering to the subject a compound of formula I as described herein or a salt thereof, or a composition as described herein.

In certain embodiments, the present invention is directed to a method of diagnosing a disease associated with a cysteine protease activity in a subject comprising
(1) contacting a biological sample obtained from the subject in vitro with a compound of formula I as described herein or a salt thereof, or with a composition as described herein, and
(2) subsequently analyzing the biological sample comprising measuring a detectable signal.

In certain embodiments, the present invention is directed to a method of diagnosing a disease associated with a cysteine protease activity in a subject comprising
(1) administering to the subject a compound of formula I as described herein or a salt thereof, or a composition as described herein,
(2) subsequently obtaining a biological sample from the subject; and
(3) subsequently analyzing the biological sample comprising measuring a detectable signal.

In certain embodiments, the present invention is directed to an in vivo method of diagnosing a disease associated with a cysteine protease activity in a subject comprising
(1) administering to the subject a compound of formula I as described herein or a salt thereof, or a composition as described herein, and
(2) subsequently examining the subject comprising measuring a detectable signal.

In certain embodiments, the present invention is directed to a compound of formula I as described herein or a salt thereof, for use in a method of diagnosing a disease associated with a cysteine protease activity in a subject, wherein the method comprises
(1) administering to the subject a compound of formula I as described herein or a salt thereof,
(2) subsequently obtaining a biological sample from the subject; and
(3) subsequently analyzing the biological sample comprising measuring a detectable signal.

In certain embodiments, the present invention is directed to a composition as described herein for use in a method of diagnosing a disease associated with a cysteine protease activity in a subject, wherein the method comprises
(1) administering to the subject a composition as described herein,
(2) subsequently obtaining a biological sample from the subject; and
(3) subsequently analyzing the biological sample comprising measuring a detectable signal.

In certain embodiments, the present invention is directed to a compound of formula I as described herein or a salt thereof, for use in an in vivo method of diagnosing a disease associated with a cysteine protease activity in a subject, wherein the method comprises
(1) administering to the subject a compound of formula I as described herein or a salt thereof, and
(2) subsequently examining the subject comprising measuring a detectable signal.

In certain embodiments, the present invention is directed to a composition as described herein for use in an in vivo method of diagnosing a disease associated with a cysteine protease activity in a subject wherein the method comprises
(1) administering to the subject a composition as described herein, and
(2) subsequently examining the subject comprising measuring a detectable signal.

In certain embodiments, the present invention is directed to a method of treating a disease associated with a cysteine protease activity comprising administering to a patient in need thereof a therapeutically effective amount of a compound of formula I as described herein or a salt thereof, or a therapeutically effective amount of a composition as described herein.

In certain embodiments, the present invention is directed to a compound of formula I as described herein or a salt thereof for use in the treatment of a disease associated with a cysteine protease activity.

In certain embodiments, the present invention is directed to a composition for use in the treatment of a disease associated with a cysteine protease activity comprising a compound of formula I as described herein or a salt thereof and a pharmaceutically acceptable excipient.

In certain embodiments, the present invention is directed to a use of a compound of formula I as described herein or a salt thereof in the manufacture of a medicament for the treatment of a disease associated with a cysteine protease activity.

In certain embodiments, the present invention is directed to a use of a composition as described herein in the manufacture of a medicament for the treatment of a disease associated with a cysteine protease activity.

In certain embodiments, the present invention is directed to a process for preparing a compound bearing a chloromethylketone moiety comprising reacting a compound bearing a sulfoxonium ylide moiety to yield the compound bearing the chloromethylketone moiety.

In certain embodiments, the present invention is directed to a process for preparing an activity-based probe compound bearing an acyloxymethylketone moiety as warhead, comprising
(i) preparing an intermediate compound bearing a chloromethylketone moiety by reacting a compound bearing a sulfoxonium ylide moiety to yield the compound bearing the chloromethylketone moiety; and
(ii) further processing the compound bearing the chloromethylketone moiety to yield said activity-based probe compound.

### BRIEF DESCRIPTION OF DRAWINGS

FIG. 1A depicts the structure of the sCy5-Val-SY probe.

FIG. 1B depicts a proposed mechanism of binding of the sCy5-Val-SY probe to a cysteine protease.

FIG. 2A depicts results of Example 3 (Labeling of RAW264.7 lysates with sCy5-Val-SY (at 1 µM for 20 minutes) or BMV109 (at 1 µM for 20 minutes) alone or after pretreatment with 10 µM MDV-590 (cathepsin S inhibitor) or JPM-OEt (pan cysteine cathepsin inhibitor).

FIG. 2A-1 depicts results of Example 3 (Labeling of RAW264.7 lysates with the indicated concentrations of sCy5-Val-SY for 20 minutes, as analyzed by in-gel fluorescence).

FIG. 2A-2 depicts results of Example 3 (Labeling of RAW264.7 lysates with 1 µM sCy5-Val-SY for the indicated time, as analyzed by in-gel fluorescence).

FIG. 2B depicts results of Example 3 (Immunoprecipitation of sCy5-Val-SY-labeled samples (labeling at 1 µM for 20 minutes) with a cathepsin X-specific antibody).

FIG. 2C depicts the results of Example 4 (Labeling of splenic lysates from wildtype or cathepsin X-deficient mice with sCy5-Val-SY or BMV109).

FIG. 2D depicts results of Example 5 (Labeling of living RAW264.7 cells with increasing doses of sCy5-Val-SY or BMV 109 for two hours).

FIG. 2D-1 depicts results of Example 5 (Labeling of live (intact) RAW264.7 cells with 1 µM sCy5-Val-SY for the indicated time, analyzed by in-gel fluorescence).

FIG. 2E depicts results of Example 5 (Immunoprecipitation of sCy5-Val-SY-labeled samples (live-cell labeling at 1 µM for 2 hours) with a cathepsin X-specific antibody).

FIG. 2F depicts results of Example 5 (Labeling of living RAW264.7 cells, with and without overnight pretreatment with 10 µM MDV-590, with sCy5-Val-SY or BMV109 (1 µM, two hours).

[FIG. 3A] FIG. 3A depicts the results of Example 6 (Labeling of RAW264.7 lysates with the indicated sulfoxonium ylide probe or BMV109 (probe added at increasing concentrations of 0.01, 0.05, 0.1, 0.5, 1, 5 µM, as indicated by the arrow of increasing thickness)).

FIG. 3B depicts the results of Example 7 (Labeling of kidney lysates with the indicated sulfoxonium ylide probe or BMV109 (probe added at increasing concentrations of 0.1, 0.5, 1, 5 µM, as indicated by the arrow of increasing thickness)).

FIG. 3C depicts the results of Example 8 (Labeling of live RAW264.7 cells with the indicated sulfoxonium ylide probe or BMV 109 (probe added at increasing concentrations of 0.1, 0.5, 1, 5 µM, as indicated by the arrow of increasing thickness)).

FIG. 3D depicts the results of Example 9 (Labeling of live MDA-MB-231 ^{HM} cells with the indicated sulfoxonium ylide probe or BMV109 (probe added at increasing concentrations of 0.1, 0.5, 1, 5 µM, as indicated by the arrow of increasing thickness)).

FIG. 4A depicts results of Example 10 (SDS-PAGE and in-gel fluorescence of tissues from mice that received no probe (NP), sCy5-Nle-SY, or BMV109. BMV109-labeled samples are cut from the same gel and are presented at the same gain setting as the other samples in the corresponding tissue. Gains for each tissue were set individually to display optimal contrast for cathepsin X labeling. An autofluorescent band was observed in the no-probe control.

FIG. 4B depicts results of Example 10 (Immunoprecipitation of liver and kidney samples with a cathepsin X-specific antibody).

FIG. 5 depicts results of Example 10 (Confocal microscopy of cathepsin X labeling in kidney with sCy5-Nle-SY. Kidney sections from sCy5-Nle-SY-injected mice or no-probe control were analyzed for sCy5 fluorescence or cathepsin X immunoreactivity.

FIG. 6A depicts the results of Example 11 (Labeling of living RAW264.7 cells with the indicated AOMK and sulfoxonium ylide probes (at increasing concentrations of 0.1, 0.5, 1, and 5 µM, as indicated by the arrow of increasing thickness), as analyzed by in-gel fluorescence. In the top panel, gain settings are equal for all samples. In the bottom panel, gain settings were individually set to show optimal contrast for the AOMK probes.

FIG. 6B depicts the results of Example 12 (In vivo labeling of colons with the sCy5-Nle-AOMK probe. The top band is an autofluorescent protein that appears in no-probe controls).

FIG. 7 depicts the results of experiments relating to the identification of probe-labeled proteases. (A,C-G): Immunoprecipitations of probe-labelled lysates (RAW264.7 cells or kidney, with the indicated probe, administered to live cells or lysates as indicated) with cathepsin-specific antibodies. (B) Labeling of living RAW264.7 cells, with and without overnight pretreatment with cathepsin S inhibitors MDV590 (10 µM) or Z-FL (20 µM), with sCy5-Nle-SY (1 µM, two hours).

FIG. 8A depicts the results of Example 13 (labeling of lysates prepared from human oral squamous cell carcinoma tissue or patient-matched normal oral mucosa with sCy5-Nle-SY (incubation at 5 µM for 20 minutes) and analysis by in-gel fluorescence, as well as immunoblot of the samples).

FIG. 8B depicts the results of Example 13 (Immunoprecipitation of Cy5-Nle-SYlabeled human oral cancer tissue lysates with a cathepsin X-specific antibody).

FIG. 9 depicts the results of Example 14 (labeling of RAW264.7 cells which were pre-treated with Boc-Val-SY (0, 10 or 100 µM) overnight with sCy5-Val-SY, and analysis of lysed cells by in-gel fluorescence).

### DETAILED DESCRIPTION

In describing the present invention, the following terms are to be used as indicated below.

As used herein, the singular forms “a”, “an”, and “the” include plural references unless the context clearly indicates otherwise.

Unless defined otherwise, all technical and scientific terms used herein have the same meanings as commonly understood by one of ordinary skill in the art.

The term “activity-based probe” is intended to have the same meaning as commonly understood by one of ordinary skill in the art. Activity-based probes (ABPs) are small molecules that covalently bind to the active site of an enzyme (such as a protease) or a group of enzymes in an activity-dependent manner (i.e., the labeling reaction requires enzyme activity). ABPs typically include three elements: (i) an electrophilic moiety called “warhead”, (ii) a linker or recognition sequence, and (iii) a detectable element or “reporter moiety” for detection. The enzyme attacks the electrophilic warhead resulting in the formation of a covalent adduct which can then be detected either directly (e.g., if the detectable element is a fluorescent label), or by two-step labeling (e.g., post-labeling modification of a ligation handle).

The term “detectable element” or “reporter group/moiety” refers to a functional group in a compound (activity-based probe) that can be detected using techniques including, but not limited to, optical methods (e.g., measurement of fluorescence or UV-VIS absorbance), radiography, biochemical methods (e.g., using an immunochemical reagent such as an antibody), etc. The term “detectable element” includes functional groups that can be detected “directly” (e.g., by fluorescence measurement after running an SDS-PAGE) as well as functional groups that can be detected after performing a secondary labeling step and subsequent detection of the secondary label. An example for such groups is a biotin label which can be detected, e.g., after secondary labeling with fluorescently tagged streptavidin and subsequent fluorescence measurement. A further example for such groups is a click-chemistry label (bioorthogonal ligation handle) which can be detected, e.g., after secondary labeling with a fluorescent label using a click-chemistry (bioorthogonal) reaction and subsequent fluorescence measurement.

A “bioorthogonal ligation handle” is thus a functional group present in the compounds of the invention at the initial probe labeling step (in vivo or ex vivo contacting of the protease/biological sample/subject with the compounds of the invention), which enables the subsequent attachment of a secondary label (corresponding to the actually detected label) in a secondary labeling step using e.g. a click-chemistry (bioorthogonal) reaction which is performed in vitro.

Click-chemistry labels and respective click-chemistry reactions for secondary labeling, i.e., attachment of the label to be actually detected, are described, e.g., in Martell et al., Applications of Copper-Catalyzed Click Chemistry in Activity-Based Protein Profiling, Molecules 2014, 19, 1378-1393, and in Willems et al., Bioorthogonal chemistry: Applications in activity-based protein profiling, Acc. Chem. Res. 2011, 44, 718-729.

The term “cysteine protease activity” refers to proteolytic activity of the cysteine protease. In the case of cathepsin X, the term “cysteine protease activity” or “cathepsin X activity” refers to proteolytic activity which is carboxypeptidase activity.

Cathepsin X, S, B and L each belong to the cysteine protease family. The term “cathepsin X” denotes a protein resulting from the CTSZ gene locus. “Cathepsin X” is also referred to as “cathepsin Z” or “cathepsin P”. The term “cathepsin S” denotes a protein resulting from the CTSS gene locus. The term “cathepsin B” denotes a protein resulting from the CTSB gene locus. The term “cathepsin L” denotes a protein resulting from the CTSL gene locus.

The term “patient” means a subject, particularly a human, who has presented a clinical manifestation of a particular symptom or symptoms suggesting the need for treatment, who is treated preventatively or prophylactically for a condition, or who has been diagnosed with a condition to be treated.

The term “subject” is meant to comprise mammalian subjects, in particular human subjects, and is inclusive of the definition of the term “patient” and does not exclude individuals who are entirely normal in all respects or with respect to a particular condition.

As used herein, the term “therapeutically effective” refers to the amount of drug or the rate of drug administration needed to produce a desired therapeutic result.

As used herein, terms like “administration” or ”administering” include various administration routes, such as intravenously, subcutaneously, intramuscularly, orally, nasally, sublingually, or topically.

The term “tissue sample” or “tissue biopsy” refers to a sample of a biological tissue obtained from a subject, such as a sample obtained by excision, needle aspiration, biopsy forceps, or swab. Tissue samples also comprise mucosal biopsies and fecal samples. The sampled tissue can be live, dead, healthy, or diseased and contain a heterogenous mixture of cell types and extracellular factors.

A “mucosal biopsy” is typically obtained by swabbing mucus accumulated on the surface of another tissue, e.g. mucous membranes or intestinal tract epithelia. Mucosal biopsies contain shed cells and cell excretions from the tissue the mucus accumulated on.

The term “sputum sample” refers to a sample that is a mixture of saliva and mucus coughed up from the respiratory tract. A “sputum sample” can be obtained invasively or non-invasively. Invasive methods involve oropharyngeal or endotracheal suctioning while the subject is intubated, and the obtained contents are collected in a sputum trap. Non-invasive methods collect the contents produced when the subject coughs, sometimes after nebulization with saline to loosen secretions.

The term “fecal sample” or “stool sample” refers to a sample collected from the feces of a subject. Fecal samples comprise cells shed from the gastrointestinal tract and cell excretions from the gastrointestinal tract of the subject.

The term “tissue sample lysate” refers to a solution obtained by lysing the cells of a tissue sample. The term “lysing” or “lysis” refers to the disintegration or rupture of the cell membranes, resulting in the release of cell contents and/or the subsequent death of the cell. Lysis can be accomplished e.g. by mechanical, enzymatic, or osmotic disruption of the cell membranes.

The term “disease associated with a cysteine protease activity” as used herein denotes a disease wherein a cysteine protease activity is implicated in the pathogenesis of the disease. In a “disease associated with a cysteine protease activity”, the level of cysteine protease activity in the diseased state or diseased region of the body (e.g., body part, organ, pathological tissue including tumor tissue), deviates from the respective level of cysteine protease activity found in the pathology-free state or in the respective pathology-free region of the body. In certain embodiments, the level of cysteine protease activity in the diseased state or diseased region of the body, is increased as compared to the respective level of cysteine protease activity found in the pathology-free state or in the respective pathology-free region of the body. For example, in the pathology-free state or region, the level of cysteine protease activity can be below a detectable limit, whereas in the diseased state or region, the level of cysteine protease activity is above the detectable limit. Diseases associated with a cysteine protease activity include celiac disease, gastrointestinal motility disorders, pain, itch, skin disorders (such as topic dermatitis), diet-induced obesity, metabolic disorders (including, but not limited to nonalcoholic steatohepatitis (NASH), hepatic and pancreatic disease), asthma, rheumatoid arthritis, periodontitis, inflammatory diseases (such as inflammatory GI disorders, in particular inflammatory bowel diseases), functional GI disorders (such as irritable bowel syndrome, functional chest pain, functional dyspepsia, nausea and vomiting disorders, functional constipation, functional diarrhea, fecal incontinence, functional anorectal pain, and functional defecation disorders), cancer, fibrotic diseases, metabolic dysfunctions, neurological diseases, and neurodegenerative diseases. In certain embodiments the “disease associated with a cysteine protease activityis a “disease associated with cathepsin X activity”.

The term “inflammatory gastrointestinal disease”, “inflammatory gastrointestinal disorder”, “inflammatory GI disorder”, or “inflammatory GI disease” as used herein denotes gastrointestinal diseases, i.e. diseases involving the gastrointestinal tract, namely the oral cavity, esophagus, stomach, small intestine, large intestine (colon) and rectum, and the accessory organs of digestion (e.g., the tongue, salivary glands, pancreas, liver and gallbladder), in which there is inflammation of one or more parts of the GI tract. Inflammatory GI diseases comprise, e.g., inflammatory bowel diseases, infectious diarrhea, mesenteric ischemia, diverticulitis, and necrotizing enterocolitis (NEC).

The term “inflammatory bowel disease” or “IBD” refers to a collection of diseases characterized by chronic and relapsing inflammation in the gastrointestinal tract. IBD most notably comprises ulcerative colitis (UC) and Crohn’s disease (CD), both of which are associated with diarrhea, rectal bleeding, increased urgency, and pain, but also comprises less prevalent diseases such as acute colitis, immuno-oncology colitis, chemotherapy/radiation colitis, Graft versus Host Disease (GvHD) colitis, collagenous colitis, lymphocytic colitis, microscopic colitis, diversion colitis, Beh&ccedil;et’s disease, and indeterminate colitis and pouchitis.

The term “functional gastrointestinal disorders”, “functional GI disorders” or “functional GI diseases” as used herein denotes disorders of gut-brain interaction. It is a group of disorders classified by GI symptoms related to any combination of the following: motility disturbance, visceral hypersensitivity, altered mucosal and immune function, altered gut microbiota, and altered central nervous system (CNS) processing. The term “functional” is generally applied to disorders in which the body’s normal activities in terms of the movement of the intestines, the sensitivity of the nerves of the intestines, or the way in which the brain controls some of these functions is impaired. However, there are no structural abnormalities that can be seen by endoscopy, x-ray, or blood tests. Thus, these disorders are largely identified by the characteristics of the symptoms. Functional GI disorders comprise irritable bowel syndrome, functional chest pain, functional dyspepsia, nausea and vomiting disorders, functional constipation, functional diarrhea, fecal incontinence, functional anorectal pain, and functional defecation disorders.

The term “infection” refers to a process or state wherein an infectious agent (such as, e.g., pathogenic bacteria, fungi, protozoa, viruses, prions, viroids, nematodes, and helminths) invade and multiply in the body tissues of an infected subject.

The term “cancer” refers to a collection of diseases characterized by uncontrolled, abnormal growth of cells with the potential to invade or spread to other parts of the body. Cancer can affect any tissue and is named after the tissue of origin. The term “oral cancer” refers to cancers of the mouth, i.e. any cancerous tissue growth located in the oral cavity of a subject. Exemplary histological types of oral cancer are teratoma, adenocarcinoma derived from a major or minor salivary gland, lymphoma from tonsillar or other lymphoid tissue, or melanoma from the pigment-producing cells of the oral mucosa. The most common type of oral cancer is squamous cell carcinoma originating in the tissues that line the mouth and lips, with less common types including Kaposi’s sarcoma. Oral cancer most commonly involves the tongue, but may also occur on the floor of the mouth, cheek lining, gingiva, lips, or palate. The term “breast cancer” refers to cancers of the breast. Exemplary breast cancers are ductal carcinoma in situ (DCIS), lobular carcinoma in situ (LCIS), invasive ductal carcinoma (IDC), invasive lobular carcinoma (ILC), Paget disease of the nipple, phyllodes tumor, and angiosarcoma. The term “prostate cancer” refers to cancer of the prostate. Exemplary prostate cancers include adenocarcinomas of the prostate. The term “colorectal cancer” refers to cancers of the colon and/or rectum. Exemplary colorectal cancers are adenocarcinomas, carcinoid tumors, gastrointestinal stromal tumors (GISTs), lymphomas, and sarcomas originating from the colon or rectum.

The term “alpha amino acid” is meant to comprise natural and unnatural alpha amino acids.

The term “natural amino acid” is meant to comprise proteinogenic and non-proteinogenic amino acids.

With respect to sidechains of alpha amino acids, the term “structural analog” refers to a sidechain wherein a CH₂ group is replaced by O, S, or NH, and/or wherein a —CH₂—CH₂—group is replaced by a —CH═CH—group.

With respect to sidechains of alpha amino acids, the term “homologue” refers to a sidechain which is extended by one CH₂ group.

The term “(C_{y}-C_{z})” when used in conjunction with a chemical group, such as alkyl, alkenyl, alkynyl, cycloalkyl, alkoxy, aryl etc., indicates the possible number of carbon atoms in the group (i.e., from y to z carbon atoms).

The term “alkyl” as used herein denotes a straight-chain or branched alkyl group. Examples of alkyl groups include methyl, ethyl, n-propyl, iso-propyl, n-butyl, 2-butyl, iso-butyl, tert-butyl, n-pentyl, 1-methylbutyl, 2-methylbutyl, 3-methylbutyl, and 2,2-dimethylpropyl.

The term “haloalkyl” as used herein denotes a straight-chain or branched alkyl group, wherein the hydrogen atoms of this group are partially or totally replaced with halogen atoms. Examples of haloalkyl groups include fluoromethyl, difluoromethyl, trifluoromethyl, 1-fluoroethyl, 2-fluoroethyl, 2,2-difluoroethyl, 2,2,2-trifluoroethyl, and the like. In one embodiment, from 1 to 3 hydrogen atoms are replaced with halogen atoms.

The term “hydroxyalkyl” as used herein denotes a straight-chain or branched alkyl group, wherein at least one hydrogen atom of this group is replaced with a hydroxy group. In certain embodiments one or two hydrogen atoms are replaced with a hydroxy group. In certain embodiments one hydrogen atom is replaced with a hydroxy group.

The term “cycloalkyl” as used herein denotes a saturated monocyclic hydrocarbon radical. Examples of cycloalkyl groups include cyclopropyl, cyclobutyl, cyclopentyl, cyclohexyl, cycloheptyl, cyclooctyl, cyclononyl and cyclodecyl.

The term “alkenyl” as used herein denotes an at least singly unsaturated, straight-chain or branched hydrocarbon radical, i.e. a straight-chain or branched hydrocarbon radical having at least one carbon-carbon double bond. Examples of alkenyl groups include, e.g. vinyl, allyl (2-propen-1-yl), 1-propen-1-yl, 2-propen-2-yl, methallyl (2-methylprop-2-en-1-yl), 2-buten-1-yl, 3-buten-1-yl, 2-penten-1-yl, 3-penten-1-yl, 4-penten-1-yl, 1-methylbut-2-en-1-yl, 2-ethylprop-2-en-1-yl and the like.

The term “alkynyl” as used herein denotes a straight-chain or branched hydrocarbon radical having at least one carbon-carbon triple bond. Examples of alkynyl groups include ethynyl, propargyl (2-propyn-1-yl, also referred to as prop-2-yn-1-yl), 1-propyn-1-yl (also referred to as prop-1-yn-1-yl), 1-methylprop-2-yn-1-yl, 2-butyn-1-yl, 3-butyn-1-yl, 1-pentyn-1-yl, 3-pentyn-1-yl, 4-pentyn-1-yl, 1-methylbut-2-yn-1-yl, 1-ethylprop-2-yn-1-yl and the like.

The term “aryl” as used herein denotes groups derived from monocyclic or polycyclic aromatic hydrocarbons by removal of a hydrogen atom from a ring carbon atom. Examples of aryl groups include phenyl and naphtyl.

The term “heteroaryl” as used herein denotes groups derived from heteroarenes by removal of a hydrogen atom from any ring atom. Examples of heteroaryl groups include groups derived from pyrrole, furan, thiophene, imidazole, oxazole, thiazole, pyrazole, pyridine, pyrazine, pyridazine, pyrimidine, indole, and the like. Typical heteroatoms of heteroarenes are nitrogen, oxygen and sulfur.

The term “halo” or “halogen” as used herein denotes fluorine, bromine, chlorine or iodine, in particular fluorine, or chlorine.

The term “alkoxy” as used herein denotes a straight-chain or branched alkyl group, which is bonded via an oxygen group to the remainder of the molecule. Examples of alkoxy groups include methoxy, ethoxy, n-propoxy, iso-propoxy, n-butyloxy, 2-butyloxy, iso-butyloxy, tert-butyloxy, and the like.

The term “alkylcarbonyl” refers to a straight-chain or branched alkyl group, which is bonded via the carbon atom of a carbonyl group (C═O) to the remainder of the molecule.

The term “amine protecting group” refers to a chemical moiety that renders an amino group unreactive, but is also removable so as to restore the amino group. Examples of amine protecting groups include benzyloxycarbonyl (Cbz), 9-fluorenylmethyloxycarbonyl (Fmoc), tert-butyloxycarbonyl (Boc), allyloxycarbonyl (Alloc), p-toluene sulfonyl (Tos), 2,2,5,7,8-pentamethylchroman-6-sulfonyl (Pmc), 2,2,4,6,7-pentamethyl-2,3-dihydrobenzofuran-5-sulfonyl (Pbf), mesityl-2-sulfonyl (Mts), 4-methoxy-2,3,6-trimethylphenylsulfonyl (Mtr), acetamido, phthalimido, and the like. Other amine protecting groups are known to those of skill in the art including, for example, those described by Green and Wuts (Protective Groups in Organic Synthesis, 4 ^{th} Ed. 2007, Wiley-Interscience, New York) and by P. Kocienski (Thieme, 2005).

The term “(C₆-C₁₀) arylmethyl” as used herein denotes a methyl group substituted by a (C₆-C₁₀) aryl group. An example of a (C₆-(C₁₀) arylmethyl group is benzyl.

The term “(C₃-C₉) heteroarylmethyl” as used herein denotes a methyl group substituted by a (C₃-C₉) heteroarylmethyl group. An example of a (C₃-C₉) heteroarylmethyl group is (1H-indol-3-yl) methyl.

The term “sulfo” as used herein is art recognized and refers to the group —SO₃H, or a salt form (such as a pharmaceutically acceptable salt) thereof.

Formulas indicating positively or negatively charged atoms or groups (such as N⁺ or SO₃⁻) mean salt forms of the respective formula (including “inner salts” in the case of zwitterions).

For purposes of the present invention, the term “salt” includes inorganic acid salts, such as hydrochloride, hydrobromide, sulfate, phosphate and the like; and organic acid salts, such as myristate, formate, acetate, trifluoroacetate, maleate, tartrate, bitartrate and the like; sulfonates, such as, methanesulfonate, benzenesultanate, p-toluenesulfonate and the like; and amino acid salts such as arginate, asparaginate, glutamate and the like. The term “salt” includes solvates, such as hydrates, of the respective salt.

In certain embodiments, the term “salt” as used herein means a diagnostically and/or pharmaceutically acceptable salt. In certain embodiments of the present invention wherein a compound is administered or is intended for administration to a subject, the term “salt” denotes a pharmaceutically acceptable salt, or a diagnostically and pharmaceutically acceptable salt.

The term “pharmaceutically acceptable salt”, as used herein, means a salt of a compound of the present invention which is safe and effective for topical or systemic use in mammals and that possesses the desired biological activity. The counter ion is suitable for the intended use, non-toxic, and it does not interfere with the desired biological action of the compound. Pharmaceutically acceptable salts in the context of the present invention include the salts reviewed in the lUPACHandbook of Pharmaceutically Acceptable Salts (Wermuth, C.G. and Stahl, P.H., Pharmaceutical Salts: Properties, Selection and Use - A Handbook, Verlag Helvetica Chimica Acta (2002)).

The term “diagnostically acceptable salt”, as used herein, refers to a salt of a compound of the present invention which is useful and effective for the desired diagnostic method. Its counter ion does not interfere with the reaction necessary for detection of the target protein, or with the method of detection.

In certain embodiments the compounds of the present invention are present as the trifluoroacetate salt, e.g., after HPLC-purification in an eluting solvent including trifluoroacetic acid (TFA).

In certain embodiments, “excipient” means a diagnostically and/or pharmaceutically acceptable excipient. In certain embodiments of the present invention wherein a composition is administered or is intended for administration to a subject, the term “excipient” denotes a pharmaceutically acceptable excipient, or a diagnostically and pharmaceutically acceptable excipient.

In formulas showing a curled line, the curled line represents or indicates the point of connection to the remainder of the molecule.

A compound of formula (I) (and optionally as further defined by formulas II and III) can contain one or more asymmetric centers and can thus give rise to enantiomers, diastereomers, and other stereoisomeric forms. Unless specifically otherwise indicated, the disclosure encompasses compounds with all such possible forms as well as their racemic and resolved forms or any mixture thereof. When a Compound of formula (I) contains an olefinic double bond or other center of geometric asymmetry, and unless specifically otherwise indicated, it is intended to include all “geometric isomers”, e.g., both E and Z geometric isomers. Unless specifically otherwise indicated, all “tautomers”, e.g., ketone-enol, amide-imidic acid, lactam-lactim, enamine-imine, amine-imine, and enamine-enimine tautomers, are intended to be encompassed by the disclosure as well.

As used herein, the terms “stereoisomer”, “stereoisomeric form”, and the like are general terms for all isomers of individual molecules that differ only in the orientation of their atoms in space. It includes enantiomers and isomers of compounds with more than one chiral center that are not mirror images of one another (“diastereomers”).

The term “chiral center” refers to a carbon atom to which four different groups are attached.

The term “enantiomer” or “enantiomeric” refers to a molecule that is nonsuperimposeable on its mirror image and hence optically active where the enantiomer rotates the plane of polarized light in one direction and its mirror image rotates the plane of polarized light in the opposite direction.

The term “racemic” refers to a mixture of equal parts of enantiomers which is optically inactive.

The term “resolution” refers to the separation or concentration or depletion of one of the two enantiomeric forms of a molecule. Optical isomers of a Compound of Formula (I) can be obtained by known techniques such as chiral chromatography or formation of diastereomeric salts from an optically active acid or base.

Optical purity can be stated in terms of enantiomeric excess (% ee), which is determined by the formula:

$$\% e e = \left[\frac{\text{major enantiomer} \left(\text{mol}\right) \text{- minor enantiomer} \left(\text{mol}\right)}{\text{major enantiomer} \left(\text{mol}\right) \text{+ minor enantiomer} \left(\text{mol}\right)}\right] \times 100 \% \qquad \text{[Math. 1]}$$

In one embodiment the invention relates to compounds having the absolute stereochemistry as indicated by formulas IA (and as optionally further defined by formulas IIA and IIIA).

The compounds of the present invention can be synthesized using standard synthetic chemical techniques, for example using the methods described in the Examples section below. Other useful synthetic techniques are described, for example, in March’s Advanced Organic Chemistry: Reactions, Mechanisms, and Structure, 7th Ed., (Wiley, 2013); Carey and Sundberg, Advanced Organic Chemistry 4thEd., Vols. A and B (Plenum 2000, 2001); Fiesers’ Reagents for Organic Synthesis, Volumes 1 -27 (Wiley, 2013); Rodd’s Chemistry of Carbon Compounds, Volumes 1-5 and Supplementals (Elsevier Science Publishers, 1989); Organic Reactions, Volumes 1-81 (Wiley, 2013); and Larock’s Comprehensive Organic Transformations (VCHPublishers Inc., 1989) (all of which are incorporated by reference in their entirety). The compounds are normally synthesized using starting materials that are generally available from commercial sources or are readily prepared using methods well known to those skilled in the art. See, e.g., Fiesers’ Reagents for Organic Synthesis, Volumes 1-27 (Wiley, 2013), or Beilsteins Handbuch der organischen Chemie, 4, Aufl. ed. Springer-Verlag, Berlin, including supplements.

### Compounds

In certain embodiments, the present invention is directed to a compound of formula I

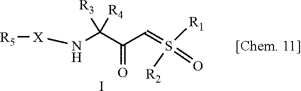

or a salt thereof, wherein
R₁ is selected from the group consisting of (C₁-C₈) alkyl, (C₁-C₈) hydroxyalkyl, (C₁-C₈) haloalkyl, (C₃-C₈) cycloalkyl, (C₂-C₈) alkenyl and (C₂-C₈) alkynyl;
R₂ is selected from the group consisting of (C₁-C₈) alkyl, (C₁-C₈) hydroxyalkyl, (C₁-C₈) haloalkyl, (C₃-C₈) cycloalkyl, (C₁-C₈) alkenyl and (C₂-C₈) alkynyl;
R₃ is the sidechain of an alpha amino acid;
R₄ is selected from the group consisting of hydrogen and (C₁-C₄) alkyl;
R₅ is selected from the group consisting of a detectable element, an amine protecting group, (C₁-C₈) alkyl, (C₁-C₈) hydroxyalkyl, (C₁-C₈) haloalkyl, (C₃-C₈ cycloalkyl, (C₂-C₈) alkenyl, (C₂-C₈) alkynyl, (C₁-C₈) alkylcarbonyl, (C₁-C₈) hydroxyalkylcarbonyl, (C₁-C₈) haloalkylcarbonyl, (C₃-C₈) cycloalkylcarbonyl, (C₁-C₈) alkyloxycarbonyl, benzyloxycarbonyl, and hydrogen;
X is
   (i) a bond; or
   (ii) a biradical moiety of formula II or III which is connected to the R₅ substituent via the amino group

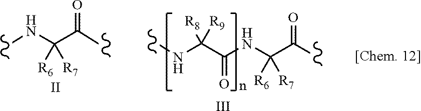

wherein
R₆ is the sidechain of an alpha amino acid;
R₇ is selected from the group consisting of hydrogen and (C₁-C₄) alkyl;
Rs is the sidechain of an alpha amino acid;
R₉ is selected from the group consisting of hydrogen and (C₁-C₄) alkyl; and n is 1, 2, 3, or 4.

In certain embodiments, the compound of formula I is characterized in that R₃ is the sidechain of a natural alpha amino acid, or a structural isomer, homologue and/or structural analogue of said sidechain, wherein said sidechain or structural isomer, homologue and/or structural analogue thereof is optionally substituted by an amine protecting group or a detectable element and optionally further substituted by one or more, same or different substituents R₃ₓ, wherein R₃ₓ is selected from the group consisting of hydroxy, halogen, (C₁-C₄) alkyl, (C₁-C₄) hydroxyalkyl, (C₁-C₄) haloalkyl, (C₁-C₄) alkoxy, and (C₁-C₄) haloalkoxy.

In certain other embodiments, R₃ is the sidechain of a proteinogenic alpha amino acid, or a structural isomer, homologue and/or structural analogue of said sidechain, wherein said sidechain or structural isomer, homologue and/or structural analogue thereof is optionally substituted by an amine protecting group or a detectable element and optionally further substituted by one or more, same or different substituents R₃ₓ, wherein R₃ₓ is selected from the group consisting of hydroxy, halogen, (C₁-C₄) alkyl, (C₁-C₄) hydroxyalkyl, (C₁-C₄) haloalkyl, (C₁-C₄) alkoxy, and (C₁-C₄) haloalkoxy.

In certain embodiments, R₃ is the sidechain of a proteinogenic alpha amino acid except lysine, or a structural isomer or homologue of said sidechain, wherein said sidechain or structural isomer or homologue thereof is optionally substituted by an amine protecting group or a detectable element and optionally further substituted by one or more, same or different substituents R₃ₓ, wherein R₃ₓ is selected from the group consisting of hydroxy, halogen, (C₁-C₄) alkyl, (C₁-C₄) hydroxyalkyl, (C₁-C₄) haloalkyl, (C₁-C₄) alkoxy, and (C₁-C₄) haloalkoxy.

In certain embodiments, R₃ is the sidechain of an alpha amino acid selected from the group consisting of glycine, alanine, alpha-aminobutyric acid, valine, norvaline, leucine, isoleucine, norleucine, homonorleucine, methionine, ethionine, phenylalanine, tyrosine, levodopa, tryptophan, cysteine, homocysteine, selenocysteine, selenohomocysteine, selenomethionine, selenoethionine, lysine, histidine, arginine, ornithine, aspartic acid, glutamic acid, serine, homoserine, O-methyl-homoserine, O-ethyl-homoserine, threonine, asparagine, and glutamine, or a structural isomer, homologue and/or structural analogue of said sidechain, wherein said sidechain or structural isomer, homologue and/or structural analogue thereof is optionally substituted by an amine protecting group or a detectable element and optionally further substituted by one or more, same or different substituents R₃ₓ, wherein R₃ₓ is selected from the group consisting of hydroxy, halogen, (C₁-C₄) alkyl, (C₁-C₄) hydroxyalkyl, (C₁-C₄) haloalkyl, (C₁-C₄) alkoxy, and (C₁-C₄) haloalkoxy.

In certain embodiments, R₃ is the sidechain of an alpha amino acid selected from the group consisting of glycine, alanine, alpha-aminobutyric acid, valine, norvaline, leucine, isoleucine, norleucine, homonorleucine, methionine, ethionine, phenylalanine, tyrosine, levodopa, tryptophan, cysteine, homocysteine, selenocysteine, selenohomocysteine, selenomethionine, selenoethionine, histidine, arginine, ornithine, aspartic acid, glutamic acid, serine, homoserine, O-methyl-homoserine, O-ethyl-homoserine, threonine, asparagine, and glutamine, or a structural isomer or homologue of said sidechain, wherein said sidechain or structural isomer or homologue thereof is optionally substituted by an amine protecting group or a detectable element and optionally further substituted by one or more, same or different substituents R₃ₓ, wherein R₃ₓ is selected from the group consisting of hydroxy, halogen, (C₁-C₄) alkyl, (C₁-C₄) hydroxyalkyl, (C₁-C₄) haloalkyl, (C₁-C₄) alkoxy, and (C₁-C₄) haloalkoxy. In certain such embodiments, the alpha amino acid is selected from the group consisting of glycine, alanine, alpha-aminobutyric acid, valine, norvaline, leucine, isoleucine, norleucine, homonorleucine, methionine, ethionine, phenylalanine, tyrosine, levodopa, tryptophan, cysteine, homocysteine, selenocysteine, selenohomocysteine, selenomethionine, and selenoethionine. In certain such embodiments, the alpha amino acid is selected from the group consisting of alanine, alpha-aminobutyric acid, valine, norvaline, leucine, isoleucine, norleucine, homonorleucine, phenylalanine, and tryptophan. In certain such embodiments, the alpha amino acid is selected from the group consisting of valine, norvaline, leucine, isoleucine, norleucine, and homonorleucine. In certain such embodiments, the alpha amino acid is selected from the group consisting of valine, leucine, isoleucine, and norleucine. In certain such embodiments, the alpha amino acid is leucine or norleucine. In certain such embodiments, the alpha amino acid is norleucine.

In certain embodiments, the compound of formula I is characterized in that R₃ is selected from the group consisting of hydrogen, (C₁-C₈) alkyl, (C₁-C₈) hydroxyalkyl, (C₁-C₈) haloalkyl, (C₃-C₈) cycloalkyl, (C₁-C₈) alkenyl, (C₂-C₈) alkynyl, (C₆-C₁₀) arylmethyl, (C₃-C₉) heteroarylmethyl, and -CH₂CH₂CH2CH2N(R₃ₐ)(R3b); wherein R₃ₐ is selected from the group consisting of a detectable element, an amine protecting group, hydrogen, and (C₁-C₈) alkyl; and R_{3b} is selected from the group consisting of hydrogen and (C₁-C₄) alkyl. In certain such embodiments, R₃ is selected from the group consisting of (C₁-C₈) alkyl, (C₂-C₈) alkenyl, (C₂-Cs) alkynyl, (C₆-C₁₀) arylmethyl, (C₃-C₉) heteroarylmethyl, and -CH₂CH₂CH₂CH₂N(RJₐ)(R3_{b}). In certain such embodiments, R₃ is selected from the group consisting of (C₁-C₈) alkyl, (C₆-C₁₀) arylmethyl, (C₃-C₉) heteroarylmethyl, and -CH₂CH₂CH₂CH₂N(R₃ₐ)(R_{3b}). In certain such embodiments, R₃ is selected from the group consisting of (C₁-C₈) alkyl, benzyl, (1H-indol-3-yl) methyl, and -CH₂CH2CH₂CH₂N(R₃ₐ)(R_{3b}) In certain such embodiments, R₃ is selected from the group consisting of (C₁-C₆) alkyl, benzyl, (1H-indol-3-yl) methyl, and -CH₂CH₂CH₂CH₂N(R₃ₐ)(R_{3b}). In certain such embodiments, R₃ is selected from the group consisting of ethyl, n-propyl, iso-propyl, n-butyl, sec-butyl, iso-butyl, n-pentyl, benzyl, (1H-indol-3-yl) methyl, and -CH₂CH₂CH₂CH2N(R₃ₐ)(R_{3b}). In certain such embodiments, R_{3b} is hydrogen.

In certain such embodiments, R₃ₐ is not an amine protecting group, hydrogen, or (C₁-C₈) alkyl, if X is a bond. In other such embodiments, R₃ is not -CH₂CH₂CH₂CH₂N(R₃ₐ)(R_{3b}), if X is a bond.

In other such embodiments, R₃ₐ is a detectable element (irrespective of the nature of X). In other such embodiments, R₃ₐ is hydrogen. In other such embodiments, R₃ₐ is an amine protecting group. In other such embodiments, R₃ₐ is (C₁-C₈) alkyl.

In certain embodiments, R_{3b} is hydrogen.

In certain embodiments, the compound of formula I is characterized in that R₃ is selected from the group consisting of hydrogen, (C₁-C₈) alkyl, (C₁-C₈) hydroxyalkyl, (C₁-C₈) haloalkyl, (C₃-C₈) cycloalkyl, (C₂-C₈) alkenyl, (C₂-C₈) alkynyl, (C₆-C₁₀) arylmethyl, and (C₃-C₉) heteroarylmethyl. In certain such embodiments, R₃ is selected from the group consisting of (C₁-C₈) alkyl, (C₂-C₈) alkenyl, (C₂-C₈) alkynyl, (C₆-C₁₀) arylmethyl, and (C₃-C₉) heteroarylmethyl. In certain such embodiments, R₃ is selected from the group consisting of (C₁-C₈) alkyl, (C₆-C₁₀) arylmethyl, and (C₃-C₉) heteroarylmethyl. In certain such embodiments, R₃ is selected from the group consisting of (C₁-C₈) alkyl, benzyl, and (1H-indol-3-yl) methyl. In certain such embodiments, R₃ is selected from the group consisting of (C₁-C₈) alkyl, benzyl, and (1H-indol-3-yl) methyl. In certain such embodiments, R₃ is selected from the group consisting of ethyl, n-propyl, iso-propyl, n-butyl, sec-butyl, iso-butyl, n-pentyl, benzyl, and (1H-indol-3-yl) methyl. In certain such embodiments, R₃ is selected from the group consisting of n-butyl, sec-butyl, and iso-butyl. In certain such embodiments, R₃ is n-butyl.

In certain embodiments, the compound of formula I is characterized in that R₃ is -CH₂CH₂CH₂CH₂N(R₃ₐ)(R_{3b}); wherein
R₃ₐ is selected from the group consisting of a detectable element, an amine protecting group, hydrogen, and (C₃-C₈) alkyl; and
R_{3b} is selected from the group consisting of hydrogen and (C₁-C₄) alkyl. In certain such embodiments, R₃ₐ is not an amine protecting group, hydrogen, or (C₁-C₈) alkyl, if X is a bond. In other such embodiments, R₃ₐ is hydrogen (irrespective of the nature of X). In other such embodiments, R₃ₐ is an amine protecting group. In other such embodiments, R₃ₐ is (C₁-C₈) alkyl. In certain embodiments, R_{3b} is hydrogen.

In certain embodiments, R₁ is (C₁-C₈) alkyl. In certain such embodiments, R₁ is methyl.

In certain embodiments, R₂ is (C₁-C₈) alkyl. In certain such embodiments, R₂ is methyl.

In certain embodiments, R₄ is hydrogen.

In certain embodiments, R₅ is selected from the group consisting of a detectable element, an amine protecting group, and hydrogen.

In certain embodiments, R₆, is
(i) the sidechain of a natural alpha amino acid, or
(ii) the sidechain of a proteinogenic alpha amino acid, or
(iii) the sidechain of an alpha amino acid selected from the group consisting of glycine, alanine, alpha-aminobutyric acid, valine, norvaline, leucine, isoleucine, norleucine, homonorleucine, methionine, ethionine, phenylalanine, tyrosine, levodopa, tryptophan, cysteine, homocysteine, selenocysteine, selenohomocysteine, selenomethionine, selenoethionine, lysine, histidine, arginine, ornithine, aspartic acid, glutamic acid, serine, homoserine, O-methyl-homoserine, O-ethyl-homoserine, threonine, asparagine, and glutamine, or
(iv) the sidechain of phenylalanine or a structural isomer, homologue and/or structural analogue of said sidechain,
wherein said sidechain or structural isomer, homologue and/or structural analogue thereof is optionally substituted by an amine protecting group or a detectable element and optionally further substituted by one or more, same or different substituents R₆ₓ, wherein

R₆ₓ is selected from the group consisting of hydroxy, halogen, (C₁-C₄) alkyl, (C₁-C₄) hydroxyalkyl, (C₁-C₄) haloalkyl, (C₁-C₄) alkoxy, and (C₁-C₄) haloalkoxy.

In certain embodiments, R₆ is the sidechain of phenylalanine, or a structural isomer, homologue and/or structural analogue of said sidechain. In certain such embodiments, R₆ is the sidechain of phenylalanine.

In certain embodiments, R₈ is
(i) the sidechain of a natural alpha amino acid, or
(ii) the sidechain of a proteinogenic alpha amino acid, or
(iii) the sidechain of an alpha amino acid selected from the group consisting of glycine, alanine, alpha-aminobutyric acid, valine, norvaline, leucine, isoleucine, norleucine, homonorleucine, methionine, ethionine, phenylalanine, tyrosine, levodopa, tryptophan, cysteine, homocysteine, selenocysteine, selenohomocysteine, selenomethionine, selenoethionine, lysine, histidine, arginine, ornithine, aspartic acid, glutamic acid, serine, homoserine, O-methyl-homoserine, O-ethyl-homoserine, threonine, asparagine, and glutamine, or
(iv) the sidechain of phenylalanine or a structural isomer, homologue and/or structural analogue of said sidechain,
wherein said sidechain or structural isomer, homologue and/or structural analogue thereof is optionally substituted by an amine protecting group or a detectable element and optionally further substituted by one or more, same or different substituents R₈ₓ, wherein

R₈ₓ is selected from the group consisting of hydroxy, halogen, (C₁-C₄) alkyl, (C₁-C₄) hydroxyalkyl, (C₁-C₄) haloalkyl, (C₁-C₄) alkoxy, and (C₁-C₄) haloalkoxy.

In certain embodiments, R₇ is hydrogen.

In certain embodiments, R₉ is hydrogen.

In certain embodiments, where a substituent is an amine protecting group, the amine protecting group can be selected from the group consisting of benzyloxycarbonyl (Cbz), 9-fluorenylmethyloxycarbonyl (Fmoc), tert-butyloxycarbonyl (Boc), allyloxycarbonyl (Alloc), p-toluenesulfonyl (Tos), 2,2,5,7,8-pentamethylchroman-6-sulfonyl (Pmc), 2,2,4,6,7-pentamethyl-2,3-dihydrobenzofuran-5-sulfonyl (Pbf), mesityl-2-sulfonyl (Mts), 4-methoxy-2,3,6-trimethylphenylsulfonyl (Mtr), acetamido, and phthalimido. In certain such embodiments the amine protecting group is benzyloxycarbonyl.

In certain of the above embodiments, the compound of formula I exhibits the absolute stereochemistry according to formula IA:

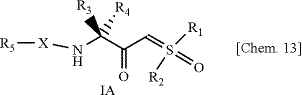

In certain such embodiments, in the compound of formula I or IA, X is (i) a bond, or (ii) a biradical moiety of formula II or III exhibiting the absolute stereochemistry according to formula IIA and IIIA, respectively:

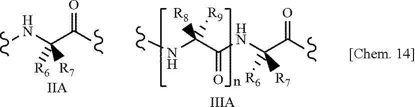

In certain embodiments, X is a bond.

In certain other embodiments, X is a biradical moiety of formula II. In certain such embodiments, X is a biradical moiety of formula IIA.

In certain other embodiments, X is a biradical moiety of formula III. In certain such embodiments, X is a biradical moiety of formula IIIA. In certain such embodiments, n is 1.

In certain embodiments, the detectable element is selected from the group consisting of a fluorescent label, a biotin label, a radiolabel, a chelator (e.g., for a radiolabel), and a bioorthogonal ligation handle.

The detectable element, such as the fluorescent label, biotin label, radiolabel, chelator or bioorthogonal ligation handle, can include a linker for incorporation into the compounds of the present invention (i.e., for attachment of the detectable element or label to the remainder of the molecule). Suitable linkers are known to those of skill in the art. Examples of linkers which can be used in the compounds of the present invention are described in WO 2012/118715 A2 (see page 18, lines 9-18), the contents of which are hereby included into the present disclosure. The linker can also include a polyethylene glycol (PEG) moiety, such as PEG-4, PEG-6 or PEG-8 for attachment to the remainder of the molecule.

A definition of the term “radiolabel” and examples of radiolabels which can be used in the compounds of the present invention are described in WO 2009/124265 A1 (see page 11, line 25 to page 13, line 3), the contents of which are hereby included into the present disclosure.

A definition of the term “chelator” and examples of chelators which can be used in the compounds of the present invention are described in WO2009/124265 A1 (see page 10, line 26 to page 11, line 14), the contents of which are hereby included into the present disclosure.

A definition of the term “bioorthogonal ligation handle” and examples of bioorthogonal ligation handles which can be used in the compounds of the present invention and respective “click” reactions are described, e.g., in Martell et al., Applications of Copper-Catalyzed Click Chemistry in Activity-Based Protein Profiling, Molecules 2014, 19, 1378-1393, which is incorporated herein by reference. Adaptation of these methods to generate or modify compounds of the instant claims is within the skill in the art.

Bioorthogonal or click reactions for attachment of the secondary label include
A. the traceless Staudinger Ligation coupling azides with triarylphosphines to generate an amide linkage,
B. the tetrazine cycloaddition utilizing a 1,2,4,5-tetrazine and a strained diene (trans-cyclooctene),
C. the copper (I)-catalyzed azide-alkyne cycloaddition (CuAAC) reaction between an azide and a terminal alkyne to generate a 1,4-disubstituted 1,2,3-triazole, and
D. the copper-free variant of the azide-alkyne cycloaddition utilizing a strained alkyne to accelerate the reaction.

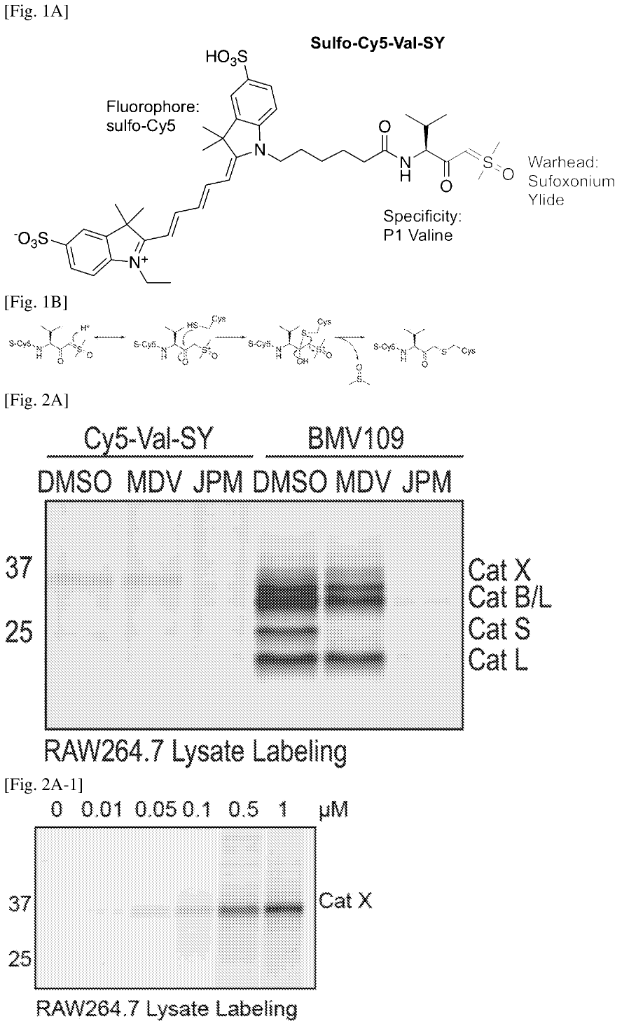

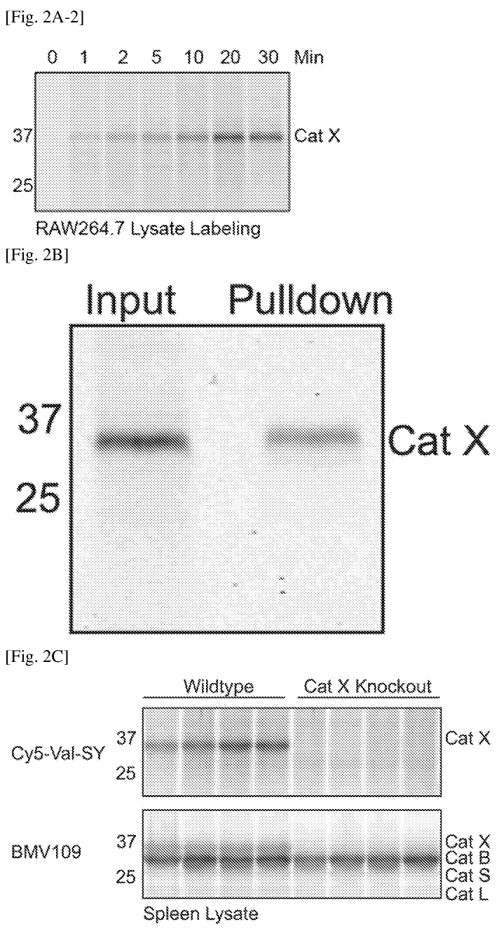

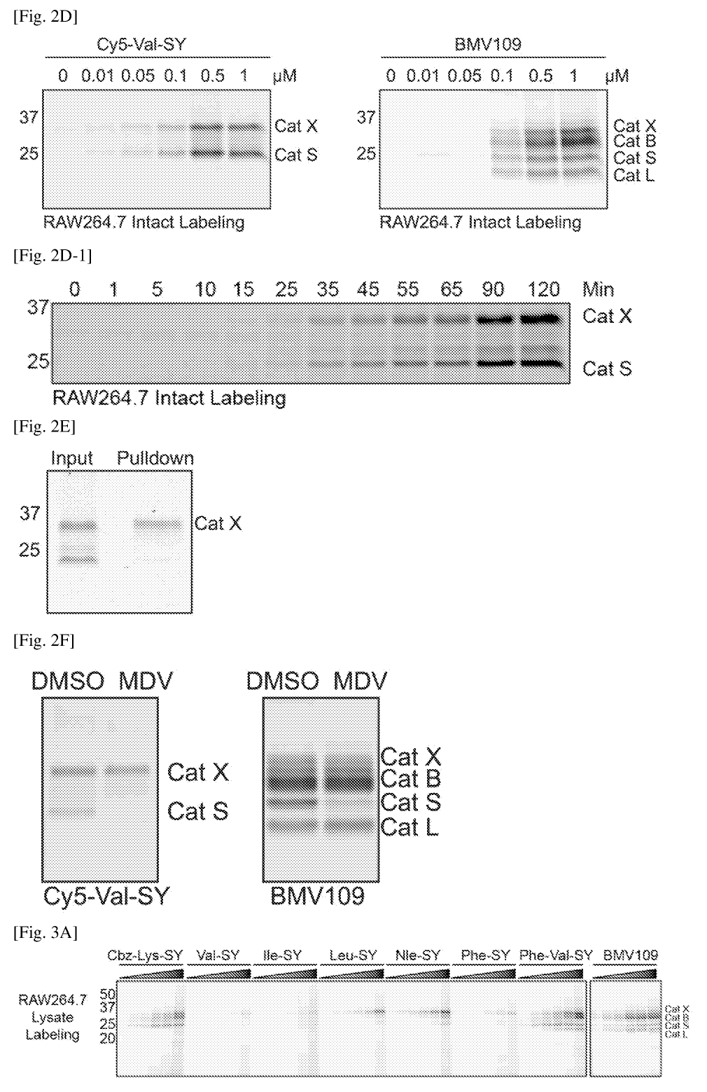

In this regard, reference is particularly made to FIG. 1B and FIG. 2 of Martell et al., Molecules 2014, 19, 1378-1393, the contents of which are hereby included into the present disclosure.

Thus, in certain embodiments, the bioorthogonal ligation handle comprises a functional group selected from the group consisting of an azide, a 1,2,4,5-tetrazine, and an alkyne (such as a terminal alkyne). These functional groups allow the attachment of a secondary label using one of the above bioorthogonal reactions (A) to (D).

In certain embodiments, the detectable element is a fluorescent label. As is known by those of skill in the art, fluorescent labels emit electromagnetic radiation, preferably visible light, when stimulated by the absorption of incident electromagnetic radiation. A wide variety of fluorescent labels, including labels having reactive moieties useful for coupling the label to reactive groups such as, for example amino groups, thiol groups and the like, are commercially available. See, e.g., The Molecular Probes&reg; Handbook- A guide to Fluorescent Probes and Labeling technologies, which is hereby incorporated by reference in its entirety.

Examples of fluorescent labels which can be used in the compounds of the present invention are described in WO 2018/119476 A1 (see paragraphs [0084] to [0095]) and in WO 2012/118715 A2 (see page 15, line 18 to page 17, line 12, and page 18, line 19 to page 21, line 1), the contents of which are hereby included in the present disclosure. Such fluorescent labels can include a linker for incorporation into the compounds of the present invention, e.g., as described in WO 2012/118715 A2 (see page 18, lines 9-18), the contents of which are hereby included into the present disclosure.

In certain embodiments, the detectable element is a fluorescent label. In certain such embodiments, the fluorescent label is selected from the group consisting of a fluorescein, an Oregon green (a fluorinated derivative of fluorescein), a bora-diaza-indecene dye, a rhodamine dye (such as tetramethylrhodamine and carboxy tetramethyl rhodamine), a benzopyrillium dye, a coumarin dye, a cyanine label or a benzoindole label (such as indocyanine green).

Commercially available examples of such dyes include the BODIPY&reg; dyes (bora-diaza-indecene dyes), dyes of the Alexa Fluor&reg_{:} series (sulfonated rhodamines), dyes of the DyLight&reg; series (having e.g. a sulfonated or unsulfonated coumarin, rhodamine, benzopyrilium, or cyanine as base structure), dyes of the IRDye&reg; series, and cyanine (Cy) dyes (e.g. Cy2, Cy3, Cy3.5, Cy5, Cy5.5, Cy7, Cy7.5, sCy3, sCy5, and sCy7). Such cyanine labels can be purchased, e.g., from the companies Abcam, Tocris, GoldBio, ThermoFisher, Kerafast, Lumiprobe, AAT Bioquest or W&J Pharmachem.

In certain embodiments the fluorescent label is a cyanine label. In certain such embodiments the fluorescent label is a cyanine label selected from the group consisting of Cy2, Cy3, Cy3.5, Cy5, Cy5.5, Cy7, Cy7.5, sCy3, sCy5, and sCy7. In certain such embodiments the fluorescent label is Cy5 or sCy5. In certain embodiments the fluorescent label is sCy5.

In certain embodiments the fluorescent label is a cyanine label having a formula selected from the following group of formulas:

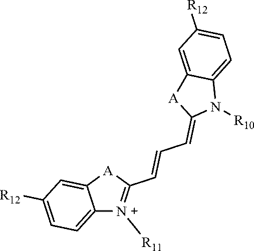

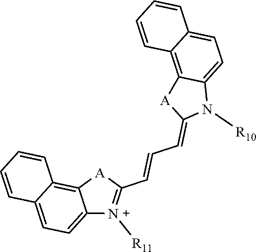

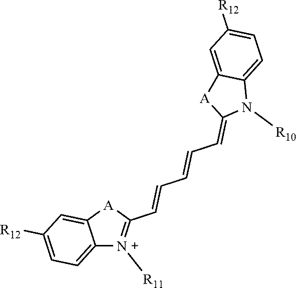

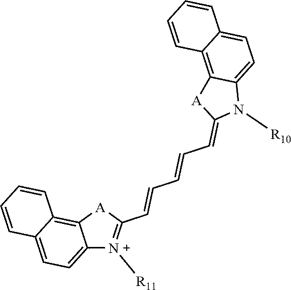

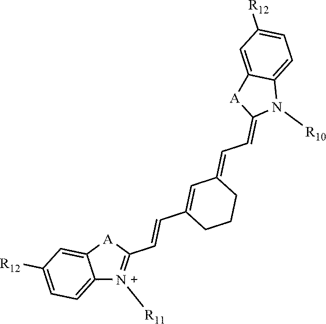

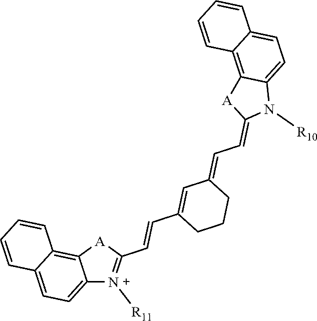

wherein in each of the above formulas,
A is selected from the group consisting of CH₂, C(CH₃)₂, C(C₂H₅)2, NH, N(CH₃), N(C₂H₅),
O, S, and Se;
R₁₀ is selected from the group consisting of $-(CH₂)ₚ-C(=O)-& and $-(CH₂)_{q}-C(=O)-NH-[CH₂CH₂O]ᵣ-CH₂CH₂-C(=O)-&;
wherein
p is 2, 3, 4, 5, 6, 7, or 8;
q is 2, 3, 4, 5, 6, 7, or 8;
r is 2, 3, 4, 5, 6, 7, or 8;
$ represents the point of connection to the nitrogen atom of the cyanine moiety;
and & represents the point of connection to the remainder of the molecule;
R₁₁ is selected from the group consisting of (C₁-C₈)alkyl, and (C₆-C₁₀)aryl; and
R₁₂ is H or a sulfo group. In certain such embodiments, p is 5, q is 5 and r is 4.

In certain embodiments wherein the fluorescent label is a cyanine label having one of the above formulas,
A is selected from the group consisting of CH₂, C(CH₃)₂, and C(C₂H₅)₂;
R₁₀ is selected from the group consisting of $-(CH₂)ₚ-C(=O)-& and $-(CH₂)_{q}-C(=O)-NH-[CH₂CH₂O]ᵣ-CH₂CH₂-C(=O)-&;
wherein
p is 2, 3, 4, 5, or 6;
q is 2, 3, 4, 5, or 6;
r is 2, 3, 4, 5, or 6;
$ represents the point of connection to the nitrogen atom of the cyanine moiety;
and & represents the point of connection to the remainder of the molecule;
R₁₁ is (C₁-C₈)alkyl_{:} and
R₁₂ is Hor a sulfo group. In certain such embodiments, p is 5, q is 5 and r is 4.

In certain embodiments wherein the fluorescent label is a cyanine label having one of the above formulas,
A is C(CH₃)₂ or C(C₂H₅)₂;
R₁₀ is selected from the group consisting of $-(CH₂)ₚ-C(=O)-& and $-(CH₂)_{q}-C(=O)-NH-[CH₂CH₂O]ᵣCH₂CH₂-C(=O)-&_{;}
wherein
p is 2, 3, 4, 5, or 6;
q is 2, 3, 4, 5, or 6;
r is 2, 3, 4, 5, or 6;
$ represents the point of connection to the nitrogen atom of the cyanine moiety;
and & represents the point of connection to the remainder of the molecule;
R₁₁ is methyl, ethyl or propyl; and
R₁₂ is H or a sulfo group. In certain such embodiments, p is 5, q is 5 and r is 4.

In certain embodiments wherein the fluorescent label is a cyanine label having one of the above formulas,
A is C(CH₃)₂;
R₁₀ is selected from the group consisting of $-(CH₂)ₚ₋C(=O)-& and $-(CH₂)_{q}-C(=O)-NH-[CH₂CH₂O]ᵣ-CH₂CH₂-C(=O)-&;
wherein
p is 4, 5, or 6;
q is 4, 5, or 6;
r is 3, 4, 5, or 6;
$ represents the point of connection to the nitrogen atom of the cyanine moiety;
and & represents the point of connection to the remainder of the molecule;
R₁₁ is methyl or ethyl; and
R₁₂ is H or a sulfo group. In certain such embodiments, p is 5, q is 5 and r is 4.

In certain embodiments wherein the fluorescent label is a cyanine label having one of the above formulas,
A is C(CH₃) ₂;
R₁₀ is $-(CH₂)ₚ-C(=O)-&; wherein
p is 4, 5, or 6; and
$ represents the point of connection to the nitrogen atom of the cyanine moiety;
and & represents the point of connection to the remainder of the molecule;
R₁₁ is methyl or ethyl; and
R₁₂ is a sulfo group. In certain such embodiments, p is 5.

In certain embodiments wherein the fluorescent label is a cyanine label having one of the above formulas,
A is C(CH₃)₂;
R₁₀ is $-(CH₂)_{q}-C(=O)-NH-[CH₂CH₂O]ᵣ-CH₂CH₂-C(=O)-&;
wherein
q is 4, 5, or 6;
r is 3, 4, 5, or 6;
$ represents the point of connection to the nitrogen atom of the cyanine moiety;
and & represents the point of connection to the remainder of the molecule;
R₁₁ is methyl or ethyl; and
R₁₂ is H. In certain such embodiments, q is 5 and r is 4.

In certain embodiments, wherein the fluorescent label is a cyanine label having one of the above formulas, R₅ is a detectable element, and & represents the point of connection to X.

In certain embodiments, the fluorescent label is a cyanine label having a formula selected from the following group of formulas:

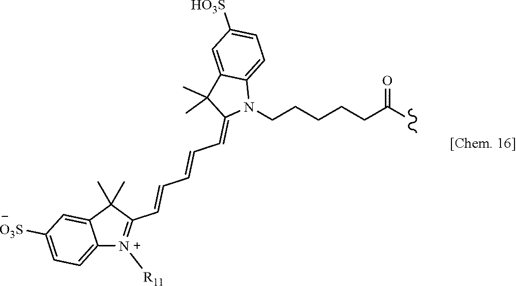

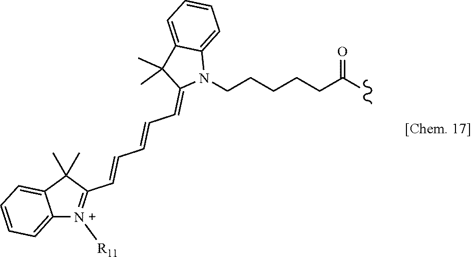

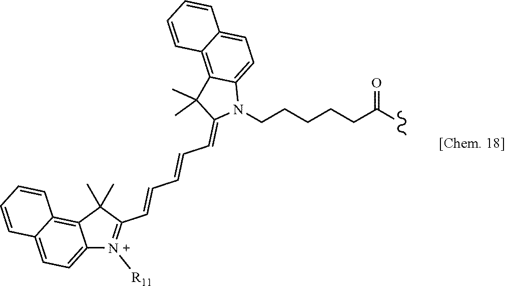

wherein in each of the above formulas, the curled line represents the point of connection to the remainder of the molecule; and R₁₁ is selected from the group consisting of (C₁-C₈)alkyl, and (C₆-C₁₀)aryl. In certain such embodiments, R₁₁ is (C₁-C₈)alkyl. In certain such embodiments, R₁₁ is methyl or ethyl.

In certain embodiments, the fluorescent label is a cyanine label having the formula

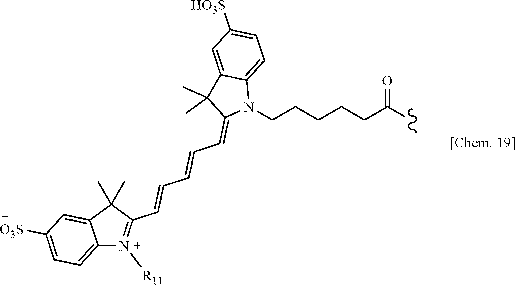

wherein the curled line represents the point of connection to the remainder of the molecule; and R₁₁ is methyl or ethyl.

In certain embodiments, wherein the fluorescent label is a cyanine label having one of the above formulas, R₅ is a detectable element, and the curled line represents the point of connection to X.

In certain embodiments, the compound of formula I (or IA) comprises at least one detectable element, such as one, two or three detectable elements. In certain such embodiments the compound of formula I (or IA) as defined above comprises one detectable element.

In certain embodiments, R₅ is a detectable element.

In certain embodiments, R₃ bears a detectable element. In certain such embodiments, R₃ is the sidechain of lysine, or a structural isomer, homologue and/or structural analogue of said sidechain, wherein said sidechain or structural isomer, homologue and/or structural analogue thereof is substituted by a detectable element and optionally further substituted by one or more, same or different substituents R₃ₓ, wherein R₃ₓ is selected from the group consisting of hydroxy, halogen, (C₁-C₄) alkyl, (C₁-C₄) hydroxyalkyl, (C₁-C₄) haloalkyl, (C₁-C₄) alkoxy, and (C₁-C₄) haloalkoxy. In certain such embodiments, R₃ is -CH₂CH₂CH₂CH₂N(R₃ₐ)(R_{3b}); wherein R₃ₐ is a detectable element; and R_{3b} is selected from the group consisting of hydrogen and (C₁-C₄) alkyl.

In certain embodiments wherein R₃ bears a detectable element, R₅ is selected from the group consisting of an amine protecting group, (C₁-C₈) alkyl, (C₁-C₈) hydroxyalkyl, (C₁-C₈) haloalkyl, (C₃-C₈) cycloalkyl, (C₂-C₈) alkenyl, (C₂-C₈) alkynyl, (C₁-C₈) alkylcarbonyl, (C₁-C₈) hydroxyalkylcarbonyl, (C₁-C₈) haloalkylcarbonyl, (C₃-C₈) cycloalkylcarbonyl, (C₁-C₈) alkyloxycarbonyl, benzyloxycarbonyl, and hydrogen. In certain such embodiments, R₅ is an amine protecting group.

In certain embodiments, X is a biradical moiety of formula II or IIA, and R₆ bears a detectable element. In certain such embodiments, R₆ is the sidechain of lysine, or a structural isomer, homologue and/or structural analogue of said sidechain, wherein said sidechain or structural isomer, homologue and/or structural analogue thereof is substituted by a detectable element and optionally further substituted by one or more, same or different substituents R₆ₓ, wherein R₆ₓ is selected from the group consisting of hydroxy, halogen, (C₁-C₄) alkyl, (C₁-C₄) hydroxyalkyl, (C₁-C₄) haloalkyl, (C₁-C₄) alkoxy, and (C₁-C₄) haloalkoxy.

In certain embodiments, X is a biradical moiety of formula III or IIIA, and R₆ bears a detectable element. In certain such embodiments, R₆ is the sidechain of lysine, or a structural isomer, homologue and/or structural analogue of said sidechain, wherein said sidechain or structural isomer, homologue and/or structural analogue thereof is substituted by a detectable element and optionally further substituted by one or more, same or different substituents R₆ₓ, wherein R₆ₓ is selected from the group consisting of hydroxy, halogen, (C₁-C₄) alkyl, (C₁-C₄) hydroxyalkyl, (C₁-C₄) haloalkyl, (C₁-C₄) alkoxy, and (C₁-C₄) haloalkoxy.

In certain embodiments, X is a biradical moiety of formula III or IIIA, and R₈ bears a detectable element. In certain such embodiments, and R₈ is the sidechain of lysine, or a structural isomer, homologue and/or structural analogue of said sidechain, wherein said sidechain or structural isomer, homologue and/or structural analogue thereof is substituted by a detectable element and optionally further substituted by one or more, same or different substituents R₈ₓ, wherein R₈ₓ is selected from the group consisting of hydroxy, halogen, (C₁-C₄) alkyl, (C₁-C₄) hydroxyalkyl, (C₁-C₄) haloalkyl, (C₁-C₄) alkoxy, and (C₁-C₄) haloalkoxy.

In certain embodiments, the compound of formula I is a compound having one of the following formulas (with absolute stereochemistry as indicated):

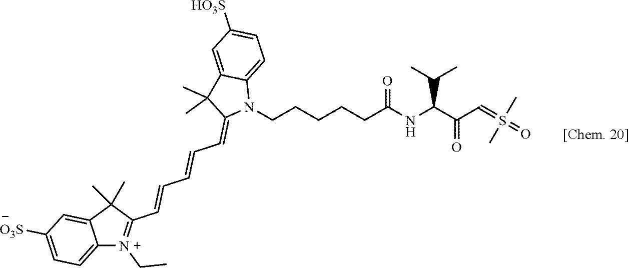

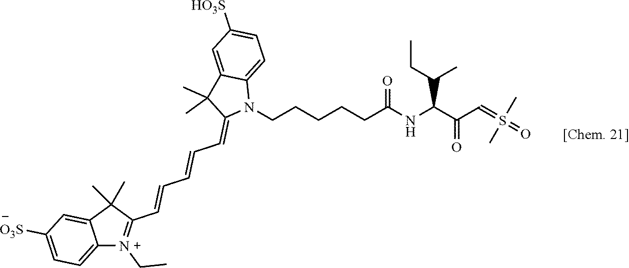

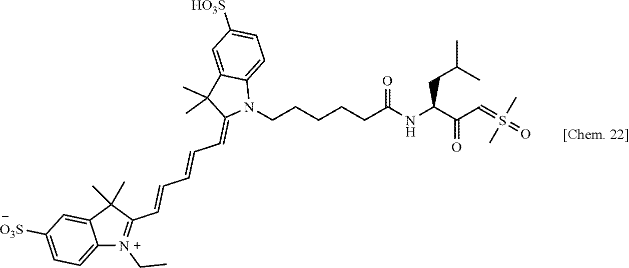

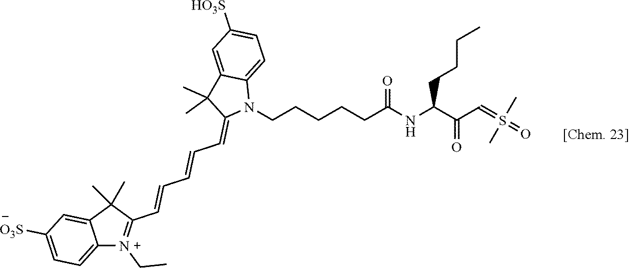

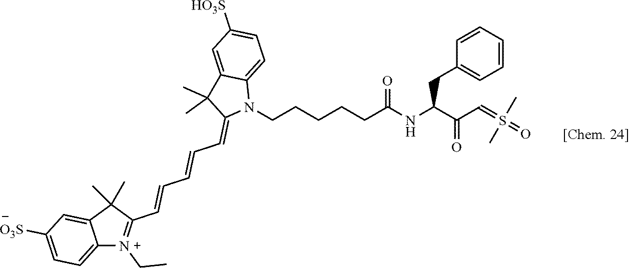

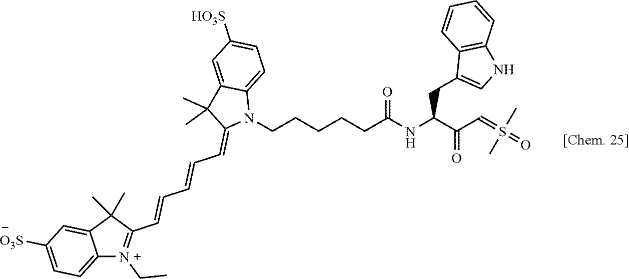

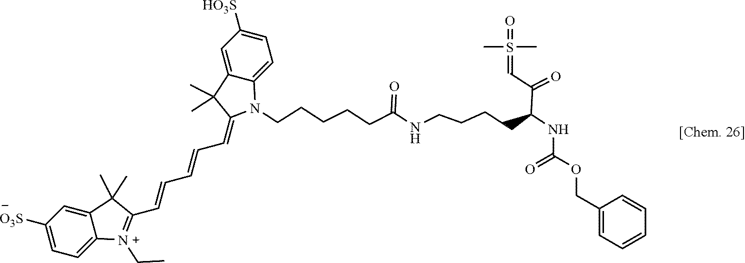

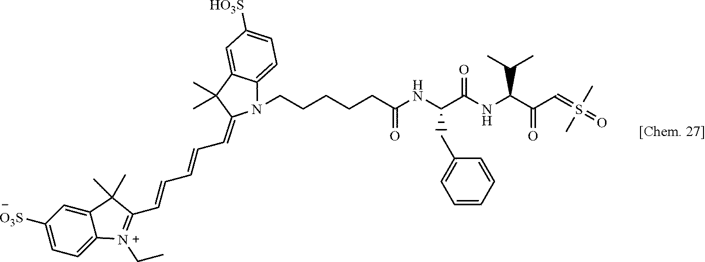

or a salt thereof.

In certain preferred embodiments the compound of formula I is a compound having the following formula (with absolute stereochemistry as indicated):

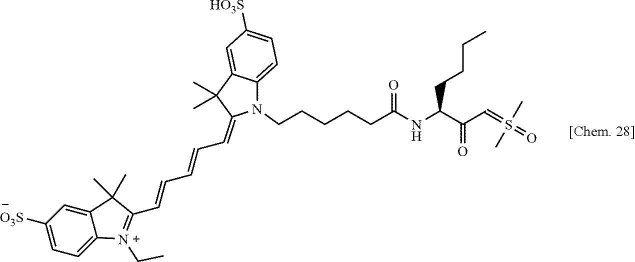

or a salt thereof.

### Compositions

In certain embodiments the present invention relates to a composition comprising a compound of formula I (or IA) as described above, or a salt thereof, and an excipient (e.g., a pharmaceutically and/or diagnostically acceptable excipient).

Pharmaceutically acceptable excipients that can be used in the compositions of the present invention are known to the skilled person. Examples of such pharmaceutically acceptable excipients include, e.g. those described in paragraphs [0114] to [0118] of WO 2018/119476, the contents of which are hereby introduced into the present disclosure.

In certain embodiments the composition is an aqueous solution comprising e.g. water, physiologically buffered saline or a buffer solution as pharmaceutically acceptable excipient. Such compositions can be used, e.g., for intravenous injection.

### Methods of Detecting Cysteine Protease Activity

In the methods of detecting cysteine protease activity according to the present invention, only proteolytically active forms of the respective cysteine protease(s) are detected.

The detectable signal is measured after a reaction between the activity-based probe compound and the cysteine protease has taken place, which has resulted in the formation of a covalent bond. The measured detectable signal is emitted by the labelled enzyme, i.e. by the detectable element of the activity-based probe compound covalently attached to the cysteine protease. In certain embodiments the detectable signal is measured after subjecting the labelled enzyme to a secondary labeling step.

The concept of detecting enzyme activity using activity-based probes and respective methods of detection and underlying experimental protocols are known to the skilled person (see, e.g., Edgington and Bogyo, 2013; Edgington-Mitchell, L.E., and Bogyo, M. (2016). Detection of Active Caspases During Apoptosis Using Fluorescent Activity-Based Probes. Methods Mol Biol. 1419, 27-39; and Edgington-Mitchell, L.E., Bogyo, M., and Verdoes, M. (2017). Live Cell Imaging and Profiling of Cysteine Cathepsin Activity Using a Quenched Activity-Based Probe. Methods Mol Biol. 1491, 145-159; the contents of which are hereby incorporated by reference in their entirety). The skilled person knows how to suitably adapt these methods/protocols for use in the methods of the present invention.

### In Vitro Methods of Detection

In certain embodiments, the present invention is directed to a method of detecting cysteine protease activity comprising
(1) contacting the cysteine protease with an activity-based probe compound comprising a sulfoxonium ylide moiety as warhead, and
(2) subsequently analyzing the cysteine protease comprising measuring a detectable signal. In certain such embodiments, the method is an in vitro method.

In certain embodiments, the present invention is directed to an in vitro method of detecting cysteine protease activity comprising
(1) contacting a biological sample with an activity-based probe compound comprising a sulfoxonium ylide moiety as warhead, and
(2) subsequently analyzing the biological sample comprising measuring a detectable signal.

In certain embodiments, the present invention is directed to a method of detecting cysteine protease activity in a biological sample obtained from a subject comprising
(1) contacting the biological sample in vitro with an activity-based probe compound comprising a sulfoxonium ylide moiety as warhead, and
(2) subsequently analyzing the biological sample comprising measuring a detectable signal.

In certain embodiments of the above methods, the activity-based probe compound comprises a sulfoxonium ylide moiety having the formula (IV)

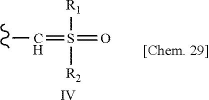

wherein R₁ is selected from the group consisting of (C₁-C₈) alkyl, (C₁-C₈) hydroxyalkyl, (C₁-C₈) haloalkyl, (C₃-C₈) cycloalkyl, (C₂-C₈) alkenyl and (C₂-C₈) alkynyl; and R₂ is selected from the group consisting of (C₁-C₈) alkyl, (C₁-C₈) hydroxyalkyl, (C₁-C₈) haloalkyl, (C₃-C₈) cycloalkyl, (C₂-C₈) alkenyl and (C₂-C₈) alkynyl. In certain such embodiments, R₁ is (C₁-C₈) alkyl, and R₂ is (C₁-C₈). In certain such embodiments, R₁ is methyl and R₂ is methyl.

In certain embodiments, the present invention is directed to a method of detecting cysteine protease activity comprising
(1) contacting the cysteine protease with a compound of formula I as described herein or a salt thereof, or with a composition as described herein, and
(2) subsequently analyzing the cysteine protease comprising measuring a detectable signal. In certain such embodiments, the method is an in vitro method.

In certain embodiments, the present invention is directed to an in vitro method of detecting cysteine protease activity comprising
(1) contacting a biological sample with a compound of formula I as described herein or a salt thereof, or with a composition as described herein, and
(2) subsequently analyzing the biological sample comprising measuring a detectable signal.

In certain embodiments, the present invention is directed to a method of detecting cysteine protease activity in a biological sample obtained from a subject comprising
(1) contacting the biological sample in vitro with a compound of formula I as described herein or a salt thereof, or with a composition as described herein, and
(2) subsequently analyzing the biological sample comprising measuring a detectable signal.

In certain embodiments of each of the above methods, step (2) comprises performing at least one analytical method selected from the group consisting of gel electrophoresis and subsequent in-gel fluorescence, gel electrophoresis and subsequent radiography, gel electrophoresis and subsequent immunoblotting, fluorescent microscopy, flow cytometry, ex vivo optical imaging, radiography, affinity purification and subsequent mass spectrometry, and affinity purification and subsequent proteomics.

In certain embodiments of each of the above methods, the detectable signal is measured by fluorescence measurement. In certain such embodiments, the at least one analytical method is selected from the group consisting of gel electrophoresis and subsequent in-gel fluorescence, fluorescent microscopy, and flow cytometry. In certain such embodiments, the at least one analytical method is selected from gel electrophoresis and subsequent in-gel fluorescence, and fluorescent microscopy. In certain such embodiments, said compound comprises a detectable element in the form of a fluorescent label (e.g., as described herein). In certain other embodiments, said compound comprises a detectable element in the form of a bioorthogonal ligation handle, and step (2) comprises secondary labeling by click-chemistry to apply a fluorescent label prior to performing the at least one analytical method. In certain other embodiments, said compound comprises a detectable element in the form of biotin, and step (2) comprises secondary labeling with fluorescently tagged streptavidin or secondary labeling with a fluorescently tagged antibody specific for biotin, prior to performing the at least one analytical method.

In certain embodiments of each of the above methods, the at least one analytical method is selected from the group consisting of radiography, and gel electrophoresis and subsequent radiography. In certain such embodiments, said compound comprises a detectable element in the form of a radiolabel. In certain other embodiments, said compound comprises a detectable element in the form of a chelator for a radiolabel. In certain other embodiments, said compound comprises a detectable element in the form of a bioorthogonal ligation handle, and step (2) comprises secondary labeling by click-chemistry to apply a radiolabel or a chelator for a radiolabel prior to performing the at least one analytical method.

In certain embodiments of each of the above methods, the at least one analytical method is selected from the group consisting of affinity purification and subsequent mass spectrometry, and affinity purification and subsequent proteomics. In certain such embodiments, said compound comprises a detectable element in the form of a biotin label. In certain other embodiments, said compound comprises a detectable element in the form of a bioorthogonal ligation handle, and step (2) comprises secondary labeling by click-chemistry to apply a biotin label prior to performing the at least one analytical method. In the case of biotin-labeling, affinity purification can be performed using, e.g., streptavidin-coated beads, or beads coated with an antibody specific for biotin.

In certain embodiments, the affinity purification can be performed using beads coated with an antibody specific for a certain tag. In certain such embodiments, said compound comprises said tag as detectable element. In certain other embodiments, said compound comprises a detectable element in the form of a bioorthogonal ligation handle, and step (2) comprises secondary labeling by click-chemistry to apply said tag prior to performing the affinity purification.

In certain embodiments of each of the above methods, the at least one analytical method is selected from the group consisting of gel electrophoresis and subsequent immunoblotting. In certain such embodiments, said compound comprises a detectable element in the form of a biotin label, and step (2) comprises secondary labeling, e.g., with HRP-tagged-streptavidin prior to performing the at least one analytical method. In certain other embodiments, said compound comprises a detectable element in the form of a bioorthogonal ligation handle, and step (2) comprises secondary labeling by click-chemistry to apply a biotin label and subsequent labeling, e.g., with HRP-tagged-streptavidin prior to performing the at least one analytical method.

In certain embodiments, the gel electrophoresis is a one-dimensional or a two-dimensional gel electrophoresis (such as SDS-Page or native PAGE). In certain embodiments, the gel electrophoresis is an SDS-PAGE.

### In Vitro Methods of Detection With Prior Administration to Subject

In certain embodiments, the present invention is directed to a method of detecting cysteine protease activity comprising
(1) administering to a subject an activity-based probe compound comprising a sulfoxonium ylide moiety as warhead,
(2) subsequently obtaining a biological sample from the subject; and
(3) subsequently analyzing the biological sample comprising measuring a detectable signal.

In certain such embodiments, the activity-based probe compound is administered intravenously. In certain embodiments of this method, the activity-based probe compound comprises a sulfoxonium ylide moiety having the formula (IV)

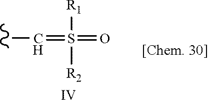

wherein
R₁ is selected from the group consisting of (C₁-C₈) alkyl, (C₁-C₈) hydroxyalkyl, (C₁-C₈) haloalkyl, (C₃-C₈) cycloalkyl, (C₂-C₈) alkenyl and (C₂-C₈) alkynyl; and
R₂ is selected from the group consisting of (C₁-C₈) alkyl, (C₁-C₈) hydroxyalkyl, (C₁-C₈) haloalkyl, (C₃-C₈) cycloalkyl, (C₂-C₈) alkenyl and (C₂-C₈) alkynyl. In certain such embodiments, R₁ is (C₁-C₈) alkyl, and R₂ is (C₁-C₈). In certain such embodiments, R₁ is methyl and R₂ is methyl.

In certain embodiments, the present invention is directed to a method of detecting cysteine protease activity comprising
(1) administering to a subject a compound of formula I as described herein or a salt thereof, or a composition as described herein,
(2) subsequently obtaining a biological sample from the subject; and
(3) subsequently analyzing the biological sample comprising measuring a detectable signal. In certain such embodiments, the compound or salt thereof, or the composition, is administered intravenously.

In certain embodiments of each of the above methods, step (3) comprises performing at least one analytical method selected from the group consisting of gel electrophoresis and subsequent in-gel fluorescence, gel electrophoresis and subsequent radiography, gel electrophoresis and subsequent immunoblotting, fluorescent microscopy, flow cytometry, ex vivo optical imaging, radiography, affinity purification and subsequent mass spectrometry, and affinity purification and subsequent proteomics.

In certain embodiments of each of the above methods, the detectable signal is measured by fluorescence measurement. In certain such embodiments, the at least one analytical method is selected from the group consisting of gel electrophoresis and subsequent in-gel fluorescence, fluorescent microscopy, and flow cytometry. In certain such embodiments, the at least one analytical method is selected from gel electrophoresis and subsequent in-gel fluorescence, and fluorescent microscopy. In certain such embodiments, said compound comprises a detectable element in the form of a fluorescent label (e.g., as described herein). In certain other embodiments, said compound comprises a detectable element in the form of a bioorthogonal ligation handle, and step (3) comprises secondary labeling by click-chemistry to apply a fluorescent label prior to performing the at least one analytical method. In certain other embodiments, said compound comprises a detectable element in the form of biotin, and step (3) comprises secondary labeling with fluorescently tagged streptavidin or secondary labeling with a fluorescently tagged antibody specific for biotin, prior to performing the at least one analytical method.

In certain embodiments of each of the above methods, the at least one analytical method is selected from the group consisting of radiography, and gel electrophoresis and subsequent radiography. In certain such embodiments, said compound comprises a detectable element in the form of a radiolabel. In certain other embodiments, said compound comprises a detectable element in the form of a chelator for a radiolabel. In certain other embodiments, said compound comprises a detectable element in the form of a bioorthogonal ligation handle, and step (3) comprises secondary labeling by click-chemistry to apply a radiolabel or a chelator for a radiolabel prior to performing the at least one analytical method.

In certain embodiments of each of the above methods, the at least one analytical method is selected from the group consisting of affinity purification and subsequent mass spectrometry, and affinity purification and subsequent proteomics. In certain such embodiments, said compound comprises a detectable element in the form of a biotin label. In certain other embodiments, said compound comprises a detectable element in the form of a bioorthogonal ligation handle, and step (3) comprises secondary labeling by click-chemistry to apply a biotin label prior to performing the at least one analytical method. In the case of biotin-labeling, affinity purification can be performed using, e.g., streptavidin coated beads, or beads coated with an antibody specific for biotin. In certain embodiments, the affinity purification can be performed using beads coated with an antibody specific for a certain tag. In certain such embodiments, said compound comprises said tag as detectable element. In certain other embodiments, said compound comprises a detectable element in the form of a bioorthogonal ligation handle, and step (3) comprises secondary labeling by click-chemistry to apply said tag prior to performing the affinity purification.

In certain embodiments of each of the above methods, the at least one analytical method is selected from the group consisting of gel electrophoresis and subsequent immunoblotting. In certain such embodiments, said compound comprises a detectable element in the form of a biotin label, and step (3) comprises secondary labeling, e.g., with HRP-tagged-streptavidin prior to performing the at least one analytical method. In certain other embodiments, said compound comprises a detectable element in the form of a bioorthogonal ligation handle, and step (3) comprises secondary labeling by click-chemistry to apply a biotin label and subsequent labeling, e.g., with HRP-tagged-streptavidin prior to performing the at least one analytical method.

In certain embodiments, the gel electrophoresis is a one-dimensional or a two-dimensional gel electrophoresis (such as SDS-Page or native PAGE). In certain embodiments, the gel electrophoresis is an SDS-PAGE. In certain embodiments, the subject is a human subject.

### In Vivo Methods of Detection

In certain embodiments, the present invention is directed to an in vivo method of detecting cysteine protease activity in a subject comprising
(1) administering to the subject an activity-based probe compound comprising a sulfoxonium ylide moiety as warhead, and
(2) subsequently examining the subject comprising measuring a detectable signal.

In certain such embodiments, the activity-based probe compound is administered intravenously. In certain embodiments of this method, the activity-based probe compound comprises a sulfoxonium ylide moiety having the formula (IV)

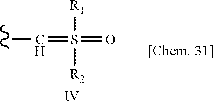

wherein
R₁ is selected from the group consisting of (C₁-C₈) alkyl, (C₁-C₈) hydroxyalkyl, (C₁-C₈) haloalkyl, (C₃-C₈) cycloalkyl, (C₂-C₈) alkenyl and (C₂-C₈) alkynyl; and
R₂ is selected from the group consisting of (C₁₋C₈) alkyl, (C₁-C₈) hydroxyalkyl, (C₁-C₈) haloalkyl, (C₃-C₈) cycloalkyl, (C₂-C₈) alkenyl and (C₁-C₈) alkynyl. In certain such embodiments, R₁ is (C₁-C₈) alkyl, and R₂ is (C₁-C₈). In certain such embodiments, R₁ is methyl and R₂ is methyl.

In certain embodiments, the present invention is directed to an in vivo method of detecting cysteine protease activity in a subject comprising
(1) administering to the subject a compound of formula I as described herein or a salt thereof, or a composition as described herein, and
(2) subsequently examining the subject comprising measuring a detectable signal.

In certain such embodiments, the compound or salt thereof, or the composition, is administered intravenously.

In certain embodiments of each of the above (in vivo) methods, the detectable signal is measured by in vivo optical imaging, radiography, or positron emission tomography. In certain such embodiments the detectable signal is measured by radiography, or positron emission tomography. In certain such embodiments, said compound comprises a detectable element in the form of a radiolabel. In certain other embodiments, said compound comprises a detectable element in the form of a chelator for a radiolabel. In certain embodiments, the subject is a human subject.

### Methods of Inhibiting Cysteine Proteases

In certain embodiments, the present invention is directed to a method of inhibiting a cysteine protease comprising contacting the cysteine protease with a compound of formula I as described herein or a salt thereof, or with a composition as described herein. In certain such embodiments, the method is an in vitro method.

In certain embodiments, the present invention is directed to an in vitro method of inhibiting a cysteine protease comprising contacting a biological sample with a compound of formula I as described herein or a salt thereof, or with a composition as described herein.

In certain embodiments, the present invention is directed to a method of inhibiting a cysteine protease in a biological sample obtained from a subject comprising contacting the biological sample in vitro with a compound of formula I as described herein or a salt thereof, or with a composition as described herein.

In certain embodiments, the present invention is directed to an in vivo method of inhibiting a cysteine protease in a subject comprising administering to the subject a compound of formula I as described herein or a salt thereof, or a composition as described herein. In certain such embodiments, the compound or salt thereof, or the composition, is administered intravenously.

In certain embodiments of the methods of inhibiting a cysteine protease as described herein, the compound of formula I as described herein does not contain a detectable element such as a fluorescent label, a biotin label, a radiolabel, a chelator, and a bioorthogonal ligation handle.

### Cysteine Proteases

In the methods of detecting cysteine protease activity as described herein, and in the methods of inhibiting a cysteine protease as described herein, the cysteine protease can for example be a mammalian cysteine protease. In certain embodiments, the cysteine protease is a human cysteine protease. In certain embodiments, the cysteine protease is a cysteine cathepsin. In certain embodiments, the cysteine protease is a mammalian cysteine cathepsin. In certain embodiments, the cysteine protease is a human cysteine cathepsin. In certain embodiments, the cysteine protease is cathepsin S and/or cathepsin X. In certain embodiments, the cysteine protease is cathepsin X. In certain embodiments, the cysteine protease is mammalian cathepsin X. In certain embodiments, the cysteine protease is human cathepsin X.

In certain embodiments of the methods of detecting cysteine protease activity as described herein, cathepsin X activity is detected and cathepsin B activity and/or cathepsin L activity are not detected. In certain embodiments, cathepsin X activity and cathepsin S activity are detected and cathepsin B activity and/or cathepsin L activity are not detected.

In certain embodiments of the methods of inhibiting a cysteine protease as described herein, cathepsin X is inhibited and cathepsin B and/or cathepsin L are not inhibited. In certain embodiments, cathepsin X and cathepsin S are inhibited and cathepsin B and/or cathepsin L are not inhibited.

In the in vitro methods of detecting cysteine protease activity as described herein, the identity of the labelled protein can be verified, e.g., by subjecting an aliquot of the probelabelled sample to an immunoprecipitation test (e.g., a pulldown with an antibody specific for the respective cysteine protease (e.g., cathepsin X).

### Methods Of Diagnosis And Respective Compounds/Compositions for Use in Diagnosis

In certain embodiments, the present invention is directed to a method of diagnosing a disease associated with a cysteine protease activity in a subject comprising
(1) contacting a biological sample obtained from the subject in vitro with a compound of formula I as described herein or a salt thereof, or with a composition as described herein, and
(2) subsequently analyzing the biological sample comprising measuring a detectable signal. In certain such embodiments, the method comprises detecting cysteine protease activity according to any of the methods described herein in the section “(In vitro) methods of detection”.

In certain embodiments, the present invention is directed to a method of diagnosing a disease associated with a cysteine protease activity in a subject comprising
(1) administering to the subject a compound of formula I as described herein or a salt thereof, or a composition as described herein,
(2) subsequently obtaining a biological sample from the subject; and
(3) subsequently analyzing the biological sample comprising measuring a detectable signal.

In certain such embodiments, the method comprises detecting cysteine protease activity according to any of the methods described herein in the section “(In vitro) methods of detection with prior administration to subject”.

In certain embodiments, the present invention is directed to an in vivo method of diagnosing a disease associated with a cysteine protease activity in a subject comprising
(1) administering to the subject a compound of formula I as described herein or a salt thereof, or a composition as described herein, and
(2) subsequently examining the subject comprising measuring a detectable signal.

In certain such embodiments, the method comprises detecting cysteine protease activity according to any of the methods described herein in the section” “In vivo methods of detection”.

In certain embodiments, the present invention is directed to a compound of formula I as described herein or a salt thereof, for use in a method of diagnosing a disease associated with a cysteine protease activity in a subject, wherein the method comprises
(1) administering to the subject a compound of formula I as described herein or a salt thereof,
(2) subsequently obtaining a biological sample from the subject; and
(3)subsequently analyzing the biological sample comprising measuring a detectable signal.

In certain such embodiments, the method comprises detecting cysteine protease activity according to any of the methods described herein in the section “(In vitro) methods of detection with prior administration to subject”.

In certain embodiments, the present invention is directed to a composition as described herein for use in a method of diagnosing a disease associated with a cysteine protease activity in a subject, wherein the method comprises
(1) administering to the subject a composition as described herein,
(2) subsequently obtaining a biological sample from the subject; and
(3) subsequently analyzing the biological sample comprising measuring a detectable signal.

In certain such embodiments, the method comprises detecting cysteine protease activity according to any of the methods described herein in the section “(In vitro) methods of detection with prior administration to subject”.

In certain embodiments, the present invention is directed to a compound of formula I as described herein or a salt thereof, for use in an in vivo method of diagnosing a disease associated with a cysteine protease activity in a subject, wherein the method comprises
(1) administering to the subject a compound of formula I as described herein or a salt thereof, and
(2) subsequently examining the subject comprising measuring a detectable signal.

In certain such embodiments, the method comprises detecting cysteine protease activity according to any of the methods described herein in the section“In vivo methods of detection”.

In certain embodiments, the present invention is directed to a composition as described herein for use in an in vivo method of diagnosing a disease associated with a cysteine protease activity in a subject wherein the method comprises
(1) administering to the subject a composition as described herein, and
(2) subsequently examining the subject comprising measuring a detectable signal.

In certain such embodiments, the method comprises detecting cysteine protease activity according to any of the methods described herein in the section” “In vivo methods of detection”.

### Biological Samples

In certain embodiments, the biological sample is selected from the group consisting of cells, cell lysates, tissue samples, tissue lysates and bodily fluids. In certain embodiments, the biological sample is a cell lysate or a tissue lysate, such as a cleared cell lysate or a cleared tissue lysate. In certain embodiments, the biological sample is live cells. In certain embodiments of a method of detection as described above, the live cells are lysed and cleared between step (1) and step (2), and between step (2) and (3), respectively. In certain embodiments, the biological sample is obtained from a human subject.

In certain embodiments, the biological sample is obtained from the oral cavity (such as the oral mucosa), the lung, the brain, the spinal cord, the pancreas, the stomach, the prostate, the liver, the bone marrow, the colon (such as the distal colon or the proximal colon), the rectum, the breast, the skin, a mucosa or mucus of a subject, or from the feces or sputum of a subject (such as a mammal, and more specifically a human subject). The biological sample can be tumor tissue obtained from the oral cavity, the lung, the brain, the spinal cord, the pancreas, the stomach, the prostate, the liver, the bone marrow, the colon (such as the distal colon or the proximal colon), the rectum, the breast, the skin, or a mucosa or mucus of a subject.

In certain such embodiments, the biological sample is obtained from the oral cavity (such as the oral mucosa) of a subject. In certain such embodiments the biological sample is a cell lysate or a tissue lysate. In certain embodiments the biological sample is an oral biopsy. In certain embodiments the biological sample is an oral mucosal biopsy.

In certain embodiments, the biological sample is obtained from the gastro-intestinal tract of a subject. In certain such embodiments the biological sample is a cell lysate or a tissue lysate. In certain such embodiments the biological sample is an oral biopsy, an esophagus sample, a stomach sample, a small intestine sample, a colon sample, a proximal colon sample, a distal colon sample, a rectal sample, a fecal sample, or a mucosal biopsy. In certain embodiments wherein the biological sample is obtained from the gastro-intestinal tract of a subject, the biological sample is a mucosal biopsy selected from the group consisting of an oral mucosal biopsy, an esophagus mucosal biopsy, a small intestine mucosal biopsy, a colon mucosal biopsy, or a rectal mucosal biopsy.

In certain embodiments, the biological sample is obtained from the colon (such as the distal colon or the proximal colon) of a subject. In certain such embodiments the biological sample is a cell lysate or a tissue lysate. In certain embodiments the biological sample is a colon biopsy. In certain embodiments the biological sample is a colon mucosal biopsy. In certain embodiments wherein the biological sample is obtained from the colon, the biological sample is a fecal sample.

In certain embodiments, the biological sample is obtained from the prostate of a subject. In certain such embodiments the biological sample is a cell lysate or a tissue lysate. In certain embodiments the biological sample is a prostate biopsy.

In certain embodiments, the biological sample can be cells, cell lysates, tissue samples, and tissue lysates obtained from the breast of a female subject (in particular a female human subject).

In certain embodiments the biological sample is a tissue lysate selected from the group consisting of an oral biopsy, a lung sample, a brain sample, a spinal cord sample, a pancreas sample, a stomach sample, a prostate sample, a liver sample, a bone marrow sample, a colon sample (such as a distal colon sample, and a proximal colon sample), a rectal sample, a breast sample, a skin sample, a mucosal biopsy, a fecal sample, a sputum sample, and a tumor sample.

### Methods Of Treatment and Respective Compounds/Compositions for Use in Treatment

In certain embodiments, the present invention is directed to a method of treating a disease associated with a cysteine protease activity comprising administering to a patient in need thereof a therapeutically effective amount of a compound of formula I as described herein, or a therapeutically effective amount of a composition as described herein.

In certain embodiments, the present invention is directed to a compound of formula I as described herein for use in the treatment of a disease associated with a cysteine protease activity.

In certain embodiments, the present invention is directed to a composition for use in the treatment of a disease associated with a cysteine protease activity comprising a compound of formula I as described herein and a carrier.

In certain embodiments, the present invention is directed to a use of a compound of formula I as described herein in the manufacture of a medicament for the treatment of a disease associated with a cysteine protease activity.

In certain embodiments, the present invention is directed to a use of a composition as described herein in the manufacture of a medicament for the treatment of a disease associated with a cysteine protease activity.

In certain of the above embodiments, the compound of formula I as described herein, when administered or used (or for use) in the treatment of a disease, does not contain a detectable element such as a fluorescent label, a biotin label, a radiolabel, a chelator, and a bioorthogonal ligation handle.

### Diseases

In certain embodiments, the disease (to be diagnosed or to be treated) associated with a cysteine protease activity is selected from the group consisting of celiac disease, a gastrointestinal motility disorder, pain, itch, a skin disorder (such as topic dermatitis), dietinduced obesity, a metabolic disorder (including, but not limited to nonalcoholic steatohepatitis (NASH), hepatic and pancreatic disease), asthma, rheumatoid arthritis, periodontitis, an inflammatory disease (including an inflammatory GI disorder, such as an inflammatory bowel disease), a functional GI disorder (such as irritable bowel syndrome, functional chest pain, functional dyspepsia, nausea and vomiting disorder, functional constipation, functional diarrhea, fecal incontinence, functional anorectal pain, and a functional defecation disorder), cancer, a fibrotic disease, a metabolic dysfunction, a neurological disease, and a neurodegenerative disease.

In certain embodiments, the disease (to be diagnosed or to be treated) associated with a cysteine protease activity is selected from the group consisting of cancer, an inflammatory disease and a neurodegenerative disease.

In certain embodiments, the disease (to be diagnosed or to be treated) is a cancer selected from the group consisting of breast cancer, brain cancer (glioblastoma), bone marrow cancer, pancreatic cancer, lung cancer, prostate cancer, liver cancer (hepatic cell carcinoma), oral cancer, colorectal cancer and gastric cancer.

In certain embodiments, the disease (to be diagnosed or to be treated) is an inflammatory disease selected from the group consisting of an inflammatory GI disorder, pancreatitis, and an infection. In certain such embodiments, the inflammatory GI disorder is selected from the group consisting of an inflammatory bowel disease, infectious diarrhea, mesenteric ischaemia, diverticulitis and necrotizing enterocolitis (NEC). In certain embodiments the inflammatory GI disorder is an inflammatory bowel disease. In certain embodiments the inflammatory bowel disease is selected from the group consisting of ulcerative colitis, Crohn’s disease, diversion colitis, indeterminate colitis and pouchitis, microscopic colitis, immuno-oncology colitis, chemotherapy/radiation colitis, Graft versus Host Disease (GvHD) colitis, acute colitis, Beh&ccedil;et’s disease, collagenous colitis, lymphocytic colitis. In certain embodiments the inflammatory GI disorder is selected from ulcerative colitis and Crohn’s disease.

In certain embodiments, the disease (to be diagnosed or to be treated) is an inflammatory disease selected from the group consisting of ulcerative colitis, Crohn’s disease, diversion colitis, indeterminate colitis and pouchitis, microscopic colitis, immuno-oncology colitis, chemotherapy/radiation colitis, Graft versus Host Disease (GvHD) colitis, acute colitis, Beh&ccedil;et’s disease, collagenous colitis, lymphocytic colitis, infectious diarrhea, mesenteric ischaemia, diverticulitis and necrotizing enterocolitis (NEC), pancreatitis, and infections.

In certain embodiments, the disease (to be diagnosed or to be treated) is an inflammatory disease selected from inflammatory bowel diseases. In certain such embodiments, the inflammatory bowel disease is ulcerative colitis or Crohn’s disease.

In certain embodiments, the disease (to be diagnosed or to be treated) is a neurodegenerative disease selected from the group consisting of Alzheimer’s disease, multiple sclerosis, and neuropathic pain.

In certain embodiments relating to the diagnosis of a disease, wherein the disease is breast cancer, the biological sample is a sample as described above which is obtained from the breast of a subject, e.g. from tumor tissue located in the breast.

In certain embodiments relating to the diagnosis of a disease, wherein the disease is brain cancer, the biological sample is a sample as described above which is obtained from the brain of a subject, e.g. from tumor tissue located in the brain.

In certain embodiments relating to the diagnosis of a disease, wherein the disease is bone marrow cancer, the biological sample is a sample as described above which is obtained from the bone marrow of a subject, e.g. from tumor tissue located in the bone marrow.

In certain embodiments relating to the diagnosis of a disease, wherein the disease is pancreatic cancer, the biological sample is a sample as described above which is obtained from the pancreas of a subject, e.g. from tumor tissue located in the pancreas.

In certain embodiments relating to the diagnosis of a disease, wherein the disease is lung cancer, the biological sample is a sample as described above which is obtained from the lung of a subject, e.g. from tumor tissue located in the lung.

In certain embodiments relating to the diagnosis of a disease, wherein the disease is prostate cancer, the biological sample is a sample as described above which is obtained from the prostate of a subject, e.g. from tumor tissue located in the prostate.

In certain embodiments relating to the diagnosis of a disease, wherein the disease is liver cancer, the biological sample is a sample as described above which is obtained from the liver of a subject, e.g. from tumor tissue located in the liver.

In certain embodiments relating to the diagnosis of a disease, wherein the disease is oral cancer, the biological sample is a sample as described above which is obtained from the oral cavity of a subject, e.g. from tumor tissue located in the oral cavity.

In certain embodiments relating to the diagnosis of a disease, wherein the disease is colorectal cancer, the biological sample is a sample as described above which is obtained from the colon or rectum of a subject, e.g. from tumor tissue located in the colon or rectum.

In certain embodiments relating to the diagnosis of a disease, wherein the disease is gastric cancer, the biological sample is a sample as described above which is obtained from the stomach of a subject, e.g. from tumor tissue located in the stomach.

In certain embodiments relating to the diagnosis of a disease, wherein the disease is an inflammatory GI disorder or a functional GI disorder, the biological sample is a sample as described above which is obtained from the gastro-intestinal tract (such as the colon) of a subject.

In certain embodiments relating to the diagnosis of a disease, wherein the disease is an inflammatory bowel disease, the biological sample is a sample as described above which is obtained from the gastro-intestinal tract (such as the colon) of a subject.

In certain embodiments relating to the diagnosis of a disease, wherein the disease is ulcerative colitis, Crohn’s disease, diversion colitis, indeterminate colitis and pouchitis, microscopic colitis, immuno-oncology colitis, chemotherapy/radiation colitis, Graft versus Host Disease (GvHD) colitis, acute colitis, Beh&ccedil;et’s disease, collagenous colitis, lymphocytic colitis, infectious diarrhea, mesenteric ischaemia, diverticulitis or necrotizing enterocolitis (NEC), the biological sample is a sample as described above which is obtained from the gastro-intestinal tract (such as the colon) of a subject.

In certain embodiments relating to the diagnosis of a disease, wherein the disease is pancreatitis, the biological sample is a sample as described above which is obtained from the pancreas of a subject.

In certain embodiments relating to the diagnosis of a disease, wherein the disease is an infection, the biological sample is a sample as described above which is obtained from the infected area or body part of a subject.

In certain embodiments relating to the diagnosis of a disease, wherein the disease is Alzheimers disease, the biological sample is a sample as described above which is obtained from the brain of a subject.

In certain embodiments relating to the diagnosis of a disease, wherein the disease is multiple sclerosis, the biological sample is a sample as described above which is obtained from the brain of a subject.

In certain embodiments relating to the diagnosis of a disease, wherein the disease is neuropathic pain, the biological sample is a sample as described above which is obtained from the spinal cord of a subject.

In certain embodiments, the biological sample comprises a sample obtained from pathological tissue of a subject (e.g., tumor tissue, inflamed tissue, and/or infected tissue). In certain embodiments, the biological sample comprises a sample obtained from pathological tissue of a subject, and a sample obtained from normal (non-pathological) tissue of the same subject as a control.

In certain embodiments the disease (to be diagnosed or to be treated) is an oral cancer. In certain such embodiments the oral cancer is an oral squamous cell carcinoma. For the diagnosis of oral cancer such as oral squamous cell carcinoma, the biological sample can be a tissue sample (tissue lysate) obtained from the oral cavity of a subject, e.g. from tumor tissue located in the oral cavity. In certain such embodiments the biological sample is an oral mucosal biopsy obtained from a subject (e.g. including a biopsy from tumor tissue and from normal (non-pathological) tissue of the same subject).

In certain embodiments, the disease associated with a cysteine protease activity is a disease associated with a cysteine protease activity, wherein the cysteine protease is a mammalian cysteine protease. In certain embodiments, the cysteine protease is a human cysteine protease. In certain embodiments, the cysteine protease is a cysteine cathepsin. In certain embodiments, the cysteine protease is a mammalian cysteine cathepsin. In certain embodiments, the cysteine protease is a human cysteine cathepsin. In certain embodiments, the cysteine protease is cathepsin S and/or cathepsin X. In certain embodiments, the cysteine protease is cathepsin X. In certain embodiments, the cysteine protease is mammalian cathepsin X. In certain embodiments, the cysteine protease is human cathepsin X.

In certain embodiments, the disease associated with a cysteine protease activity is a disease associated with cathepsin X activity.

### Synthesis Of Chloromethylketones Via a Sulfoxonium Ylide Intermediate

Many of the reported activity-based probes for cysteine proteases incorporate acyloxymethylketone (AOMK) or phenoxymethylketone (PMK) warheads (Edgington et al., 2009; 2012; 2013; Oresic Bender et al., 2015; Verdoes et al., 2013; 2012). Synthesis of these electrophiles requires generation of chloromethylketone intermediates, a process that has historically been achieved through generation of diazomethane, an extremely explosive yellow gas. The present inventors have now discovered a new synthetic route to access the chloromethylketones via a sulfoxonium ylide intermediate (Scheme 2), which does not require generation of diazomethanes, thereby avoiding this potentially dangerous reaction and thus providing for a safer alternative to the previously used methods. This method also circumvents the need for N-methyl-N-nitroso-p-toluenesulfonamide (Diazald&reg;), a product that has recently been discontinued by Sigma Aldrich.

Thus, in certain embodiments, the present invention relates to a process for preparing a compound bearing a chloromethylketone moiety comprising reacting a compound bearing a sulfoxonium ylide moiety to yield the compound bearing the chloromethylketone moiety.

In certain embodiments, the present invention relates to a process for preparing an activity-based probe compound bearing an acyloxymethylketone moiety or a phenoxymethylketone moiety as warhead, comprising
(i) preparing an intermediate compound bearing a chloromethylketone moiety by reacting a compound bearing a sulfoxonium ylide moiety to yield the compound bearing the chloromethylketone moiety; and
(ii) further processing the compound bearing the chloromethylketone moiety to yield said activity-based probe compound.

In certain of the above embodiments, the process comprises reacting the compound bearing the sulfoxonium ylide moiety with hydrochloric acid to yield the compound bearing the chloromethylketone moiety. In certain such embodiments the compound bearing the sulfoxonium ylide moiety is reacted with hydrochloric acid at elevated temperature. In certain such embodiments the compound bearing the sulfoxonium ylide moiety is reacted with hydrochloric acid at elevated temperature and in an organic solvent. In certain such embodiments the organic solvent comprises an ether such as tetrahydrofuran.

In certain embodiments, the sulfoxonium ylide moiety has the formula (IV)

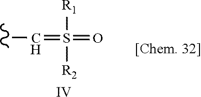

wherein
R₁ is selected from the group consisting of (C₁-C₈) alkyl, (C₁-C₈) hydroxyalkyl, (C₁-C₈) haloalkyl, (C₃-C₈) cycloalkyl, (C₂-C₈) alkenyl and (C₂-C₈) alkynyl; and
R₂ is selected from the group consisting of (C₁-C₈) alkyl, (C₁-C₈) hydroxyalkyl, (C₁-C₈) haloalkyl, (C₁-C₈) cycloalkyl, (C₂-C₈) alkenyl and (C₂-C₈) alkynyl. In certain such embodiments, R₁ is (C₁-C₈) alkyl, and R₂ is (C₁-C₈) alkyl. In certain such embodiments, R₁ is methyl and R₂ is methyl.

Using the above described improved method, three AOMK probes bearing Lys, Phe, and Nle have been successfully generated, suggesting that this method can be broadly applied to the synthesis of diverse ABPs. A comparison of the synthesized AOMK probes with the sulfoxonium ylide probes bearing identical recognition sequences revealed that the respective sulfoxonium ylide probes are more potent and specific than the acyloxymethylketone analogues, which further validates the utility of the new sulfoxonium ylide warhead. Thus, sulfoxonium ylide probes represent a clear advancement in the tools that are available to study the function of cysteine proteases such as cathepsin X.

In a further aspect, the invention relates to a compound having a formula selected from the group of formulas consisting of:

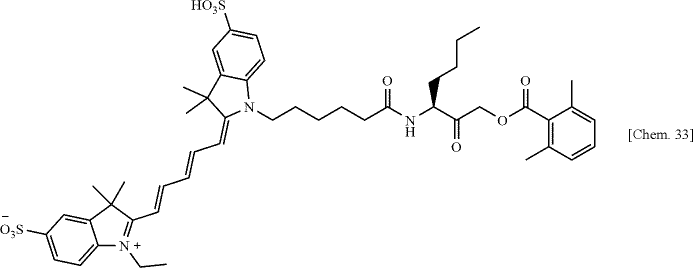

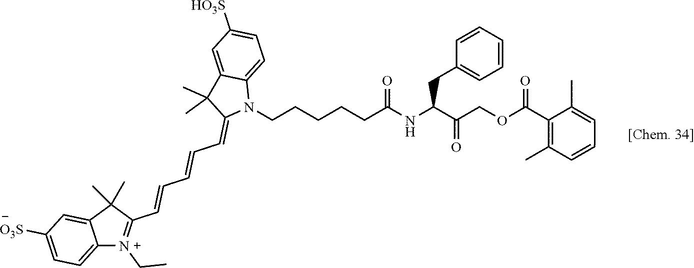

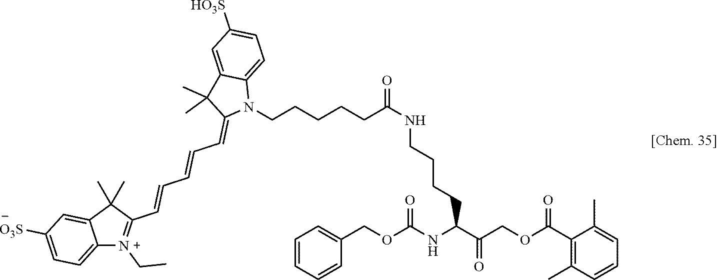

or a salt thereof.

### DETAILED DESCRIPTION OF THE PREFERRED EMBODIMENTS

The present invention is now more fully described with reference to the accompanying examples. It should be understood, however, that the following description is illustrative only and should not be taken in any way as a restriction of the invention.

### Examples

### Synthesis And Characterization Of Compounds

### General Information

All reagents were purchased from commercial suppliers and used without further purification.

Sulfo-Cy5 (free carboxylic acid form) was purchased from from W&J Pharmachem. All solvents used were HPLCgrade. All water-sensitive reactions were performed in anhydrous solvents under argon atmosphere.

RP-HPLCwas performed on a Phenomenex Luna C-8 column (100 Å, 10 µm, 250 × 21.5 mm) utilising a Waters 600 semi-preparative HPLCincorporating a Waters 486 UV detector. The eluting profile was a linear gradient of 0-60% buffer A to buffer B (buffer A: 0.1% TFA in water; buffer B: 0.1% TFA in acetonitrile) over 60 min at a flow rate of 15 mL min-1. Compound identity was confirmed by ESI-MS, using a Shimadzu LCMS2020 instrument, incorporating a Phenomenex Luna C-8 column (100 Å, 3 µm, 100 × 2.00 mm). The eluting profile was 0.1% TFA in water for 4 min, followed by a linear gradient of 0-60% buffer A to buffer B over 10 min, at a flow rate of 0.2 ml min-1.

Nuclear magnetic resonance (NMR) spectra were obtained using a Bruker Avance III Nanobay spectrometer coupled to the BACS 60 automatic sample changer. The spectrometer was equipped with a 5 mm PABBO BB-1H/D Z- GRD probe. ¹HNMRspectra were obtained at 400 MHz. Each resonance was assigned according to the following convention: chemical shift (δ), measured in parts per million (ppm) relative to the residual non-deuterated solvent peak chloroform (unless otherwise specified) as an internal reference relative to trimethylsilane (δ=0), multiplicity, coupling constants (J Hz), number of protons and assignment. Multiplicities are denoted as (s) singlet, (d) doublet, (dd) doublet of doublets, (t) triplet, (q) quartet or (m) multiple.

Carbon nuclear magnetic resonance (¹³CNMR) spectra were obtained at 100 MHz. Each resonance was assigned according to the following convention: chemical shift (δ), measured in parts per million (ppm) relative to the residual non-deuterated solvent peak chloroform (unless otherwise specified) as an internal reference relative to trimethylsilane (δ=0), number of carbons and assignment.

Liquid Chromatography Mass Spectra (LCMS) were conducted on an Agilent UHPLC/MS 1260 instrument (Pump: 1200 Series G1311A Quaternary pump, Autosampler: 1200 Series G1329A Thermostated Autosampler, Detector: 1200 Series (31314B Variable Wavelength Detector). Eluting profile was a linear gradient of 5-100% B over 2.5 min at a flow rate of 0.5 mL/min. Solvent A: water 0.1% formic acid, solvent B: acetonitrile 0.001% formic acid. Liquid chromatography conditions; reverse phase HPLCanalysis, Column: Poroshell 120 EC-C18 3.0 × 50 mm 2.7-micron, column temperature: 35° C., injection volume: 1 uL, Detection: monitored at 254 nm and 214 nm. Mass spectrum conditions ion source: quadrupole. Ion Mode: API-ES, drying gas temp: 350° C. Principle ion peaks (m/z) are reported with intensities of the base peak in brackets.

High resolution mass spectra (HRMS) were conducted on an Agilent 6224 TOF LC/MS Mass Spectrometer coupled to an Agilent 1290 Infinity (Agilent, Palo Alto, CA). All data were acquired and reference mass corrected via a dual-spray electrospray ionisation (ESI) source. Mass Spectrometer Conditions: Ionisation mode: Electrospray Ionisation Drying gas flow: 11 L/min, Solvent A = aqueous 0.1% formic acid, Solvent B = acetonitrile/0.1% formic acid. Found and calculated ion peaks (m/z) are reported.

All analytical HPLCanalyses were done on an Agilent 1260 Infinity Analytical HPLCcoupled with a 1260 Degasser: G1322A, 1260 Binary Pump: G1312B, 1260 HiP ALS autosampler: G1367E, 1260 TCC: G1316A and 1260 DAD detector: G4212B. The column used was a Zorbax Eclipse Plus C18 Rapid Resolution 4.6 × 100 mm 3.5-micron. The sample injection volume was 2 µL, which was run in 0.1% TFA in acetonitrile at a gradient of 5 - 100% over 10 min with a flow rate of 1 mL/min. Detection methods were with 214 nm and 254 nm.

### Example 1: Synthesis Of Sulfoxonium Ylide Compounds

Compounds having a sulfoxonium ylide warhead (sCy5-AA-SY probes) were synthesized according to Scheme 1 and in accordance with the general methods A, B and E described in the following.

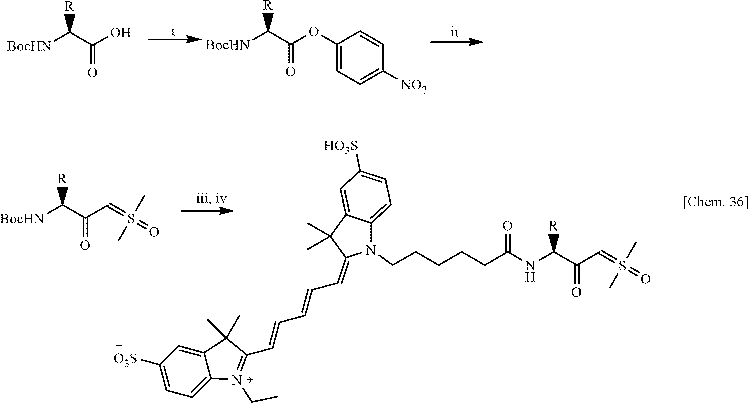

Scheme 1. Synthesis of sCy5-AA-SY probes, i) 4-Nitrophenyichloroformate, EtₐN, DMAP, CH₂Cl₂, 0° C., 6 h. ii) SOMe, KO^{t}Bu, THF, reflux → 0° C.iii) TFA/CH₂Cl₂ (1:1), iv) sulfo-Cy5, PyClock, DIPEA, DMF, r.t, 18 h.

### General Method A: Preparation of Nitrophenyl Esters

Triethylamine (1.2 equiv.) was added to a solution of the Boc-protected amino acid (1 equiv.) in CH₂Cl₂ (5 mL). The stirred mixture was cooled to 0° C. and 4-nitrophenylchloroformate (1.2 equiv.) was added. After 10 min, DMAP (0.1 equiv.) was added and the mixture stirred at 0° C. for 6 h. The reaction mixture was further diluted with CH₂Cl₂ (15 mL) and washed with saturated NaHCO₃ solution (10 mL), 0.1 M HCl solution (10 mL), brine (10 mL), and then dried (MgSO₄), filtered and solvent reduced in vacuo to give the crude product.

### General Method B: Preparation Of Sulfoxonium Ylides

Trimethylsulfoxonium iodide (4 equiv.) was suspended in dry THF (5 mL) and KO^{t}Bu (4 equiv.) added. The mixture was stirred at reflux for 3 h with the exclusion of light. The reaction was cooled to 0° C. and a solution of the nitrophenyl ester (1 equiv.) in THF was added dropwise and stirred for 18 h. The reaction was quenched with H₂O (30 mL) and the solution concentrated in vacuo to remove the THF. The remaining aqueous solution was extracted with EtOAc (3 × 30 mL). The combined organic extract was washed with brine (15 mL), dried (MgSO₄), filtered and the solvent removed in vacuo to give the crude product.

### General Method E: Sulfo-cyanine 5 Labeling Of The Sulfoxonium Ylides

The boc-protected sulfoxonium ylide (1 equiv.) was treated with a 1:1 mixture of TFA and CH₂Cl₂ (2 mL) and stirred at ambient temperature for 1 h. Volatile components were removed under a stream of nitrogen. To the resulting residue was added DMF (300 µL) and DIPEA (4 equiv.). Separately, Sulfo-Cy5 (1 equiv.) and PyClock (2 equiv.) were dissolved in DMF (300 µL) and DIPEA (4 equiv.) and stirred for 2 min before adding to the above solution. The reaction mixture was stirred at ambient temperature for 18 h excluding light.

### Example 1.1: Synthesis and Characterization of Intermediate Nitrophenyl Esters

4-Nitrophenyl (tert-butoxycarbonyl)-L-valinate (SJM-724-20)

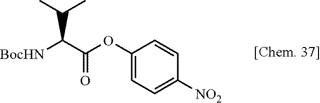 [SMILES: CC(C)[C@H](NC(=O)OC(C)(C)C)C(=O)Oc1ccc([N+](=O)[O-])cc1]

Ice cold dry DMF (20 mL) was slowly added to 4-nitrophenylchloroformate (2.27 g, 11.2 mmol) and stirred for 10 min under a nitrogen atmosphere. The mixture was then gradually warmed to ambient temperature. A solution of boc-L-valine (2.02 g, 9.30 mmol) in DMF (7 mL) was added dropwise followed by triethylamine (1.50 mL, 10.8 mmol). Stirring was continued for 1 h. The reaction mixture was diluted with EtOAc (50 mL) and washed with H₂O (3 × 30 mL) and brine (30 mL). The organic layer was dried (MgSO₄), filtered and concentrated under reduced pressure to give the crude product. Purification by column chromatography (SiO₂, 10-13% EtOAc : Petroleum Spirits) yielded the product as a viscous, colourless oil (2.01 g, 64%). HRMS (ESI⁺): Found: m/z 361.1386 (M + Na)⁺, C₁₆H₂₂N₂NaO₆⁺ requires m/z 361.1370. ¹HNMR(400 MHz, CDCl₃) δ 8.29 - 8.23 (m, 2H), 7.31 - 7.25 (m, 2H), 5.05 (d, J = 8.4 Hz, 1H), 4.44 (dd, J = 8.6, 5.1 Hz, 1H), 2.37 - 2.24 (m, IH), 1.45 (s, 9H), 1.09 (d, J = 6.8 Hz, 3H), 1.03 (d, J = 6.9 Hz, 3H). ¹³CNMR(101 MHz, CDCl₃) δ 170.6 (C), 155.9 (C), 155.3 (C), 145.7 (C), 125.4 (CH), 122.5 (CH), 80.6 (C), 59.1 (CH), 31.2 (CH), 28.4 (CH₃), 19.3 (CH₃), 17.9 (CH₃). LC-MS (ESI⁺) m/z: 339.3 (M + H)⁺ (40%), 360.8 (M + Na)⁺ (80%).

4-Nitrophenyl (tert-butoxycarbonyl)-L-isoleucinate (SJM-724-64)

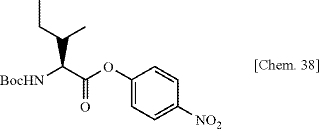 [SMILES: CCC(C)[C@H](NC(=O)OC(C)(C)C)C(=O)Oc1ccc([N+](=O)[O-])cc1]

The title compound was prepared via Method A from boc-L-isoleucine (200 mg, 0.86 mmol), 4-nitrophenylchloroformate (209 mg, 1.04 mmol), DMAP (11 mg, 0.09 mmol) and Et₃N (145 µL, 1.04 mmol) in CH₂Cl₂ to give the crude product as a colourless oil. Purification by column chromatography (SiO₂, Petroleum Spirits : EtOAc 9:1) yielded the product as a white solid (259 mg, 85%). ¹H NMR(401 MHz, CDCl₃) δ 8.31 - 8.25 (m, 2H), 7.33 - 7.27 (m, 2H), 5.03 (d, J = 7.8 Hz, 1H), 4.49 (dd, J = 8.3, 5.2 Hz, 1H), 2.11 - 1.97 (m, 1H), 1.65 - 1.52 (m, 1H), 1.47 (s, 9H), 1.38 - 1.23 (m, 1H), 1.07 (d, J = 6.8 Hz, 3H), 1.00 (t, J = 7.4 Hz, 3H). LC-MS (ESI⁺) m/z: 252.9 (M - Boc + H)⁺ (100%), 374.9 (M + Na)⁺ (60%).

4-Nitrophenyl (tert-butoxycarbonyl)-L-leucinate (SJM-724-76)

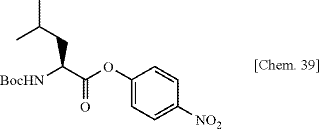 [SMILES: CC(C)C[C@H](NC(=O)OC(C)(C)C)C(=O)Oc1ccc([N+](=O)[O-])cc1]

The title compound was prepared via Method A from boc-L-leucine (200 mg, 0.86 mmol), 4-nitrophenylchloroformate (209 mg, 1.04 mmol), DMAP (11 mg, 0.09 mmol) and Et₃N (145 µL, 1.04 mmol) in CH₂Cl₂, to give the crude product as a yellow oil. Purification by column chromatography (SiO₂, Petroleum Spirits : EtOAc 9:1) yielded the product as a white solid (192 mg, 63%). ¹HNMR(401 MHz, CDCl₃) δ 8.32 - 8.24 (m, 2H), 7.35 - 7.28 (m, 2H), 4.91 (d, J = 7.8 Hz, 1 H), 4.59 - 4.44 (m, 1H), 1.89 - 1.73 (m, 2H), 1.73 - 1.62 (m, 1H), 1.46 (s, 9H), 1.03 (d, J = 2.1 Hz, 3H), 1.02 (d, J = 1.9 Hz, 3H). LC-MS (ESI⁺) m/z: 252.9 (M - Boc + H)⁺ (100%), 374.9 (M + Na)⁺ (55%).

4-Nitrophenyl (S)-2-((tert-butoxycarbonyl)amino)hexanoate (SJM-724-116)

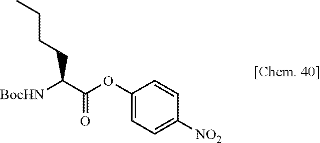 [SMILES: CCCC[C@H](NC(=O)OC(C)(C)C)C(=O)Oc1ccc([N+](=O)[O-])cc1]

The title compound was prepared via Method A from boc-L-norleucine (200 mg, 0.86 mmol), 4-nitrophenylchloroformate (209 mg, 1.04 mmol), DMAP (11 mg, 0.09 mmol) and Et₃N (145 µL, 1.04 mmol) in CH₂Cl₂ to give the crude product as a light yellow solid. Purification by column chromatography (SiO₂, Petroleum Spirits : EtOAc 9:1) yielded the product as a white solid (198 mg, 65%). HRMS (ESI⁺): Found: m/z 375.1534 (M + Na)⁺, C₁₇H₂₄N₂NaO₆⁺ requires m/z 375.1527. ¹HNMR(401 MHz, CDCl₃) δ 8.32 - 8.25 (m, 2H), 7.33 - 7.27 (m, 2H), 4.99 (d, J = 7.3 Hz, 1H), 4.50 (dd, J = 13.0, 7.4 Hz, 1H), 2.02 - 1.91 (m, 1H), 1.86 - 1.74 (m, 1H), 1.51 - 1.35 (m, 13H), 0.95 (t, J = 7.2 Hz, 3H). LC-MS (ESI⁺) m/z: 252.9 (M - Boc + H)⁺ (100%), 374.9 (M + Na)⁺ (95%).

4-Nitrophenyl (tert-butoxycarbonyl)-L-phenylalaninate (SJM-724-68)

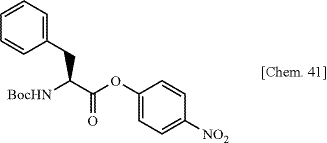 [SMILES: CC(C)(C)OC(=O)N[C@@H](Cc1ccccc1)C(=O)Oc1ccc([N+](=O)[O-])cc1]

The title compound was prepared via Method A from boc-L-phenylalanine (200 mg, 0.75 mmol), 4-nitrophenylchloroformate (182 mg, 0.90 mmol), DMAP (9 mg, 0.08 mmol) and Et₃N (126 µL, 0.90 mmol) in CH₂Cl₂ to give the crude product as a white solid. Purification by column chromatography (SiO₂, CH₂Cl₂) yielded the product as a white solid (251 mg, 86%). ¹H NMR(401 MHz, CDCl₃) δ 8.29 - 8.22 (m, 2H), 7.40 - 7.29 (m, 3H), 7.25 - 7.21 (m, 2H), 7.17 - 7.11 (m, 2H), 5.03 (d, J = 7.0 Hz, 1H), 4.80 (dd, J = 13.6, 6.4 Hz, 1H), 3.31 -3.15 (m, 2H), 1.45 (s, 9H). LC-MS (ESI⁺) m/z: 286.9 (M - Boc + H)⁺ (60%), 408.8 (M + Na)⁺ (40%).

4-Nitrophenyl (tert-butoxycarbonyl)-L-tryptophanate (SJM-724-112)

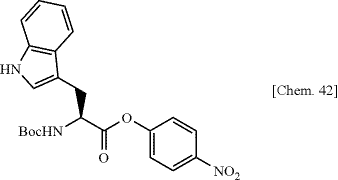 [SMILES: CC(C)(C)OC(=O)N[C@@H](Cc1c[nH]c2ccccc12)C(=O)Oc1ccc([N+](=O)[O-])cc1]

The title compound was prepared via Method A from boc-L-tryptophan (200 mg, 0.66 mmol), 4-nitrophenylchloroformate (159 mg, 0.79 mmol), DMAP (8 mg, 0.07 mmol) and Et₃N (110 µL, 0.79 mmol) in CH₂Cl₂, to give the crude product as a yellow solid. Purification by column chromatography (SiO₂, Petroleum Spirits : EtOAc 2:1) yielded the product as a white solid (200 mg, 72%). ¹H NMR(401 MHz, CDCl₃) δ 8.22 - 8.13 (m, 3H), 7.60 (d, J = 7.9 Hz, 1H), 7.41 (d, J = 8.1 Hz, 1H), 7.25 - 7.22 (m, 1H), 7.16 - 7.11 (m, 2H), 7.03 - 6.98 (m, 2H), 5.15 (d, J = 7.6 Hz, 1H), 4.91 - 4.80 (m, 1H), 3.50 - 3.35 (m, 2H), 1.45 (s, 9H). LC-MS (ESI⁺) m/z: 325.9 (M - Boc + H)⁺ (100%).

4-Nitrophenyl N^{a}-((benzyloxy)carbonyl)-N ^{e}-(tert-butoxycarbonyl)-L-lysinate (SJM-724-40)

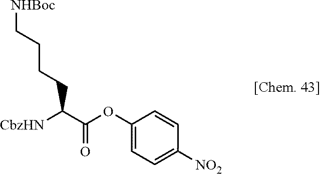 [SMILES: CC(C)(C)OC(=O)NCCCC[C@H](NC(=O)OCc1ccccc1)C(=O)Oc1ccc([N+](=O)[O-])cc1]

Ice cold dry DMF (20 mL) was slowly added to 4-nitrophenylchloroformate (1.27 g, 6.31 mmol) and stirred for 10 min under a nitrogen atmosphere. The mixture was then gradually warmed to ambient temperature. A solution of N^{a}-benzyloxycarbonyl-N ^{e}-(tert-butoxycarbonyl)-L-lysine (2.00 g, 5.26 mmol) in DMF (5 mL) was added dropwise followed by triethylamine (879 µL, 6.31 mmol). Stirring was continued for 20 h. The reaction mixture was diluted with EtOAc (50 mL) and washed with H₂O (4 × 20 mL) and brine (20 mL). The organic layer was dried (MgSO₄), filtered and concentrated under reduced pressure to give the crude product as a yellow oil. Purification by column chromatography (SiO₂, CH₂Cl₂ : EtOAc 95:5) yielded the product as a white solid (672 mg, 25%). ¹H NMR(401 MHz, CDCl₃ δ 8.27 (d, J = 8.9 Hz, 2H), 7.40 - 7.27 (m, 7H), 5.63 - 5.49 (m, 1H), 5.14 (s, 2H), 4.64 - 4.49 (m, 2H), 3.15 (s, 2H), 2.10 - 1.82 (m, 2H), 1.56 - 1.47 (m, 4H), 1.42 (s, 9H). LC-MS (ESI⁺) m/z: 401.9 (M - Boc + H)⁺ (100%), 524.8 (M + Na)⁺ (20%).

(tert-Butoxycarbonyl)-L-phenylalanyl-L-valine (MS-4-178)

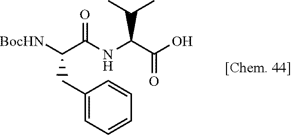 [SMILES: CC(C)[C@H](NC(=O)[C@H](Cc1ccccc1)NC(=O)OC(C)(C)C)C(=O)O]

Fmoc-Val-OH (679 mg, 2.0 mmol) was dissolved in a solution of DCM and triethylamine (842 µL, 6.0 mmol) and added to 2-chlorotrityl resin (1.0 g, 1 mmol) (1 meq/g). The mixture was shaken at ambient temperature for 1 h. The resin was washed with DCM (3 × 5 mL). A solution of MeOH (1 mL) in DCM (5 mL) and triethylamine (0.5 mL) was added to the resin and shaken for 30 min at ambient temperature. Fmoc deprotection was carried out by treatment with 20% piperidine in DMF for 10 minutes and then washed with DMF (3 × 5 mL). Boc-Phe-OH (531 mg, 2.0 mmol) and PyBOP (1.04 g, 2.0 mmol) were dissolved in a solution of DCM and triethylamine (842 µL, 6.0 mmol) and added to the resin. The mixture was shaken at ambient temperature for 1 h and then drained, washed with DCM (3 × 5 mL) and dried under vacuum. The dipeptide was cleaved from the resin by treatment with 20% HFIP in DCM containing 1% TIPS for 2 h. The resin was filtered and the filtrate reduced in vacuo to give the crude product (180 mg).

4-Nitrophenyl (tert-butoxycarbonyl)-L-phenylalanyl-L-valinate (MS-4-182)

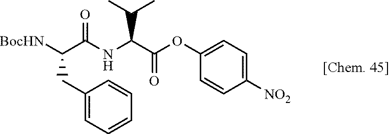 [SMILES: CC(C)[C@H](NC(=O)[C@H](Cc1ccccc1)NC(=O)OC(C)(C)C)C(=O)Oc1ccc([N+](=O)[O-])cc1]

To a solution of 4-nitrophenylchloroformate (116 mg, 0.58 mmol) in dry THF (1 mL) was added dry DMF (150 µL) at 0° C. The reaction was stirred for 10 min under a nitrogen atmosphere before gradually being warmed to ambient temperature. A solution of (tert-butoxycarbonyl)-L-phenylalanyl-L-valine (175 mg, 0.48 mmol) in THF (1 mL) and triethylamine (80 µL) was added dropwise to the above suspension. Stirring was continued for 20 min. The reaction mixture was diluted with EtOAc and washed with H₂O_{.} The organic layer was dried (MgSO₄), filtered and concentrated under reduced pressure to give the crude product (220 mg) as a pale yellow solid.

### Example 1.2. Synthesis and Characterization Of Sulfoxonium Ylide Compounds

tert-butyl (S)-(1-(dimethyl(oxo)-λ⁶-sulfanylidene)-4-methyl-2-oxopentan-3-yl)carbamate (SJM-724-24)

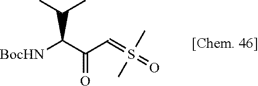 [SMILES: CC(C)[C@H](NC(=O)OC(C)(C)C)C(=O)C=S(C)(C)=O]

The title compound was prepared via Method B from 4-nitrophenyl (tert-butoxycarbonyl)-L-valinate (SJM-724-20) (100 mg, 0.30 mmol). The resulting crude product was purified by column chromatography (SiO₂, EtOAc) and recrystallised from diethyl ether to yield the product as a white solid (58 mg, 67%). HRMS (ESI⁺): Found: m/z 292.1573 (M + H)⁺, C₁₃H₂₆NO₄S⁺ requires m/z 292.1577. ¹H NMR(401 MHz, CDCl₃) δ 5.2.3 (d, J = 8.4 Hz, 1H), 4.50 (s, 1H), 3.94 (dd, J = 8.6, 5.4 Hz, 1H), 3.42 (s, 3H), 3.39 (s, 3H), 2.10 - 1.99 (m, 1H), 1.43 (s, 9H), 0.94 (d, J = 6.7 Hz, 3H), 0.87 (d, J = 6.8 Hz, 3H). ¹³CNMR(101 MHz, CDCl₃) δ 187.89 (C), 155.94 (C), 79.11 (C), 70.07 (CH), 61.89 (CH), 42.25 (CH₃), 42.00 (CH₃), 31.90 (CH), 28.43 (CH₃), 19.60 (CH₃), 17.66 (CH₃). LC-MS (ESI⁺) m/z: 291.9 (M + H)⁺(85%).

tert-butyl ((3S,4S)-1-(dimethyl(oxo)-λ ⁶-sulfanylidene)-4-methyl-2-oxohexan-3-yl)carbamate (SJM-724-92)

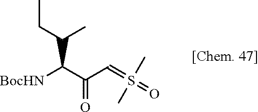 [SMILES: CCC(C)[C@H](NC(=O)OC(C)(C)C)C(=O)C=S(C)(C)=O]

The title compound was prepared via Method B from 4-nitrophenyl (tert-butoxycarbonyl)-L-isoleucitizite (SJM-724-64) (200 mg, 0.57 mmol). The resulting crude product was purified by column chromatography (SiO₂, EtOAc : MeOH, 95:5) to yield the product as a cream solid (126 mg, 73%). HRMS (ESI⁺): Found: m/z 306.1735 (M + H)⁺, C₁₄H₂₈NO₄S⁺ requires m/z 306.1734. ¹HNMR(401 MHz, CDCl₃) δ 5.21 (d, J = 8.9 Hz, 1H), 4.49 (s, 1H), 3.97 (dd, J = 8.7, 5.7 Hz, 1H), 3.41 (s, 3H), 3.38 (s, 3H), 1.83 - 1.73 (m, 1H), 1.53 - 1.46 (m, 1H), 1.43 (s, 9H), 1.18 - 1.04 (m, 1H), 0.91 (d, J = 6.7 Hz, 3H), 0.90 (t, J = 7.5 Hz, 3H). ¹³CNMR(101 MHz, CDCl₃₎ δ 187.8 (C), 155.8 (C), 79.1 (C), 70.0 (CH), 61.3 (CH), 42.4 (CH₃), 42.1 (CH₃), 38.5 (CH), 28.5 (CH₃), 24.9 (CH₂), 15.9 (CH₃), 11.009 (CH₃). LC-MS (ESI⁺) m/z: 305.9 (M + H)⁺ (100%).

tert-butyl (S)-(1-(dimethyl(oxo)-λ ⁶-sulfanylidene)-5-methyl-2-oxohexan-3-yl)carbamate (SJM-724-96)

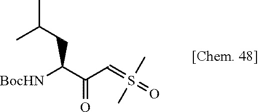 [SMILES: CC(C)C[C@H](NC(=O)OC(C)(C)C)C(=O)C=S(C)(C)=O]

The title compound was prepared via Method B from 4-nitrophenyl (tert-butoxycarbonyl)-L-leucinate (SJM-724-76) (170 mg, 0.48 mmol). The resulting crude product was purified by column chromatography (SiO₂, EtOAc : MeOH, 95:5) to yield the product as a white solid (118 mg, 80%). HRMS (ESI⁺): Found: m/z 306.1735 (M + H)⁺, C₁₄H₂₈NO₄S⁺ requires m/z 306.1734. ¹H NMR(401 MHz, CDCl₃) δ 5.04 (d, J = 8.3 Hz, 1H), 4.51 (s, 1H), 4.16 - 4.06 (m, 1H), 3.41 (s, 3H), 3.38 (s, 3H), 1.75 - 1.62 (m, 2H), 1.62 - 1.50 (m, 1H), 1.43 (s, 9H), 0.94 (d, J = 4.9 Hz, 3H), 0.93 (d, J = 4.9 Hz, 3H). ¹³CNMR(101 MHz, CDCl₃) δ 189.2 (C), 155.6 (C), 79.2 (C), 68.7 (CH), 55.6 (CH), 43.4 (CH₂), 42.4 (CH₃), 42.1 (CH₃), 28.5 (CH₃), 25.0 (CH), 23.2 (CH₃), 22.3 (CH₃). LC-MS (ESI⁺) m/z: 305.9 (M + H)⁺ (80%).

tert-butyl (S)-(1-(dimethyl(oxo)-λ ⁶-sulfanylidene)-2-oxoheptan-3-yl)carbamate (SJM-724-124)

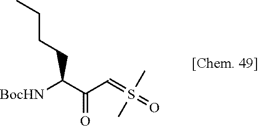 [SMILES: CCCC[C@H](NC(=O)OC(C)(C)C)C(=O)C=S(C)(C)=O]

The title compound was prepared via Method B from 4-nitrophenyl (S)-2-((tert-butoxycarbonyl)amino)hexanoate (SJM-724-116) (170 mg, 0.48 mmol). The resulting crude product was purified by column chromatography (SiO₂, EtOAc : MeOH, 95:5) to yield the product as a white solid (128 mg, 87%). HRMS (ESI⁺): Found: m/z 306.1738 (M + H)⁺, C₁₄H₂₈NO₄S⁺ requires m/z 306.1734. ¹H NMR(401 MHz, CDCl₃) δ 5.19 (d, J = 8.1 Hz, 1H), 4.49 (s, 1H), 4.00 (dd, J = 13.4, 7.5 Hz, 1H), 3.37 (s, 3H), 3.35 (s, 3H), 1.78 - 1.62 (m, 1H), 1.53 - 1.43 (m, 1H), 1.39 (s, 9H), 1.33 - 1.18 (m, 4H), 0.84 (t, J = 7.0 Hz, 3H). ¹³CNMR(101 MHz, CDCl₃) δ 188.6 (C), 155.6 (C), 79.1 (C), 68.9 (CH), 57.0 (CH), 42.3 (CH₃), 42.0 (CH₃), 33.008 (CH₂), 28.4 (CH₃), 27.7 (CH₂), 22.6 (CH₂), 14.0 (CH₃). LC-MS (ESI⁺) m/z: 306.0 (M + H)⁺ (100%).

tert-butyl (S)-(4-(dimethyl(oxo)-λ ⁶-sulfanylidene)-3-oxo-l-phenylbutan-2-yl)carbamate (SJM-724-72)

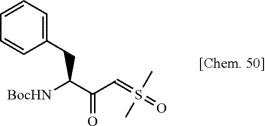 [SMILES: CC(C)(C)OC(=O)N[C@@H](Cc1ccccc1)C(=O)C=S(C)(C)=O]

The title compound was prepared via Method B from 4-nitrophenyl (tert-butoxycarbonyl)-L-phenylalaninate (SJM-724-68) (200 mg, 0.75 mmol). The resulting crude product was purified by column chromatography (SiO₂, CH₂Cl₂) to yield the product as a beige solid (141 mg, 80%). HRMS (ESF): Found: m/z 340.1579 (M + H)⁺, C₁₇H₂₆NO₄S⁺ requires m/z 340.1577. ¹H NWR(401 MHz, CDCl₃) δ 7.29 - 7.17 (m, 5H), 5.22 (d, J = 8.1 Hz, 1H), 4.32 (dd, J = 14.5, 7.004 Hz, 1H), 4.26 (s, 1H), 3.35 (s, 3H), 3.25 (s, 1H), 3.05 - 2.93 (m, 2H), 1.41 (s, 9H). LC-MS (ESI⁺) m/z: 339.9 (M + H)⁺ (100%).

tert-butyl (S)-(4-(dimethyl(oxo)-λ ⁶-sulfanylidene)-1-(1H-indol-3-yl)-3-oxobutan-2-yl)carbamate (SJM-724-120)

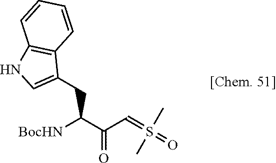 [SMILES: CC(C)(C)OC(=O)N[C@@H](Cc1c[nH]c2ccccc12)C(=O)C=S(C)(C)=O]

The title compound was prepared via Method B from 4-nitrophenyl (tert-butoxycarbonyl)-L-tryptophanate (SJM-724-112) (170 mg, 0.40 mmol). The resulting crude product was purified by column chromatography (SiO₂, EtOAc : MeOH, 95:5) to yield the product as a white solid (118 mg, 78%). HRMS (ESI⁺): Found: m/z 379.1697 (M + H)⁺, C₁₉H₂₇N₂O₄S⁺ requires m/z 379.1686. ¹H NMR (401 MHz, COCl₃) δ 8.44 (s, 1H), 7.63 (d, J = 7.9 Hz, 1H), 7.32 (d, J = 8.1 Hz, 1H), 7.13 (t, J = 7.3 Hz, 1H), 7.08 - 6.98 (m, 2H), 5.40 (d, J = 7.8 Hz, 1H), 4.37 (dd, J = 13.3, 7.1 Hz, 1H), 4.25 (s, 1H), 3.19 (s, 3H), 3.26 - 3.09 (m, 2H), 2.99 (s, 3H), 1.43 (s, 9H). ¹³CNMR(101 MHz, CDCl₃) δ 187.5 (C), 155.5 (C), 136.1 (C), 128.2 (C), 123.0 (CH), 121.9 (CH), 119.3 (CH), 119.2 (CH), 111.7 (C), 111.3 (CH), 79.4 (C), 70.0 (CH), 57.8 (CH), 41.8 (CH₃), 41.6 (CH₃), 29.3 (CH₂), 28.54 (CH₃). LC-MS (ESI⁺) m/z: 378.9 (M + H)⁺ (100%).

benzyl tert-butyl (7-(dimethyl(oxo)-λ⁶-sulfanylidene)-6-oxoheptane-1,5-diyl)(S)-dicarbamate (SJM-724-48)

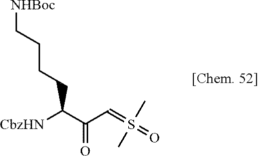 [SMILES: CC(C)(C)OC(=O)NCCCC[C@H](NC(=O)OCc1ccccc1)C(=O)C=S(C)(C)=O]

The title compound was prepared via Method B from 4-Nitrophenyl N^{a}-((benzyloxy)carbonyl)-N^{e}-(tert-butoxycarbonyl)-L-lysinate (SJM-724-40) (200 mg, 0.40 mmol). The resulting crude product was purified by column chromatography (SiO₂, EtOAc : MeOH, 9:1) to yield the product as a white solid (138 mg, 76%). HRMS (ESI⁺): Found: m/z 455.2216 (M + H)⁺, C₂₂H₃₅N₂O₆S⁺ requires m/z 455.2210. ¹H NMR(401 MHz, CDCl₃) δ 7.33 - 7.23 (m, 5H), 5.79 (d, J = 7.9 Hz, 1H), 5.04 (s, 2H), 4.72 (bs, 1H), 4.52 (s, 1H), 4.07 (dd, J = 13.1, 7.6 Hz, 1H), 3.32 (s, 3H), 3.30 (s, 3H), 3.09 - 2.97 (m, 2H), 1.79 - 1.67 (m, 1H), 1.59 - 1.50 (m, 1H), 1.50 - 1.26 (m, 4H), 1.38 (s, 9H). ¹³CNMR(101 MHz, CDCl₃) δ 187.6 (C), 156.1 (C), 136.7 (C), 128.5 (CH), 128.1 (CH), 128.0 (CH), 78.9 (C), 69.5 (CH), 66.6 (CH₂), 57.1 (CH), 42.0 (CH₃), 41.8 (CH₃), 40.3 (CH₂), 33.4 (CH₂), 29.7 (CH₂), 28.4 (CH₃), 22.5 (CH₂). LC-MS (ESI⁺) m/z: 454.9 (M + H)⁺ (100%).

tert-butyl ((S)-1-(((S)-1-(dimethyl(oxo)-λ⁶-sulfaneylidene)-4-methyl-2-oxopentan-3-yl)amino)-1-oxo-3-phenylpropan-2-yl)carbamate (MS-4-186)

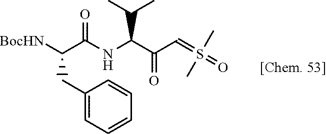 [SMILES: CC(C)[C@H](NC(=O)[C@H](Cc1ccccc1)NC(=O)OC(C)(C)C)C(=O)C=S(C)(C)=O]

The title compound was prepared via Method B from 4-Nitrophenyl (tert-butoxycarbonyl)-L-phenylalanyl-L-valinate (MS-4-182) (200 mg, 0.41 mmol). The resulting crude product was purified by column chromatography (SiO₂, CHCl₂: MeOH 98:2) to yield the title product (68 mg, 38%).

### Example 1.3: Synthesis and Characterization of Sulfoxonium Ylide Probes

sCy5-Val-SY (SJM-724-28)

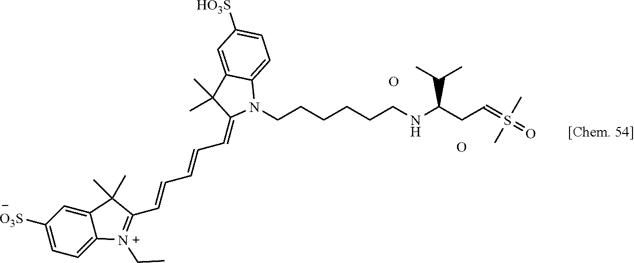

The title compound was prepared via Method E from tert-butyl (S)-(1-(dimethyl(oxo)-λ⁶-sulfanylidene)-4-methyl-2-oxopentan-3-yl)carbamate (SJM-724-24) (5 mg, 0.017 mmol). The resulting crude product was purified by RP-HPLCand lyophilised to yield the product as a blue solid (7.9 mg, 56%). HRMS (ESI⁺): Found: m/z 830.3159 (M + H)⁺, C₄₁H₅₆N₃O₉S₃⁺ requires m/z 830.3173.

sCy5-Ile-SY (SJM-724-104)

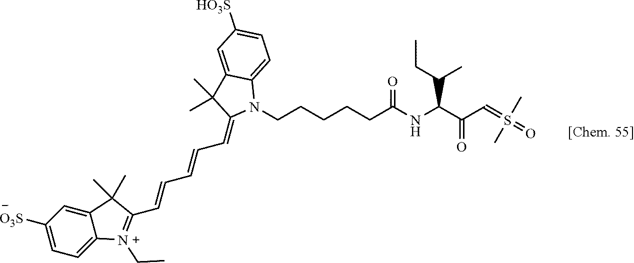

The title compound was prepared via Method E from tert-butyl ((3S,4S)-1-(dimethyl(oxo)-λ⁶-sulfanylidene)-4-methyl-2-oxohexan-3-yl)carbamate (SJM-724-92) (10 mg, 0.033 mmol). The resulting crude product was purified by RP-HPLCand lyophilised to yield the product as a blue solid (3 mg, 11%). HRMS (ESI⁺): Found: m/z 844.3340 (M + H)⁺, C₄₂H₅₈N₃O₉S₃⁺ requires m/z 844.3330.

sCy5-Leu-SY (SJM-724-128)

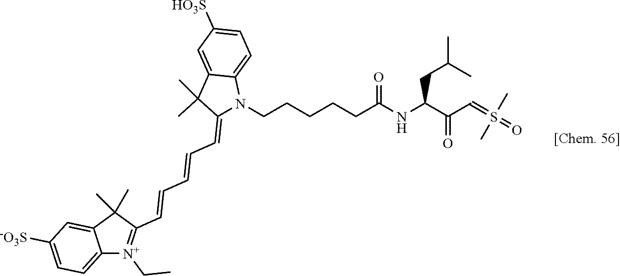

The title compound was prepared via Method E from tert-butyl (S)-(1-(dimethyl(oxo)-λ⁶-sulfanylidene)-5-methyl-2-oxohexan-3-yl)carbamate (SJM-724-96) (10 mg, 0.033 mmol), The resulting crude product was purified by RP-HPLCand lyophilised to yield the product as a blue solid (14.7 mg, 53%). HRMS (ESI⁺): Found: m/z 844.3328 (M + H)⁺, C₄₂H₅₈N₃O₉S₃⁺ requires m/z 844.3330.

sCy5-Nle-SY (SJM-724-132)

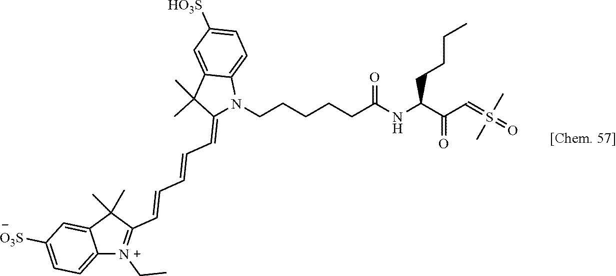

The title compound was prepared via Method E from tert-butyl (S)-(1-(dimethyl(oxo)-λ⁶-sulfanylidene)-2-oxoheptan-3-yl)carbamate (SJM-724-124) (10 mg, 0.033 mmol). The resulting crude product was purified by RP-HPLCand lyophilised to yield the product as a blue solid (17.4 mg, 63%). HRMS (ESI⁺): Found: m/z 844.3335 (M + H)⁺, C₄₂H₅₈N₃O₉S₃⁺ requires m/z 844.3330.

sCy5-Phe-SY (SJM-724-80)

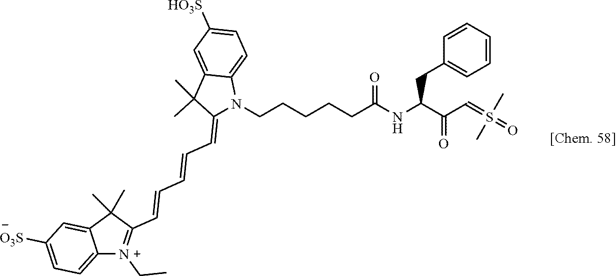

The title compound was prepared via Method E from tert-butyl (S)-(4-(dimethyl(oxo)-λ⁶-sulfanylidene)-3-oxo-1-phenylbutan-2-yl)carbamate (SJM-724-72) (10 mg, 0.029 mmol). The resulting crude product was purified by RP-HPLCand lyophilised to yield the product as a blue solid (13.1 mg, 51%). HRMS (ESI⁺): Found: m/z 878.3157 (M + H)⁺, C₄₅H₅₆N₃O₉S₃⁺ requires m/z 878.3173.

sCy5-Trp-SY (SJM-724-80)

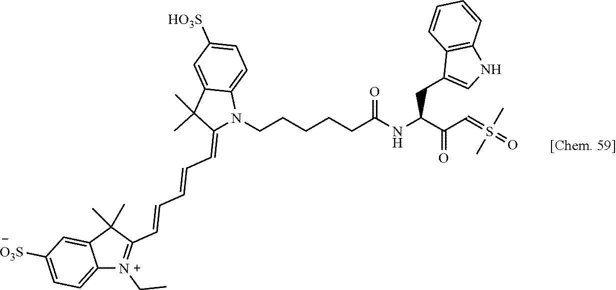

The title compound was prepared via Method E from tert-butyl (S)-(4-(dimethyl(oxo)-λ⁶-sulfanylidene)-1-(1H-indol-3-yl)-3-oxobutan-2-yl)carbamate (SJM-724-120) (10 mg, 0.026 mmol). The resulting crude product was purified by RP-HPLCand lyophilised to yield the product as a blue solid (6 mg, 25%). HRMS (ESI⁺): Found: m/z 917.3277 (M + H)⁺, C₄₇H₅₇N₄O₉S₃⁺ requires m/z 917.3282.

Cbz-Lys(sCy5)-SY (SJM-724-100)

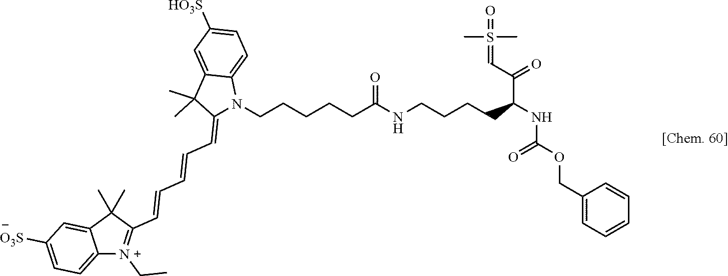

The title compound was prepared via Method E from benzyl tert-butyl (7-(dimethyl(oxo)-λ⁶-sulfanylidene)-6-oxoheptane-1,5-diyl)(S)-dicarbamate (SJM-724-48) (10 mg, 0.022 mmol). The resulting crude product was purified by RP-HPLCand lyophilised to yield the product as a blue solid (10 mg, 46%). HRMS (ESI⁺): Found: m/z 993.3785 (M + H)⁺, C₅₀H₆₅N₄O₁₁S₃⁺ requires m/z 993.3806.

sCy5-Phe-Val-SY (MS-4-191)

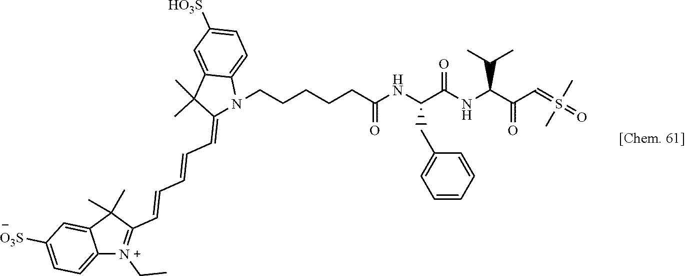

The title compound was prepared via Method E from tert-butyl ((S)-1-(((S)-1-(dimethyl(oxo)-λ⁶-sulfaneylidene)-4-methyl-2-oxopentan-3-yl)amino)-1-oxo-3-phenylpropan-2-yl)carbamate (MS-4-186) (10 mg, 0.022 mmol). The resulting crude product was purified by RP-HPLCand lyophilized to yield the product as a blue solid (4.6 mg, 21%).

### Example 2: Synthesis Of Acyloxymethyl Ketones (AOMK)

Compounds having an acyloxymethyl ketone (AOMK) warhead (sCy5-AA-AOMK probes) were synthesized according to Scheme 2 and in accordance with the general methods C, D and E described in the following.

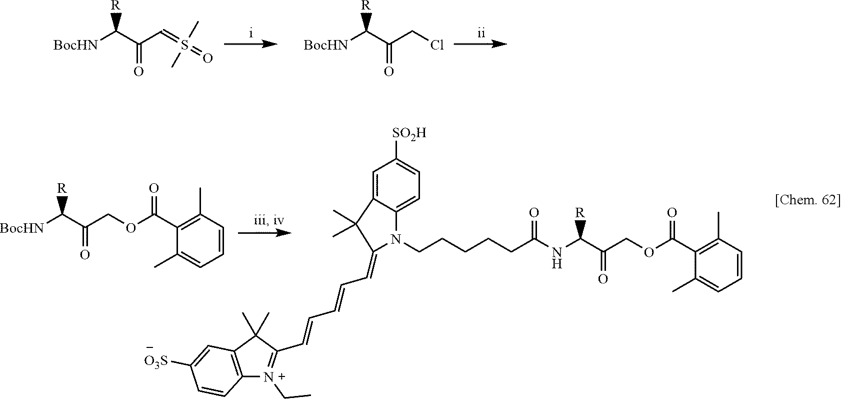

Scheme 2. Synthesis of sCy5-AA-AOMK probes via a sulfoxonium ylide Intermediate, i) 4 M HCl in dioxane, THF, reflux, 4 h. ii) 2,6-dimethyibenzoic acid, KF, DMF, r.t, 18 h. iii) TFA/CH₅Cl₂ (1.1), iv) sulfo-Cy5, PyClock, DIPEA, DMF, r.t, 18 h.

### General Method C: Preparation of Chloromethyl Ketones.

To a solution of the sulfoxonium ylid (1 equiv.) in dry THF (5 mL) was added 4 M HCl in dioxane (1.15 equiv.). The solution was stirred at reflux for 4 h. Solvents were removed in vacuo and the residue treated with EtOAc (20 mL) and washed with H₂O (15 mL) and saturated NaHCO₃ solution (15 mL). The organic layer was dried (MgSO₄), filtered and concentrated in vacuo to give the crude product.

### General Method D: Preparation of Acyloxymethyl Ketones (AOMK).

Potassium Fluoride (3 equiv.) was suspended in DMF (1 mL) and sonicated for 1 min. 2,6-Dimethylbenzoic acid (1.1 equiv.) was added to the suspension and stirred at ambient temperature for 5 min. The chloromethyl ketone (1 equiv.) was added and the mixture stirred at ambient temperature for 18 h. DMF was removed in vacuo and the resulting residue was treated with EtOAc (20 mL) and washed with a saturated NaHCO₃ solution (15 mL). The organic layer was dried (MgSO₄), filtered, and solvent removed in vacuo to give the crude product.

### General Method E: Sulfo-cyanine 5 Labeling of the Acyloxymethyl Ketones (AOMK).

he boc-protected acyloxymethyl ketone (AOMK) (1 equiv.) was treated with a 1:1 mixture of TFA and CH₂Cl₂ (2 mL) and stirred at ambient temperature for 1 h. Volatile components were removed under a stream of nitrogen. To the resulting residue was added DMF (300 µL) and DIPEA (4 equiv.). Separately, Sulfo-Cy5 (1 equiv.) and PyClock (2 equiv.) were dissolved in DMF (300 µL) and DIPEA (4 equiv.) and stirred for 2 min before adding to the above solution. The reaction mixture was stirred at ambient temperature for 18 h excluding light.

### Example 2.1: Synthesis and Characterization Of Intermediate Chloromethyl Ketones

tert-butyl (S)-(1-chloro-2-oxoheptan-3-yl)carbamate (SJM-724-148)

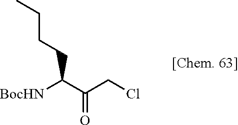 [SMILES: CCCC[C@H](NC(=O)OC(C)(C)C)C(=O)CCl]

The title compound was prepared via Method Cfrom tert-butyl (S)-(1-(dimethyl(oxo)-λ⁶-sulfanylidene)-2-oxoheptan-3-yl)carbamate (SJM-724-124) (40 mg, 0.13 mmol). The resulting crude product was purified by column chromatography (SiO₂, Petroleum Spirits : EtOAc, 9:1) to yield the product as a white solid (20 mg, 58%). ¹H NMR(401 MHz, CDCl₃) δ 5.02 (d, J = 6.4 Hz, 1H), 4.47 (dd, J = 12.6, 7.7 Hz, 1H), 4.26 (d, J = 4.8 Hz, 2H), 1.92 -1.75 (m, 1H), 1.60 - 1.47 (m, 1H), 1.43 (s, 9H), 1.39 - 1.25 (m, 4H), 0.90 (t, J = 7.1 Hz, 3H). ¹³CNMR(101 MHz, CDCl₃) δ 202.0 (C), 155.6 (C), 80.4 (C), 57.5 (CH), 46.8 (CH₂), 31.3 (CH₂), 28.4 (CH₃), 27.6 (CH₂), 22.5 (CH₂), 13.9 (CH₃). LC-MS (ESI⁺) m/z: 164.1.0 (M - Boc + H)⁺(100%).

tert-butyl (S)-(4-chloro-3-oxo-1-phenylbutan-2-yl)carbamate (SJM-724-152)

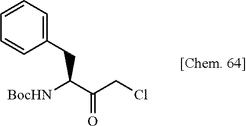 [SMILES: CC(C)(C)OC(=O)N[C@@H](Cc1ccccc1)C(=O)CCl]

The title compound was prepared via Method Cfrom tert-butyl (S)-(4-(dimethyl(oxo)-λ⁶-sulfanylidene)-3-oxo-1-phenylbutan-2-yl)carbamate (SJM-724-72) (50 mg, 0.15 mmol). The resulting crude product was purified by column chromatography (SiO₂, Petroleum Spirits : EtOAc, 9:1) to yield the product as a white solid (30 mg, 68%). HRMS (ESI⁺): Found: m/z 320.1018 (M + Na)⁺, C₁₅H₂₀ClNNaO₃⁺ requires m/z 320.1024. ¹H NMR(401 MHz, CDCl₃) δ 7.35 - 7.23 (m, 3H), 7.19 - 7.13 (m, 2H), 5.06 (d, J = 5.7 Hz, 1H), 4.66 (dd, J = 13.0, 6.0 Hz, 1H), 4.17 (d, J = 16.2 Hz, 1H), 3.98 (d, J = 16.2 Hz, 1H), 3.08 (dd, J = 13.8, 6.7 Hz, 1H), 2.99 (dd, J = 13.4, 7.1 Hz, 1H), 1.40 (s, 9H). ¹³CNMR(101 MHz, CDCl₃) δ 201.5 (C), 155.3 (C), 135.7 (C), 129.3 (CH), 129.1 (CH), 127.5 (CH), 80.6 (C), 58.6 (CH), 47.6 (CH₂), 37.8 (CH₂), 28.4 (CH₃). LC-MS (ESI⁺) m/z: 198.0 (M - Boc + H)⁺ (100%).

benzyl tert-butyl (7-chloro-6-oxoheptane-1,5-diyl)(S)-dicarbamate (SJM-724-52)

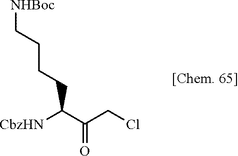 [SMILES: CC(C)(C)OC(=O)NCCCC[C@H](NC(=O)OCc1ccccc1)C(=O)CCl]

The title compound was prepared via Method Cfrom benzyl tert-butyl (7-(dimethyl(oxo)-λ⁶-sulfanylidene)-6-oxoheptane-1,5-diyl)(S)-dicarbamate (SJM-724-48) (130 mg, 0.29 mmol). The resulting crude product was purified by column chromatography (SiO₂, Petroleum Spirits : EtOAc, 3:1) to yield the product as a white solid (60 mg, 51%). HRMS (ESI⁺): Found: m/z 435.1664 (M + Na)⁺, C₂₀H₂₉ClN₂NaO₅⁺ requires m/z 435.1657. ¹H NMR(401 MHz, CDCl₃) δ 7.38 - 7.27 (m, 5H), 5.68 (d, J = 5.7 Hz, 1H), 5.09 (s, 2H), 4.62 (s, 1H), 4.59 - 4.47 (m, 1H), 4.26 (d, J = 2.1 Hz, 2H), 3.16 - 3.00 (m, 2H), 1.92 - 1.81 (m, 1H), 1.70 - 1.57 (m, 1H), 1.54 - 1.27 (m, 4H), 1.40 (s, 9H). ¹³CNMR(101 MHz, CDCl₃) δ 201.6 (C), 156.4 (C), 136.1 (C), 128.7 (CH), 128.4 (CH), 128.3 (CH), 79.4 (C), 67.3 (CH₂), 57.9 (CH), 46.6 (CH₂), 39.6 (CH₂), 30.8 (CH₂), 29.8 (CH₂), 28.5 (CH₃), 22.2 (CH₂). LC-MS (ESI⁺) m/z: 434.9.0 (M + Na)⁺ (100%).

### Example 2.2: Synthesis and Characterization of Acyloxymethyl Ketones (AOMK)

(S)-3-((tert-butoxycarbonyl)amino)-2-oxoheptyl 2,6-dimethylbenzoate (SJM-724-156)

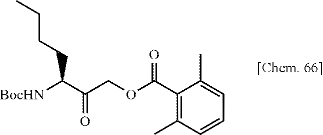 [SMILES: CCCC[C@H](NC(=O)OC(C)(C)C)C(=O)COC(=O)c1c(C)cccc1C]

The title compound was prepared via Method D from tert-butyl (S)-(1-chloro-2-oxoheptan-3-yl)carbamate (SJM-724-148) (20 mg, 76 µmol). The resulting crude product was purified by column chromatography (SiO₂, Petroleum Spirits : EtOAc, 95:5) to yield the product as a white solid (18 mg, 63%). HRMS (ESI⁺): Found: m/z 400.2097 (M + Na)⁺, C₂₁H₃₁NNaO₅⁺ requires m/z 400.2094. ¹H NMR(401 MHz, CDCl₃) δ 7.23 - 7.17 (m, 1H), 7.04 (d, J = 7.8 Hz, 2H), 5.15 - 4.96 (m, 3H), 4.40 (dd, J = 12.5, 7.7 Hz, 1H), 2.39 (s, 6H), 1.97 - 1.86 (m, 1H), 1.67 - 1.54 (m, 1H), 1.45 (s, 9H), 1.41 - 1.30 (m, 4H), 0.94 - 0.88 (m, 3H). ¹³CNMR(101 MHz, CDCl₃) δ 202.8 (C), 169.1 (C), 155.7 (C), 135.8 (C), 132.7 (C), 129.8 (CH), 127.8 (CH), 80.4 (C), 66.9 (CH₂), 57.0 (CH), 31.4 (CH₂), 28.4 (CH₃), 27.4 (CH₂), 22.5 (CH₂), 20.0 (CH₃), 14.0 (CH₃). LC-MS (ESI⁺) m/z: 399.9 (M + Na)⁺(100%).

(S)-3-((tert-butoxycarbonyl)amino)-2-oxo-4-phenylbutyl 2,6-dimethylbenzoate (SJM-724-172)

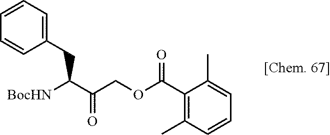 [SMILES: Cc1cccc(C)c1C(=O)OCC(=O)[C@H](Cc1ccccc1)NC(=O)OC(C)(C)C]

The title compound was prepared via Method D from tert-butyl (S)-(4-chloro-3-oxo-1-phenylbutan-2-yl)carbamate (SJM-724-152) (25 mg, 84 µmol). The resulting crude product was purified by column chromatography (SiO₂, Petroleum Spirits : EtOAc, 95:5) to yield the product as a white solid (27 mg, 78%). HRMS (ESI⁺): Found: m/z 434.1944 (M + Na)⁺, C₂₄H₂₉NNaO₅⁺ requires m/z 434.1938. ¹H NMR(401 MHz, CDCl₃) δ 7.35 - 7.18 (m, 6H), 7.05 (d, J = 7.6 Hz, 2H), 5.06 (d, J = 7.3 Hz, 1H), 4.94 (dd, J = 43.1, 17.0 Hz, 2H), 4.62 (dd, J = 14.0, 7.0 Hz, 1H), 3.18 (dd, J = 14.0, 6.4 Hz, 1H), 3.04 (dd, J = 14.0, 7.2 Hz, 1H), 2.39 (s, 6H), 1.42 (s, 9H). ¹³CNMR(101 MHz, CDCl₃) δ 202.2 (C), 169.1 (C), 155.4 (C), 135.9 (C), 135.8 (C), 132.7 (C), 129.8 (CH), 129.5 (CH), 128.9 (CH), 127.8 (CH), 127.3 (CH), 80.5 (C), 67.3 (CH₂), 58.1 (CH), 37.5 (CH₂), 28.4 (CH₃), 20.0 (CH₃). LC-MS (ESI⁺) m/z: 433.8 (M + Na)⁺(100%).

(S)-3-(((benzyloxy)carbonyl)amino)-7-((tert-butoxycarbonyl)amino)-2-oxoheptyl 2,6-dimethylbenzoate (SJM-724-176)

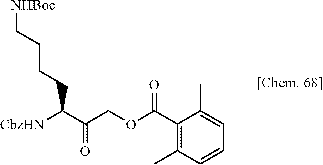 [SMILES: Cc1cccc(C)c1C(=O)OCC(=O)[C@H](CCCCNC(=O)OC(C)(C)C)NC(=O)OCc1ccccc1]

The title compound was prepared via Method D from benzyl tert-butyl (7-chloro-6-oxoheptane-1,5-diyl)(S)-dicarbamate (SJM-724-52) (50 mg, 121 µmol). The resulting crude product was purified by column chromatography (SiO₂, Petroleum Spirits : EtOAc, 3:1) to yield the product as a white solid (57 mg, 89%). HRMS (ESI⁺): Found: m/z 549.2571 (M + Na)⁺, C₂₉H₃₈N_{&shy;2}NaO₇⁺ requires m/z 549.2571. ¹HNMR(401 MHz, CDCl₃) δ 7.38 - 7.29 (m, 5H), 7.23 - 7.17 (m, 1H), 7.04 (d, J = 7.5 Hz, 2H), 5.66 (d, J = 5.9 Hz, 1H), 5.11 (s, 2H), 5.12 - 4.95 (m, 2H), 4.65 (s, 1H), 4.52 - 4.41 (m, 1H), 3.18 - 3.00 (m, 2H), 2.39 s, 6H), 2.04 - 1.89 (m, 1H), 1.78 - 1.64 (m, 1H), 1.58 - 1.35 (m, 4H), 1.41 (s, 9H). ¹³CNMR(101 MHz, CDCl₃) δ 202.3 (C), (C), 156.4 (C), 136.2 (C), 135.8 (C), 132.5 (C), 129.9 (CH), 128.7 (CH), 128.4 (CH), 128.3 CH), 127.8 (CH), 79.3 (C), 67.3 (CH₂), 66.8 (CH₂), 57.5 (CH), 39.7 (CH₂), 30.9 (CH₂), 29.8 (CH₂), 28.005 (CH₃), 22.0 (CH₂), 20.0 (CH₃). LC-MS (ESI⁺) m/z: 426.9 (M -Boc + H)⁺ (100%).

### Example 2.3: Synthesis and Characterization Of Acyloxymethyl Ketone (AOMK) Probes

sCy5-Nle-AOMK (SJM-724-160)

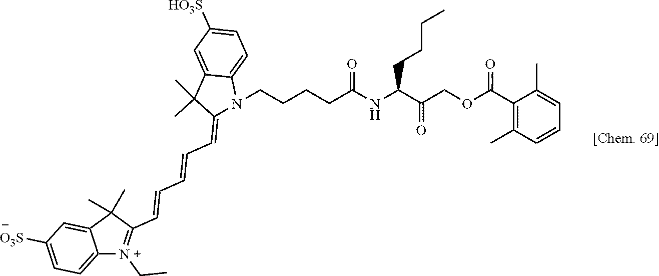

The title compound was prepared via Method E from (S)-3-((tert-butoxycarbonyl)amino)-2-oxoheptyl 2,6-dimethylbenzoate (SJM-724-156) (8 mg, 21 µmol). The resulting crude product was purified by RP-HPLCand lyophilised to yield the product as a blue solid (13.9 mg, 72%). HRMS (ESI⁺): Found: m/z 916.3869 (M + H)⁺, C₄₉H₆₂N₃O₁₀S₂⁺ requires m/z 916.3871.

sCy5-Phe-AOMK (SJM-724-180)

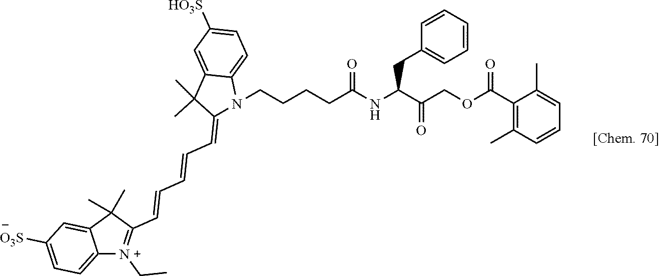

The title compound was prepared via Method E from (S)-3-((tert-butoxycarbonyl)amino)-2-oxo-4-phenylbutyl 2,6-dimethylbenzoate (SJM-724-172) (5 mg, 12 µmol). The resulting crude product was purified by RP-HPLCand lyophilised to yield the product as a blue solid (6.5 mg, 56%). HRMS (ESI⁺): Found: m/z 950.3718 (M + H)⁺, C₅₂H₆₀N₃O₁₀S₂⁺ requires m/z 950.3715.

Cbz-Lys(sCy5)-AOMK (SJM-724-184)

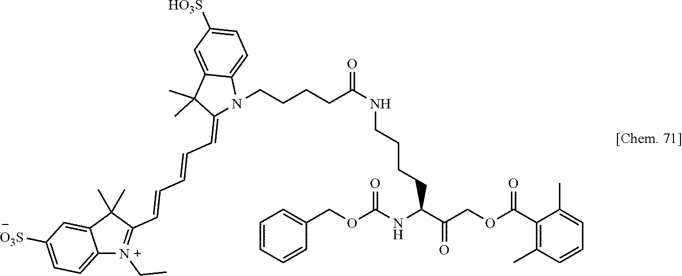

The title compound was prepared via Method E from (S)-3-(((benzyloxy)carbonyl)amino)-7-((tert-butoxycarbonyl)amino)-2-oxoheptyl 2,6-dimethylbenzoate (SJM-724-176) (5 mg, 9 µmol). The resulting crude product was purified by RP-HPLCand lyophilised to yield the product as a blue solid (6.5 mg, 64%). HRMS (ESI⁺): Found: m/z 1065.4351 (M + H)⁺, C₅₇H₆₉N₄O₁₂S₂⁺ requires m/z 1065.4348.

### Testing of Probes

### General Information

### Methods

TABLE 1

Key Resource TABLE

| REAGENT or RESOURCE | SOURCE | IDENTIFIER |
|---|---|---|
| Antibodies | | |
| Mouse cathepsin X/Z/P antibody (polyclonal goat IgG) | R&D | AF1033 |
| Alexa Fluor® 594 AffiniPure Donkey Anti-Goat IgG (H+L) | Jackson ImmunoResearch | 705-585-003 |
| Chemicals, Peptides, and Recombinant Proteins | | |
| Sulfoxonium Ylide Probes | Synthesized according to Section I above | N/A |
| BMV109 | Synthesized in house | (Verdoes et al., 2013) |
| JPM-OEt | Drug Synthesis & Chemistry Branch, Division of Cancer Treatment and Diagnosis, National Cancer Institute | (Meara and Rich, 1996) |
| MDV-590 | Medivir Pharmaceuticals, Sweden | N/A |
| Critical Commercial Assays | | |
| BCA Assay | Pierce | PIE23223/PIE23224 |
| Protein A/G PLUS-Agarose | Santa Cruz | SC-2003 |
| Experimental Models: Cell Lines | | |
| RAW264.7 | ATCC | ATCCTIB-71 |
| MDA-MB-231^{HM} | Dr Zhou Ou, Fudan University, Shanghai Cancer Center | (Chang et al., 2007) |
| Experimental Models: Organisms/Strains | | |
| Cathepsin X/Z-deficient mice | Yates Lab, University of Calgary | (Sevenich et al., 2010) |
| Wildtype C57BL/6J mice | Bred at Monash Animal Resesarch Platform, originally from The Jackson Laboratory | 000664 |

### Experimental Model and Subject Details

### Cell Culture

RAW264.7 or MDA-MB-231 ^{HM} cells were cultured in DMEM containing 10% fetal bovine serum and 1% antibiotic/antimycotic. RAW264.7 cells were passaged by scraping with a rubber policeman, while MDA-MB-231 ^{HM} cells were lifted with 0.02% EDTA in phosphate-buffered saline (PBS).

### Animals

All experiments involving animals were approved by the Monash University Animal Ethics Committee. Male C57BL/6J mice were obtained from the Monash Animal Research Platform and used at 8-10 weeks of age. Snap-frozen spleens from wildtype and cathepsin X knockout mice, as described in (Sevenich et al., 2010), were obtained from the University of Calgary in accordance with the University of Calgary Animal Care and Use Committee.

### Method Details

### Cell Lysate Labeling and SDS-PAGE Analysis

ells were harvested by scraping, washed once with PBS, and resuspended in lysis buffer containing 50 mM citrate [pH 5.5], 0.5% CHAPS, 0.1% Triton X-100, and 4 mM DTT. Cells were incubated on ice for at least 10 minutes with intermittent vortexing followed by centrifugation (21 g at 4° C. or 5 minutes). Cleared supernatants were then transferred to a fresh tube and protein concentration was determined by BCA. Total protein (50 µg) was aliquoted into tubes in a final volume of 20 µl lysis buffer. Where indicated, JPM-OEt or MDV-590 were added from a 100x DMSO stock and incubated at 37° C. or 20 minutes prior to probe addition. The indicated concentration of probe was added from a 100x DMSO stock. Labeling was carried out at 37° C. or 20 minutes (unless otherwise indicated), and the reactions were quenched by the addition of 5x sample buffer (200 mM Tris-Cl [pH 6.8], 8% SDS, 0.04% bromophenol blue, 5% b-mercaptoethanol, and 40% glycerol). Samples were then boiled for five minutes and proteins were resolved on a 15% SDS-PAGE gel. The gels were scanned on a Typhoon 5 flatbed laser scanner at 633/670 nm excitation/emission to detect sCy5 fluorescence.

### Live Cell Labeling

AW cells or MDA-MB-231 ^{HM} cells were plated in 12-well plates. Where indicated, MDV-590 or vehicle was added at 10 µM from a 10 mM DMSO stock for overnight incubation. When the cell density reached 80%, the indicated probes were added at the indicated concentrations from a 1000x DMSO stock and allowed to incubate for the indicated time. Media was then removed and replaced with PBS. The cells were then scraped and transferred to tubes, and lysis and SDS-PAGE analysis were carried out as above, except skipping the probe addition step.

### Tissue Analysis

issues were harvested from healthy mice and snap frozen. At the time of analysis, lysis buffer was added at 10x volume:weight, and tissues were sonicated on ice. Cleared lysates were labeled with the indicated probe and analyzed as above.

For in vivo labeled tissues, mice were first injected intravenously via the tail vein with sCy5-Nle-SY, BMV109, or sCy5-Nle-AOMK (50 nmol in 100 µl 10% DMSO/PBS or vehicle control). Tissues were harvested and analyzed as above except without further probe addition.

### Immunoprecipitation Assay 0.5

robe-labeled lysate from above (in sample buffer) was divided into input or pulldown (~50 µg total protein each). The input sample was stored at -20° C. The pulldown sample was diluted in 500 µl IP buffer (PBS [pH 7.4],% NP-40, 1 mM EDTA). Goat anti-cathepsin X antibody (10 µl) was added along with 40 µl slurry of pre-washed Protein A/G agarose beads. Samples were rotated overnight at 4° C. Beads were then washed four times with IP buffer followed by a final wash in 0.9% NaCl. Beads were then resuspended in 2x sample buffer and boiled. The pulldown supernatants, alongside the input samples, were analysed by fluorescent SDS-PAGE as above.

### Confocal Microscopy

Kidney tissues from mice that received sCy5-Nle-SY (or vehicle control) above were fixed overnight in 4% paraformaldehyde in PBS followed by overnight cryoprotection in 30% sucrose. Tissues were embedded in OCT, frozen on dry ice, and sectioned at 10 µm. Immunostaining for cathepsin X was carried out according to standard protocols. In brief, sections were air dried, fixed in cold acetone for 10 minutes, air dried again, and then rehydrated in PBS. Sections were blocked in PBS containing 3% normal horse serum with 0.1% Triton X-100. Goat anti-cathepsin X was added at 1:100 in blocking buffer overnight at 4° C. Sections were then washed, and secondary antibody, donkey anti-goat-AlexaFluor594 was added at 1:500 for 1 hour at room temperature. Sections were stained with DAPI for 5 minutes, washed, and mounted with ProLong Diamond. Staining was analyzed using a Leica SP8 inverted confocal microscope.

### Example 3 - Labeling With sCy5-Val-SY in Cell Lysates

In Example 3, the sulfoxonium ylide probe sCy5-Val-SY was incubated with protein lysates prepared from RAW264.7 cells, an immortalized mouse macrophage line that contains high levels of active cysteine cathepsins (Verdoes et al., 2013). Cells were lysed in citrate buffer (pH 5.5) to provide optimal conditions for cathepsin activation, and the probe was added
at 1 µM for 20 minutes
at various concentrations (0 µM, 0.01 µM, 0.05 µM, 0.1 µM, 0.5 µM or 1 µM) for 20 minutes; or
at 1 µM for varying times (0, 1, 2, 5, 10, 20, and 30 minutes).

Then the lysates were resolved by SDS-PAGE and the gel scanned for sCy5 fluorescence using a flatbed laser scanner. Exclusive, concentration- and time-dependent labeling of a ~35-KDa protease was observed (FIG. 2A, FIG. 2A-1, and FIG. 2A-2). This labeling was prevented by pretreatment of the lysates with JPM-OEt, a pan-cysteine cathepsin inhibitor, confirming that this protease was a member of the cysteine cathepsin family (FIG. 2A). In contrast, MDV-590 - a specific inhibitor for cathepsin S - did not compete for sCy5-Val-SY binding.

The labeling profile of sCy5-Val-SY was compared to that of BMV109, the pan-cathepsin probe, by repeating the above experiment using BMV109 as the probe added at 1 µM for 20 minutes. It was found that the sCy5-Val-SY-labeled protease was the same molecular weight as BMV109-labeled cathepsin X (Verdoes et al., 2013). It was confirmed that this protease was indeed cathepsin X by immunoprecipitating sCy5-Val-SY-labeled lysates (sCy5-Val-SY added at 1 µM for 20 minutes) with a cathepsin X-specific antibody (FIG. 2B).

### Example 4 - Labeling With sCy5-Val-SY in Tissue Lysates

In Example 4, the ability of sCy5-Val-SY to label cathepsin X in mouse splenic lysates (from wildtype mice or cathepsin X-deficient mice) was tested (probe addition at 1 µM for 20 minutes). As observed in macrophage lysates, the probe exhibited exclusive reactivity with cathepsin X in splenic lysates from wildtype mice, and this labeling was absent in lysates prepared from spleens of cathepsin X-deficient mice (FIG. 2C). By comparison, BMV109 (added at 1 µM for 20 minutes) strongly labeled cathepsin B and to a lesser extent, cathepsin S and L (FIG. 2C).

The results of Examples 3 and 4 thus demonstrate exclusive specificity of sCy5-Val-SY in cell and tissue lysates.

### Example 5 - Labeling With sCy5-Val-SY in Living Cells

In Example 5, the permeability of the sCy5-Val-SY probe and its specificity profile in living RAW264.7 cells were assessed and compared to the BMV109 probe. After incubating the probe (at 1 µM) with live cells for increasing lengths of time (0, 1, 5, 10, 15, 25, 35, 45, 55, 65, 90 and 120 minutes) or with increasing probe concentrations (0 µM, 0.01 µM, 0.05 µM, 0.1 µM, 0.5 µM and 1 µM each for 2 hours), the lysates were analyzed by in-gel fluorescence as above. Here, time- and concentration-dependent labeling of two proteases was observed (FIG. 2D, FIG. 2D-1). The latter were identified as cathepsin X and S by immunoprecipitation (FIG. 2E) and competition with MDV-590 (FIG. 2F), respectively.

It was surprising to see cathepsin S labeling in live cells, given its lack of binding to sCy5-Val-SY in cell lysates, where high levels of cathepsin S activity had been confirmed with the BMV109 probe. This suggests that the reactivity of cathepsin S with the sulfoxonium ylide probe is dependent on the labeling conditions. An attempt was made to explore this by lysing the cells in various buffers that might mimic the endosomal environment of cathepsin S, but the labeling of cathepsin S in lysates could not be improved (results not shown).

Nonetheless, the sulfoxonium ylide probe exhibited clear labeling of cathepsin X in lysates and live cells with considerably improved selectivity compared to BMV109 (FIGS. 2D,F). To our knowledge, the sulfoxonium ylide probe is the first covalent ABP for cathepsin X that does not also bind to cathepsin B or L. As observed in FIG. 2A, FIG. 2C, FIG. 2D and FIG. 2F, it is difficult to distinguish cathepsin X labeling from cathepsin B with BMV109 due to the similarity in size of the two proteases. However, sCy5-Val-SY allows for clear delineation of cathepsin X activity.

### Example 6 - Labeling With Sulfoxonium Ylide Probes in Cell Lysates

In Example 6, a small library of sulfoxonium ylide probes with varying amino acids in the P1 position as prepared in accordance with Example 1 was tested in RAW264.7 lysates, and compared to BMV109. Each probe was added with increasing probe concentrations of 0.01 µM, 0.05 µM, 0.1 µM, 0.5 µM, 1 µM and 5 µM for 20 minutes).

TABLE 2

Tested sulfoxonium ylide probes:
Cbz-Lys(sCv5)-SY
sCY5-Val-SY
sCY5-lle-SY
sCy5-Leu-SY
sCy5-Nle-SY
sCy5-Phe-SY
sCy5-Phe-Val-SY

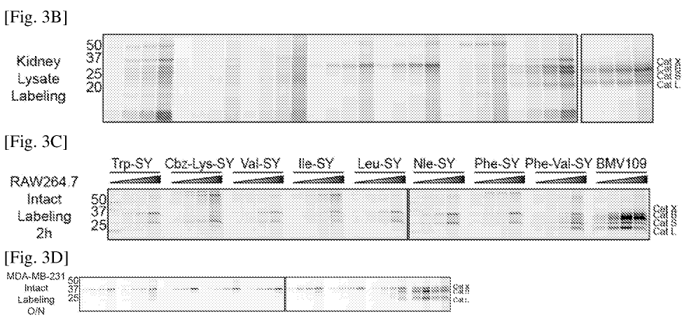

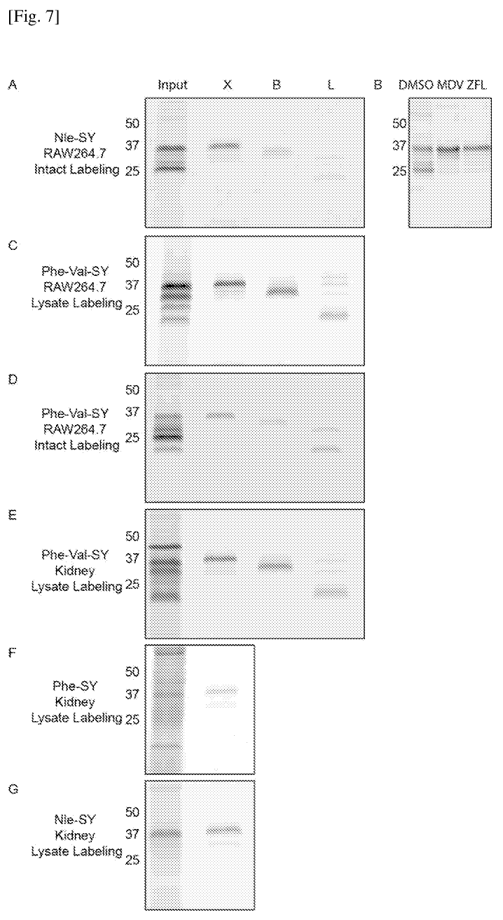

In the RAW264.7 lysates, probes bearing Ile, Leu, Nle, and Phe all showed similar specificity for cathepsin X as sCy5-Val-SY, with sCy5-Nle-SY being the most potent (FIG. 3A). The probe bearing Cbz-Lys, in which the sCy5 was attached via the lysine side chain, exhibited a loss of specificity, favoring cathepsin S over X and B. sCy5-Phe-Val-SY, in which a P2 Phe residue was incorporated, also exhibited a loss of specificity (FIG. 3A, FIG. 7). The labeling profile of this probe was similar to BMV109, though it showed improved potencies for cathepsin X and S compared to BMV109.

### Example 7 - Labeling With Sulfoxonium Ylide Probes in Kidney Lysates

In Example 7, sulfoxonium ylide probes as prepared in accordance with Example 1 were tested in kidney lysates and the results compared to BMV109. The sulfoxonium ylide probes (and BMV109) were each added with increasing probe concentrations of 0.1 µM, 0.5 µM, 1 µM and 5 µM for 20 minutes.

TABLE 3

Tested sulfoxonium ylide probes:
Cbz-Lys(sCY5)-SY
sCy5- Val-SY
sCy5-lle-SY
sCy5-Leu-SY
sCy5-Nle-SY
sCy5-Phe-SY
sCy5-Phe-Val-SY

In the kidney lysates, Leu and Nle conferred the most potency and specificity for cathepsin X, with Cbz-Lys, Phe, and Phe-Val yielding broader reactivity, and Val and Ile exhibiting weaker labeling (FIG. 3B, FIG. 7).

### Example 8 - Labeling With Sulfoxonium Ylide Probes in Live RAW264.7 Cells

In Example 8, sulfoxonium ylide probes as prepared in accordance with Example 1 were applied to live RAW264.7 cells for two hours, in order to examine the potency and permeability of the sulfoxonium ylide probe series in living cells, and the results compared to BMV109.

The sulfoxonium ylide probes (and BMV109) were each added with increasing probe concentrations of 0.1 µM, 0.5 µM, 1 µM and 5 µM for 2 hours.

TABLE 4

Tested sulfoxonium ylide probes:
sCy5-Trp-SY
Cbz-Lys(sCy5)-SY
sCy5-Val-SY
sCy5-lle-SY
sCy5-Leu-SY
sCy5-Nle-SY
sCy5-Phe-SY
sCy5-Phe-Val-SY

Probes bearing Trp, Val, Ile, Leu, Nle, and Phe labeled cathepsin X and S to similar extents and with similar potency, while Cbz-Lys exhibited a preference for cathepsin S and Phe-Val labeled cathepsin B and L in addition to cathepsin X and S (FIG. 3C, FIG. 7).

### Example 9 - Labeling With Sulfoxonium Ylide Probes in Live MDA-MB-231 ^{HM} Cells

In Example 9, the specificity of the sulfoxonium ylide probes for cathepsin X in a human breast cancer line known to express very low levels of cathepsin S, MDA-MB-231 ^{HM} (Chang et al., 2007) was tested, and the results compared to BMV109. The MDA-MB-231 ^{HM} cells also allowed to test whether the sulfoxonium ylide probes could bind to human cathepsin X (in addition to mouse cathepsin X shown previously).

The sulfoxonium ylide probes (and BMV109) were each added with increasing probe concentrations of 0.1 µM, 0.5 µM, 1 µM and 5 µM).

TABLE 5

Tested sulfoxonium ylide probes:
sCy5-Trp-SY
Cbz-Lys(sCy5)-SY
sCy5-Val-SY
sCy5-lle-SY
sCy5-Leu-SY
sCy5-Nle-SY
sCy5-Phe-SY
sCy5-Phe-Val-SY

When the probes were incubated with MDA-MB-231 cells for shorter time periods, very little labeling of cathepsin X was observed (results not shown). However, clear labeling was observed after overnight incubation (FIG. 3D). This likely reflects differences in the rates of endocytosis between macrophages and tumor cells and suggests that the probes may be taken up directly into the endolysosmal pathway rather than by diffusion through membranes. The sulfoxonium ylide probe series generally showed specific labeling of cathepsin X in these cells, with minimal cross-reactivity occurring only at 5 µM. sCy5-Lys-SY and especially sCy5-Phe-Val-SY exhibited the most cross-reactivity with cathepsin B and L.

### Example 10 - in Vivo Characterization Of sCy5-Nle-SY

Taking into consideration all of the data from cell and tissue lysates and live mouse macrophages and human cancer cells, the sulfoxonium ylide probe sCy5-Nle-SY emerged as the probe showing the highest potency and selectivity for cathepsin X. Thus, this probe was elected for in vivo studies.

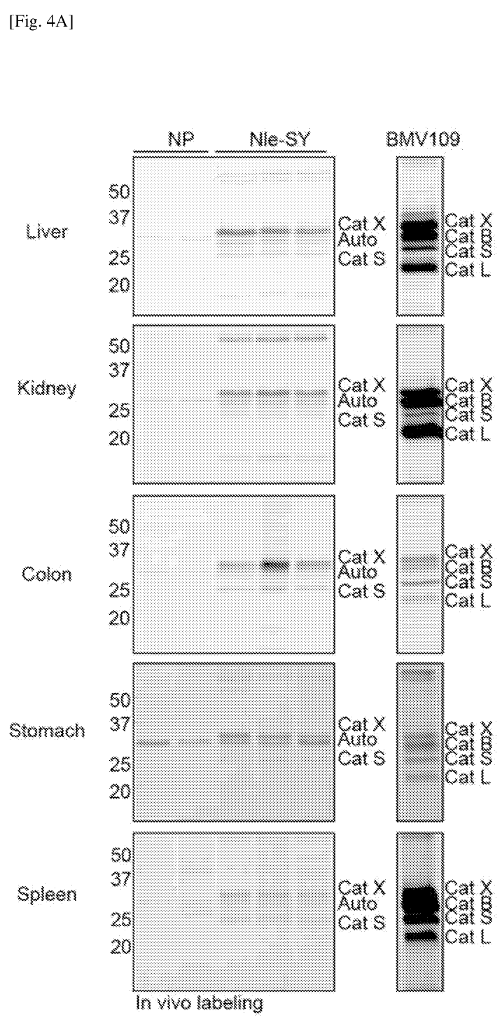

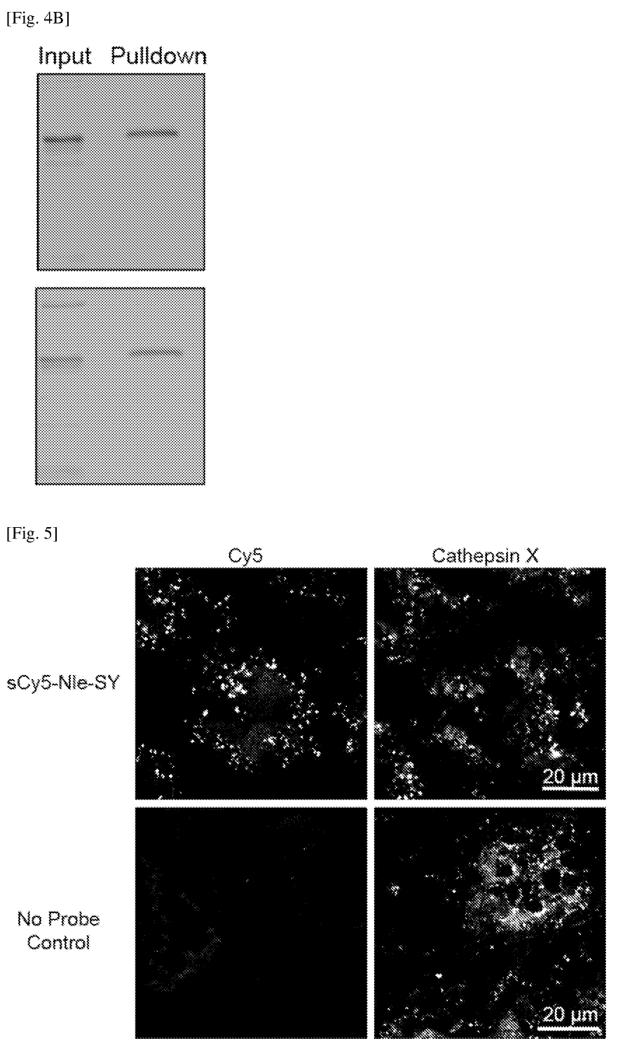

In Example 10, the probe sCy5-Nle-SY, vehicle control/no probe (NP) or BMV109 were injected into mice intravenously. After two hours of circulation, tissues were harvested, lysed, and analyzed for probe labeling by fluorescent SDS-PAGE. Labeling of cathepsin X was observed in liver, kidney, colon, stomach, and spleen (FIG. 4A), and this was confirmed by immunoprecipitation with a cathepsin X antibody (FIG. 4B). While some labeling of cathepsin S was also observed, the overall specificity profile was clearly improved compared to BMV109, which also strongly labels cathepsin B and L.

Additionally, after in vivo probe administration, kidney cryosections from sCy5-Nle-SY-injected mice or no-probe control were prepared and analyzed by confocal microscopy for sCy5 fluorescence (red) or cathepsin X immunoreactivity (green) along with DAPI (blue) to visualize nuclei. The results are presented in FIG. 5. Strong punctate sCy5 fluorescence was observed reminiscent of endolysomal staining, and this signal largely overlapped with immunoreactive cathepsin X (FIG. 5). Thus, sCy5-Nle-SY could be used to distinguish active cathepsin X relative to total cathepsin X in tissues after in vivo administration.

### Example 11 - Characterization Of AOMK And Sulfoxonium Ylide Probes

In Example 11, the reactivity of the new AOMK probes prepared according to Example 2 was compared with the corresponding sulfoxonium ylide probes prepared according to Example 1, using a labeling experiment in living RAW264.7 cells.

TABLE 6

| | |
|---|---|
| Tested AOMK probes: | Tested sulfoxonium ylide probes: |
| Cbz-Lys(sCy5)-AOMK | Cbz-Lys(sCy5)-SY |
| sCy5-Nle-AOMK | sCy5-Nle-SY |
| sCy5-Phe-AOMK | sCy5-Phe-SY |

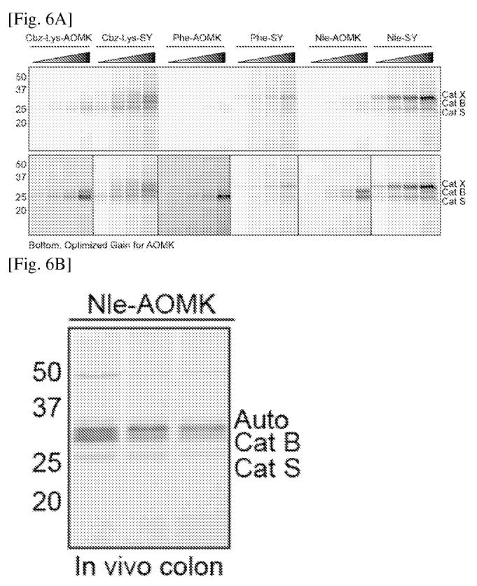

The probes were added at increasing concentrations (0.1, 0.5, 1, 5 µM) for two hours and labeling was analyzed by in-gel fluorescence. The results are shown in FIG. 6A.

The AOMK probes were much less potent than the ylide probes, suggesting reduced reactivity. These probes labeled cathepsin B and S, but not X (FIG. 6A), which is in line with previous data demonstrating limited reactivity of cathepsin X with the AOMK warhead (Paulick and Bogyo, 2011).

### Example 12 - in Vivo Characterization of sCy5-Nle-AOMK

In Example 12, the AOMK probe sCy5-Nle-AOMK was tested in vivo and its labeling in tissues was analyzed. The probe was injected into mice intravenously. After two hours of circulation, tissues were harvested, lysed, and analyzed for probe labeling by fluorescent SDS-PAGE. Only weak labeling of cathepsin B and S was observed in the colon, but not in other tissues examined (FIG. 6B).

### Example 13 - Labeling with sCy5-Nle-SY in Human Tissues

To investigate the ability of sCy5-,Nle-SY to label cathepsin X in human tissues, biopsies that were obtained from patients with oral squamous cell carcinoma were used. Normal oral mucosal tissues that were biopsied from the contralateral side of the tongues of the same patients were used as controls.

The human oral squamous cell carcinomas or patient-matched normal oral mucosa were biopsied according to protocols approved by the Institutional Review Board at the New York University Oral Cancer Centre. Biopsies were immediately snap-frozen and stored at -80° C. Tissues were lysed by sonication in citrate buffer and supernatants were cleared by centrifugation. Total protein concentration was assessed by BCA assay and 80 µg were labelled with Cy5-Nle-SY: The lysates prepared from the oral squamous cell carcinoma tissue or patient-matched normal oral mucosa were incubated with sCy5-Nle-SY (°C5 µM for 20 minutes) and labeling was analyzed by in-gel fluorescence (fluorescent SDS-PAGE). Samples were also immunoblotted to determine total cathepsin X expression (n=10). Additionally, the sCy5-Nle-SY-labeled cancer lysates were immunoprecipitated with a cathepsin X-specific antibody to verify the identity of the labeled protein.

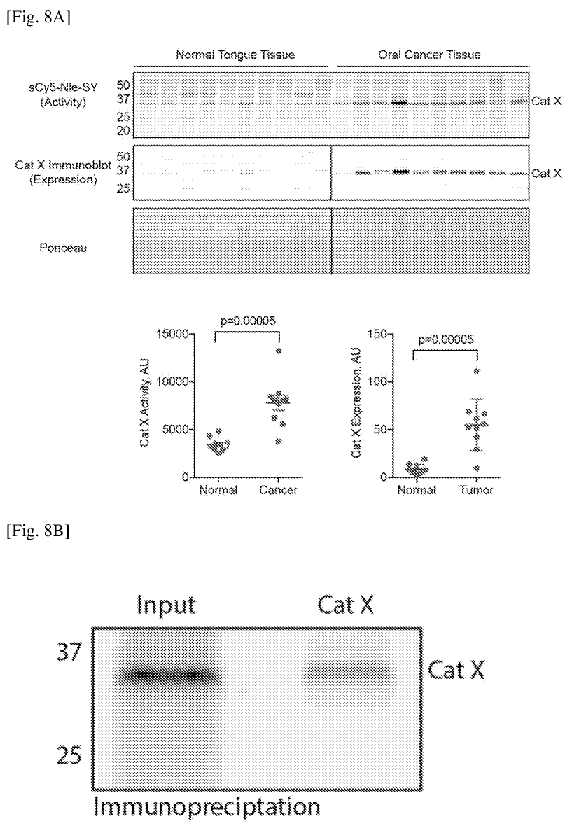

The results are shown in FIGS. 8A and 8B. In all of the cancer samples examined, clear and selective labeling of cathepsin X was observed, as confirmed by immunoprecipitation with a cathepsin X-specific antibody. Cathepsin X activity was significantly increased in cancer biopsies compared to normal tongue tissue, and this was corroborated by an increase in total cathepsin X, as measured by immunoblot. These data indicate that sCy5-Nle-SY can successfully detect cathepsin X activation in human tissues and reveal, for the first time, that cathepsin X is significantly upregulated in oral squamous cell carcinoma.

In summary, the results of the experiments carried out demonstrate that the tested new dimethyl sulfoxonium ylide warhead exhibits unique selectivity towards cysteine cathepsin proteases in cell lysates, live cells, in vivo (such as in mice) and in tissue lysates (mouse and human). The best probe of the tested sulfoxonium ylides, sCy5-Nle-SY is the most potent and selective probe for cathepsin X to date, showing exclusive specificity in cell lysates and cells that express low levels of cathepsin S. While this probe does cross-react with cathepsin S in live macrophages and in vivo, it does not label cathepsin B or L, which is a clear improvement over other probes targeting cathepsin X (BMV109, MGP140). The use of sCy5-Nle-SY allows for clear measurement of the activity of the cathepsin X by SDS-PAGE, whereas this was difficult with previous probes due to confounding levels of cathepsin B labeling. Furthermore, it was established that the sulfoxonium ylide warhead is stable enough for in vivo detection of cathepsin X and that the sCy5-Nle-SY signal was bright enough to detect by confocal microscopy.

### Example 14 - Boc-Val-SY as a Cathepsin X Inhibitor

To test the ability of sulfoxonium ylide compounds to act as cathepsin inhibitors, an unlabeled, boc-protected version of Val-SY, i.e. tert-butyl (S)-(1-(dimethyl(oxo)-λ ⁶-sulfanylidene)-4-methyl-2-oxopentan-3-yl)carbamate (SJM-724-24) was used. Live RAW264.7 cells were pre-treated with Boc-Val-SY (0, 10 or 100 µM) overnight. Residual cathepsin X activity was measured by incubating the live cells with sCy5-Val-SY for two hours. Cells were lysed and labeled proteins were detected by in-gel fluorescence.

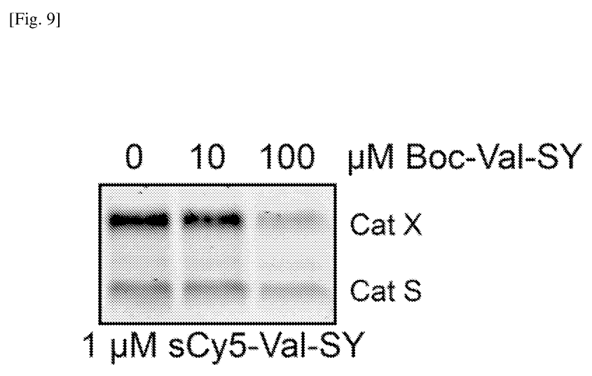

The results are shown in FIG. 9. Compared to controls, cells treated with 100 µM Boc-Val-SY exhibited a 71% and 51% reduction in cathepsin X and cathepsin S, respectively. These data demonstrate the ability of Boc-Val-SY to compete for sCy5-Val-SY labeling and thus act as a cathepsin inhibitor.

The present examples, methods, procedures, specific compounds and molecules are meant to exemplify and illustrate the invention and should in no way be seen as limiting the scope of the invention, which is defined by the literal and equivalent scope of the appended claims. Any patents or publications mentioned in this specification are indicative of levels of those skilled in the art to which the patent pertains and are intended to convey details of the invention which may not be explicitly set out but would be understood by workers in the field. Such patens or publications are hereby incorporated by reference to the same extent as if each was specifically and individually incorporated by reference and for the purpose of describing and enabling the method or material referred to.

### References

Akkari, L., Gocheva, V., Kester, J.C., Hunter, K.E., Quick, M.L., Sevenich, L., Wang, H.-W., Peters, C., Tang, L.H., Klimstra, D.S., Reinheckel, T., and Joyce, J.A. (2014). Distinct functions of macrophage-derived and cancer cell-derived cathepsin Z combine to promote tumor malignancy via interactions with the extracellular matrix. Genes Dev. 28, 2134-2150. Allan, E.R.O., Campden, R.I., Ewanchuk, B.W., Tailor, P., Bake, D.R., McKenna, N.T., Greene, C.J., Warren, A.L., Reinheckel, T., and Yates, R.M. (2017). A role for cathepsin Z in neuroinflammation provides mechanistic support for an epigenetic risk factor in multiple sclerosis. J Neuroinflammation 14, 1-11.

Bernhardt, A., Kuester, D., Roessner, A., Reinheckel, T., and Krueger, S. (2010). Cathepsin X-deficient Gastric Epithelial Cells in Co-culture with Macrophages: Characterization of cytokine response and migration capability after Helicobacter pylori infection. J. Biol. Chem. 285, 33691-33700.

Chang, X.-Z., Li, D.-Q., Hou, Y.-F., Wu, J., Lu, J.-S., Di, G.-H., Jin, W., Ou, Z.-L., Shen, Z.-Z., and Shao, Z.-M. (2007). Identification of the functional role of AF1Q in the progression of breast cancer. Breast Cancer Res. Treat. 111, 65-78. Duivenvoorden, H.M., Rautela, J., Edgington-Mitchell, L.E., Spurling, A., Greening, D.W., Nowell, C.J., Molloy, T.J., Robbins, E., Brockwell, N.K., Lee, C.S., Chen, M., Holliday, A., Selinger, C.I., Hu, M., Britt, K.L., Stroud, D.A., Bogyo, M., Moller, A., Polyak, K., Sloane, B.F., O’Toole, S.A., and Parker, B.S. (2017). Myoepithelial cell-specific expression of stefin A as a suppressor of early breast cancer invasion. J. Pathol. 243, 496-509.

Edgington, L.E., Berger, A.B., Blum, G., Albrow, V.E., Paulick, M.G., Lineberry, N., and Bogyo, M. (2009). Noninvasive optical imaging of apoptosis by caspase-targeted activity-based probes. Nat. Med. 15, 967-973.

Edgington, L.E., and Bogyo, M. (2013). In vivo imaging and biochemical characterization of protease function using fluorescent activity-based probes. Curr. Protoc. Chem. Biol. 5, 25-44.

Edgington, L.E., van Raam, B.J., Verdoes, M., Wierschem, C., Salvesen, G.S., and Bogyo, M. (2012). An Optimized Activity-Based Probe for the Study of Caspase-6 Activation. Chem. Biol. 19, 340-352.

Edgington, L.E., Verdoes, M., and Bogyo, M. (2011). Functional imaging of proteases: recent advances in the design and application of substrate-based and activity-based probes. Curr. Op. Chem. Biol. 15, 798-805.

Edgington, L.E., Verdoes, M., Ortega, A., Withana, N.P., Lee, J., Syed, S., Bachmann, M.H., Blum, G., and Bogyo, M. (2013). Functional Imaging of Legumain in Cancer Using a New Quenched Activity-Based Probe. J. Am. Chem. Soc. 135, 174-182.

Edgington-Mitchell, L.E., Rautela, J., Duivenvoorden, H.M., Jayatilleke, K.M., van der Linden, W.A., Verdoes, M., Bogyo, M., and Parker, B.S. (2015). Cysteine cathepsin activity suppresses osteoclastogenesis of myeloid-derived suppressor cells in breast cancer. Oncotarget 6, 27008-27022.

Hafner, A., Glavan, G., Obermajer, N., Zivin, M., Schliebs, R, and Kos, J. (2013). Neuroprotective role of γ-enolase in microglia in a mouse model of Alzheimer’s disease is regulated by cathepsin X. Aging Cell 12, 604-614.

Huynh, J.L., Garg, P., Thin, T.H., Yoo, S., Dutta, R., Trapp, B.D., Haroutunian, V., Zhu, J., Donovan, M.J., Sharp, A.J., and Casaccia, P. (2013). Epigenome-wide differences in pathology-free regions of multiple sclerosis-affected brains. Nat. Neurosci. 17, 121-130. Krueger, S., Kalinski, T., Hundertmark, T., Wex, T., K&uuml;ster, D., Peitz, U., Ebert, M., Nagler, D.K., Kellner, U., Malfertheiner, P., Naumann, M., R&ouml;cken, C., and Roessner, A. (2005). Up-regulation of cathepsin X inHelicobacter pylori gastritis and gastric cancer. J. Pathol. 207, 32-42.

Leichsenring, A., B&auml;cker, I., Wendt, W., Andriske, M., Schmitz, B., Stichel, C.G., and L&uuml;bbert, H. (2008). Differential expression of Cathepsin S and X in the spinal cord of a rat neuropathic pain model. BMCNeurosci. 9, 80-13.

Meara, J.P., and Rich, D.H. (1996). Mechanistic studies on the inactivation of papain by epoxysuccinyl inhibitors. J. Med. Chem. 39, 3357-3366.

Nagler, D.K., Kraus, S., Feierler, J., Mentele, R., Lottspeich, F., Jochum, M., and Faussner, A. (2010). A cysteine-type carboxypeptidase, cathepsin X, generates peptide receptor agonists. Int. Immunopharmacol. 10, 134-139.

Nagler, D.K., Kruger, S., Kellner, A., Ziomek, E., Menard, R., Buhtz, P., Krams, M., Roessner, A., and Kellner, U. (2004). Up-regulation of cathepsin X in prostate cancer and prostatic intraepithelial neoplasia. Prostate 60, 109-119.

Nagler, D.K., Zhang, R., Tam, W., Sulea, T., Purisima, E.O., and Menard, R. (1999). Human Cathepsin X: A Cysteine Protease with Unique Carboxypeptidase Activity. Biochem. 38, 12648-12654.

Obermajer, N., Doljak, B., Jamnik, P., Fonovic, U.P., and Kos, J. (2009). Cathepsin X cleaves the C-terminal dipeptide of alpha- and gamma-enolase and impairs survival and neuritogenesis of neuronal cells. Int. J. Biochem. Cell Biol. 41, 1685-1696.

Obermajer, N., Svajger, U., Bogyo, M., Jeras, M., and Kos, J. (2008). Maturation of dendritic cells depends on proteolytic cleavage by cathepsin X. J. Leukocyte Biol. 84, 1306-1315.

Oresic Bender, K., Ofori, L., van der Linden, W.A., Mock, E.D., Datta, G.K., Chowdhury, S., Li, H., Segal, E., Sanchez Lopez, M., Ellman, J.A., Figdor, C.G., Bogyo, M., and Verdoes, M. (2015). Design of a Highly Selective Quenched Activity-Based Probe and Its Application in Dual Color Imaging Studies of Cathepsin S Activity Localization. J. Am. Chem. Soc. 137, 4771-4777.

Orlowski, G.M., Colbert, J.D., Sharma, S., Bogyo, M., Robertson, S.A., and Rock, K.L. (2015). Multiple Cathepsins Promote Pro-IL-1β Synthesis and NLRP3-Mediated IL-1β Activation. J. Immunol. 195, 1685-1697.

Paulick, M.G., and Bogyo, M. (2011). Development of Activity-Based Probes for Cathepsin X. ACS Chem. Biol. 6, 563-572.

Pecar Fonovic, U., and Kos, J. (2015). Cathepsin X Cleaves Profilin 1 C-Terminal Tyr139 and Influences Clathrin-Mediated Endocytosis. PLoS ONE 10, e0137217-13. Sanman, L.E., and Bogyo, M. (2014). Activity-Based Profiling of Proteases. Annu. Rev. Biochem. 83, 249-273.

Sevenich, L., Schurigt, U., Sachse, K., Gajda, M., Werner, F., Muller, S., Vasiljeva, O., Schwinde, A., Klemm, N., Deussing, J., Peters, C., and Reinheckel, T. (2010). Synergistic antitumor effects of combined cathepsin B and cathepsin Z deficiencies on breast cancer progression and metastasis in mice. Proc. Nat. Acad. Sci. 107, 2497-2502.

Shaw, E. (1988). Peptidyl sulfonium salts. A new class of protease inhibitors. J. Biol. Chem. 263, 2768-2772.

Verdoes, M., Edgington, L.E., Scheeren, F.A., Leyva, M., Blum, G., Weiskopf, K., Bachmann, M.H., Ellman, J.A., and Bogyo, M. (2012). A Nonpeptidic Cathepsin S Activity-Based Probe for Noninvasive Optical Imaging of Tumor-Associated Macrophages. Chem. Biol. 19, 619-628.

Verdoes, M., Oresic Bender, K., Segal, E., van der Linden, W.A., Syed, S., Withana, N.P., Sanman, L.E., and Bogyo, M. (2013). Improved Quenched Fluorescent Probe for Imaging of Cysteine Cathepsin Activity. J. Am. Chem. Soc. 135, 14726-14730.

Wendt, W., Zhu, X.-R, L&uuml;bbert, H., and Sticliel, C.C. (2007). Differential expression of cathepsin X in aging and pathological central nervous system of mice. Exp. Neurol. 204, 525-540.

Further embodiments of the invention relate to:

1. A compound of formula I

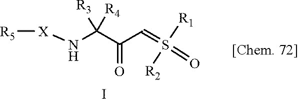

or a salt thereof, wherein
R₁ is selected from the group consisting of alkyl, (C₁-C₈) hydroxyalkyl, (C₁-C₈) haloalkyl, (C₃-C₈) cycloalkyl, (C₂-C₈) alkenyl and (C₂-C₈) alkynyl;
R₂ is selected from the group consisting of (C₁-C₈) alkyl, (C₁-C₈) hydroxyalkyl, (C₁-C₈) haloalkyl, (C₃-C₈) cycloalkyl, (C₂-C₈) alkenyl and (C₂-C₈) alkynyl;
R₃ is the sidechain of an alpha amino acid;
R₄ is selected from the group consisting of hydrogen and (C₁-C₄) alkyl;
R₅ is selected from the group consisting of a detectable element, an amine protecting group, (C₁-C₈) alkyl, (C₁-C₈) hydroxyalkyl, (C₁-C₈) haloalkyl, (C₃-C₈) cycloalkyl, (C₂-Cs) alkenyl,
(C₂-C₈) alkynyl, (C₁-C₈) alkylcarbonyl, (C₁-C₈) hydroxyalkylcarbonyl, (C₁-C₈) haloalkylcarbonyl, (C₃-C₈) cycloalkylcarbonyl, (C₁-C₈) alkyloxycarbonyl, benzyloxycarbonyl, and hydrogen;
X is
   (i) a bond; or
   (ii) a biradical moiety of formula II or III which is connected to the R₅ substituent via the amino group

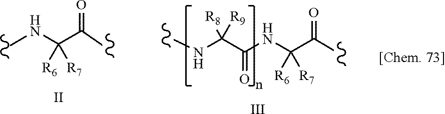

wherein
R₆ is the sidechain of an alpha amino acid;
R₇ is selected from the group consisting of hydrogen and (C₁-C₄) alkyl;
R₈ is the sidechain of an alpha amino acid;
R₉ is selected from the group consisting of hydrogen and (C₁-C₄) alkyl; and n is 1, 2, 3, or 4.

2. The compound of item 1, wherein R₃ is the sidechain of a natural alpha amino acid, or a structural isomer, homologue and/or structural analogue of said sidechain,
wherein said sidechain or structural isomer, homologue and/or structural analogue thereof is optionally substituted by an amine protecting group or a detectable element and optionally further substituted by one or more, same or different substituents R₃ₓ, wherein
R₃ₓ is selected from the group consisting of hydroxy, halogen, (C₁-C₄) alkyl, (C₁-C₄) hydroxyalkyl, (C₁-C₄) haloalkyl, (C₁-C₄) alkoxy, and (C₁-C₄) haloalkoxy.

3. The compound of item 1, wherein R₃ is the sidechain of a proteinogenic alpha amino acid, or a structural isomer, homologue and/or structural analogue of said sidechain,
wherein said sidechain or structural isomer, homologue and/or structural analogue thereof is optionally substituted by an amine protecting group or a detectable element and optionally further substituted by one or more, same or different substituents R₃ₓ, wherein
R₃ₓ is selected from the group consisting of hydroxy, halogen, (C₁-C₄) alkyl, (C₁-C₄) hydroxyalkyl, (C₁-C₄) haloalkyl, (C₁-C₄) alkoxy, and (C₁-C₄) haloalkoxy.

4. The compound of item 1, wherein R₃ is the sidechain of a proteinogenic alpha amino acid except lysine, or a structural isomer or homologue of said sidechain,
wherein said sidechain or structural isomer or homologue thereof is optionally substituted by an amine protecting group or a detectable element and optionally further substituted by one or more, same or different substituents R₃ₓ, wherein
R₃ₓ is selected from the group consisting of hydroxy, halogen, (C₁-C₄) alkyl, (C₁-C₄) hydroxyalkyl, (C₁-C₄) haloalkyl, (C₁-C₄) alkoxy, and (C₁-C₄) haloalkoxy.

5. The compound of item 1, wherein R₃ is the sidechain of an alpha amino acid selected from the group consisting of glycine, alanine, alpha-aminobutyric acid, valine, norvaline, leucine, isoleucine, norleucine, homonorleucine, methionine, ethionine, phenylalanine, tyrosine, levodopa, tryptophan, cysteine, homocysteine, selenocysteine, selenohomocysteine, selenomethionine, selenoethionine, lysine, histidine, arginine, ornithine, aspartic acid, glutamic acid, serine, homoserine, O-methyl-homoserine, O-ethyl-homoserine, threonine, asparagine, and glutamine, or a structural isomer, homologue and/or structural analogue of said sidechain,
wherein said sidechain or structural isomer, homologue and/or structural analogue thereof is optionally substituted by an amine protecting group or a detectable element and optionally further substituted by one or more, same or different substituents R₃ₓ, wherein
R₃ₓ is selected from the group consisting of hydroxy, halogen, (C₁-C₄) alkyl, (C₁-C₄) hydroxyalkyl, (C₁-C₄) haloalkyl, (C₁-C₄) alkoxy, and (C₁-C₄) haloalkoxy.

6. The compound of item 1, wherein R₃ is the sidechain of an alpha amino acid selected from the group consisting of glycine, alanine, alpha-aminobutyric acid, valine, norvaline, leucine, isoleucine, norleucine, homonorleucine, methionine, ethionine, phenylalanine, tyrosine, levodopa, tryptophan, cysteine, homocysteine, selenocysteine, selenohomocysteine, selenomethionine, selenoethionine, histidine, arginine, omithine, aspartic acid, glutamic acid, serine, homoserine, O-methyl-homoserine, O-ethyl-homoserine, threonine, asparagine, and glutamine, or a structural isomer or homologue of said sidechain,
wherein said sidechain or structural isomer or homologue thereof is optionally substituted by an amine protecting group or a detectable element and optionally further substituted by one or more, same or different substituents R₃ₓ, wherein
R₃ₓ is selected from the group consisting of hydroxy, halogen, (C₁-C₄) alkyl, (C₁-C₄) hydroxyalkyl, (C₁-C₄) haloalkyl, (C₁-C₄) alkoxy, and (C₁-C₄) haloalkoxy.

7. The compound of item 6, wherein the alpha amino acid is selected from the group consisting of glycine, alanine, alpha-aminobutyric acid, valine, norvaline, leucine, isoleucine, norleucine, homonorleucine, methionine, ethionine, phenylalanine, tyrosine, levodopa, tryptophan, cysteine, homocysteine, selenocysteine, selenohomocysteine, selenomethionine, and selenoethionine.

8. The compound of item 6, wherein the alpha amino acid is selected from the group consisting of alanine, alpha-aminobutyric acid, valine, norvaline, leucine, isoleucine, norleucine, homonorleucine, phenylalanine, and tryptophan.

9. The compound of item 6, wherein the alpha amino acid is selected from the group consisting of valine, norvaline, leucine, isoleucine, norleucine, and homonorleucine.

10. The compound of item 6, wherein the alpha amino acid is selected from the group consisting of valine, leucine, isoleucine, and norleucine.

11. The compound of item 6, wherein the alpha amino acid is leticine or norleucine.

12. The compound of item 6, wherein the alpha amino acid is norleucine.

13. The compound of item 1, wherein R₃ is selected from the group consisting of hydrogen, (C₁-C₈) alkyl, (C₁-C₈) hydroxyalkyl, (C₁-C₈) haloalkyl, (C₃-C₈) cycloalkyl, (C₂-C₈) alkenyl, (C₂-C₈) alkynyl, (C₆-C₁₀) arylmethyl, (C₃-C₉) heteroarylmethyl, and -CH₂CH₂CH₂CH₂N(R₃ₐ)(R_{3b}); wherein
R₃ₐ is selected from the group consisting of a detectable element, an amine protecting group, hydrogen, and (C₁-C₈) alkyl; and
R_{3b} is selected from the group consisting of hydrogen and (C₁-C₄) alkyl.

14. The compound of item 13, wherein R₃ is selected from the group consisting of (C₁-C₈) alkyl, (C₂-C₈) alkenyl, (C₂-C₈) alkynyl, (C₆-C₁₀) arylmethyl, (C₃-C₉) heteroarylmethyl, and -CH₂CH₂CH₂CH₂N(R₃ₐ)(R_{3b}).

15. The compound of item 13, wherein R₃ is selected from the group consisting of (Ci-Cs) alkyl, (C₆-C₁₀) arylmethyl, (C₃-C₉) heteroarylmethyl, and -CH₂CH₂CH₂CH₂N(R₃ₐ)(R_{3b}).

16. The compound of item 13, wherein R₃ is selected from the group consisting of (C₁-C₈) alkyl, benzyl, (lH-indol-3-yl) methyl, and -CH₂CH₂CH₂CH₂N(R₃ₐ)(R_{3b}).

17. The compound of item 13, wherein R₃ is selected from the group consisting of (C₁-C₆) alkyl, benzyl, (1H-indol-3-yl) methyl, and -CH₂CH₂CH₂CH₂N(R₃ₐ)(R_{3b}).

18. The compound of item 13, wherein R₃ is selected from the group consisting of ethyl, n-propyl, iso-propyl, n-butyl, sec-butyl, iso-butyl, n-pentyl, benzyl, (lH-indol-3-yl) methyl, and -CH₂CH₂CH₂CH₂N(R₃ₐ)(R_{3b}).

19. The compound of any one of items 13 to 18, wherein if X is a bond, R₃ₐ is not an amine protecting group, hydrogen, or (C₁-C₈) alkyl.

20. The compound of any one of items 13 to 18, wherein R₃ₐ is a detectable element.

21. The compound of any one of items 13 to 18, wherein if X is a bond, R₃ is not -CH₂CH₂CH₂CH₂N(R₃ₐ)(R_{3b}).

22. The compound of item 1, wherein R₃ is selected from the group consisting of hydrogen, (C₁-C₈) alkyl, (C₁-C₈) hydroxyalkyl, (C₁-C₈) haloalkyl, (C₃-C₈) cycloalkyl, (C₂-C₈) alkenyl, (C₂-C₈) alkynyl, (C₆-C₁₀) arylmethyl, and (C₃-C₉) heteroarylmethyl.

23. The compound of item 22, wherein R₃ is selected from the group consisting of (C₁-C₈) alkyl, (C₂-C₈) alkenyl, (C₂-C₈) alkynyl, (C₆-C₁₀) arylmethyl, and (C₃-C₉) heteroarylmethyl.

24. The compound of item 22, wherein R₃ is selected from the group consisting of (C₁-C₈) alkyl, (C₆-C₁₀) arylmethyl, and (C₃-C₉) heteroarylmethyl.

25. The compound of item 22, wherein R₃ is selected from the group consisting of (C₁-C₈) alkyl, benzyl, and (1H-indol-3-yl) methyl.

26. The compound of item 22, wherein R₃ is selected from the group consisting of (C₁-C₆) alkyl, benzyl, and (1H-indol-3-yl) methyl.

27. The compound of item 22, wherein R₃ is selected from the group consisting of ethyl, n-propyl, iso-propyl, n-butyl, sec-butyl, iso-butyl, n-pentyl, benzyl, and (1H-indol-3-yl) methyl.

28. The compound of item 22, wherein R₃ is selected from the group consisting of n-butyl, sec-butyl, and iso-butyl.

29. The compound of item 22, wherein R₃ is n-butyl.

30. The compound of item 1, wherein R₃ is -CH₂CH₂CH₂CH₂N(R₃ₐ)(R_{3b}); wherein
R₃ₐ is selected from the group consisting of a detectable element, an amine protecting group, hydrogen, and (C₁-C₈) alkyl; and
R_{3b} is selected from the group consisting of hydrogen and (C₁-C₄) alkyl.

31. The compound of item 30, wherein if X is a bond, R₃ₐ is not an amine protecting group, hydrogen, or (C₁-C₈) alkyl.

32. The compound of any one of items 13 to 18 and 30, wherein R₃ₐ is hydrogen.

33. The compound of any one of items 13 to 18 and 30, wherein R₃ₐ is an amine protecting group.

34. The compound of any one of items 13 to 18 and 30, wherein R₃ₐ is (C₁-C₈) alkyl.

35. The compound of any one of items 13 to 20 and 30 to 34, wherein R_{3b} is hydrogen.

36. The compound of any one of items 1 to 35, wherein R₁ is (C₁-C₈) alkyl.

37. The compound of any one of items 1 to 36, wherein R₁ is methyl.

38. The compound of any one of items 1 to 37, wherein R₂ is (C₁-C₈) alkyl.

39. The compound of any one of items 1 to 38, wherein R₂ is methyl.

40. The compound of any one of items 1 to 39, wherein R₄ is hydrogen.

41. The compound of any one of items 1 to 40, wherein R₅ is selected from the group consisting of a detectable element, an amine protecting group, and hydrogen.

42. The compound of any one of items 1 to 41, wherein R₆ is the sidechain of a natural alpha amino acid, or a structural isomer, homologue and/or structural analogue of said sidechain,
wherein said sidechain or structural isomer, homologue and/or structural analogue thereof is optionally substituted by an amine protecting group or a detectable element and optionally further substituted by one or more, same or different substituents R₆ₓ, wherein
R₆ₓ is selected from the group consisting of hydroxy, halogen, (C₁-C₄) alkyl, (C₁-C₄) hydroxyalkyl, (C₁-C₄) haloalkyl, (C₁-C₄) alkoxy, and (C₁-C₄) haloalkoxy.

43. The compound of any one of items 1 to 41, wherein R₆ is the sidechain of a proteinogenic alpha amino acid, or a structural isomer, homologue and/or structural analogue of said sidechain,
wherein said sidechain or structural isomer, homologue and/or structural analogue thereof is optionally substituted by an amine protecting group or a detectable element and optionally further substituted by one or more, same or different substituents R₆ₓ, wherein
R₆ₓ is selected from the group consisting of hydroxy, halogen, (C₁-C₄) alkyl, (C₁-C₄) hydroxyalkyl, (C₁-C₄) haloalkyl, (C₁-C₄) alkoxy, and (C₁-C₄) haloalkoxy.

44. The compound of any one of items 1 to 41, wherein R₆ is the sidechain of an alpha amino acid selected from the group consisting of glycine, alanine, alpha-aminobutyric acid, valine, norvaline, leucine, isoleucine, norleucine, homonorleucine, methionine, ethionine, phenylalanine, tyrosine, levodopa, tryptophan, cysteine, homocysteine, selenocysteine, selenohomocysteine, selenomethionine, selenoethionine, lysine, histidine, arginine, omithine, aspartic acid, glutamic acid, serine, homoserine, O-methyl-homoserine, O-ethyl-homoserine, threonine, asparagine, and glutamine, or a structural isomer, homologue and/or structural analogue of said sidechain,
wherein said sidechain or structural isomer, homologue and/or structural analogue thereof is optionally substituted by an amine protecting group or a detectable element and optionally further substituted by one or more, same or different substituents R₆ₓ wherein
R₆ₓ is selected from the group consisting of hydroxy, halogen, (C₁-C₄) alkyl, (C₁-C₄) hydroxyalkyl, (C₁-C₄) haloalkyl, (C₁-C₄) alkoxy, and (C₁-C₄) haloalkoxy.

45. The compound of item 44, wherein the alpha amino acid is phenylalanine.

46. The compound of any one of items 1 to 41, wherein R₆ is the sidechain of phenylalanine, or a structural isomer, homologue and/or structural analogue of said sidechain.

47. The compound of any one of items 1 to 41, wherein R₆ is the sidechain of phenylalanine.

48. The compound of any one of items 1 to 47, wherein R₇ is hydrogen.

49. The compound of any one of items 1 to 48, wherein R₈ is the sidechain of a natural alpha amino acid, or a structural isomer, homologue and/or structural analogue of said sidechain,
wherein said sidechain or structural isomer, homologue and/or structural analogue thereof is optionally substituted by an amine protecting group or a detectable element and optionally further substituted by one or more, same or different substituents R₈ₓ, wherein
R₈ₓ is selected from the group consisting of hydroxy, halogen, (C₁-C₄) alkyl, (C₁-C₄) hydroxyalkyl, (C₁-C₄) haloalkyl, (C₁-C₄) alkoxy, and (C₁-C₄) haloalkoxy.

50. The compound of any one of items 1 to 48, wherein R₈ is the sidechain of a proteinogenic alpha amino acid, or a structural isomer, homologue and/or structural analogue of said sidechain,
wherein said sidechain or structural isomer, homologue and/or structural analogue thereof is optionally substituted by an amine protecting group or a detectable element and optionally further substituted by one or more, same or different substituents R₈ₓ, wherein
R₈ₓ is selected from the group consisting of hydroxy, halogen, (C₁-C₄) alkyl, (C₁-C₄) hydroxyalkyl, (C₁-C₄) haloalkyl, (C₁-C₄) alkoxy, and (C₁-C₄) haloalkoxy.

51. The compound of any one of items 1 to 48, wherein R₈ is the sidechain of an alpha amino acid selected from the group consisting of glycine, alanine, alpha-aminobutyric acid, valine, norvaline, leucine, isoleucine, norleucine, homonorleucine, methionine, ethionine, phenylalanine, tyrosine, levodopa, tryptophan, cysteine, homocysteine, selenocysteine, selenohomocysteine, selenomethionine, selenoethionine, lysine, histidine, arginine, ornithine, aspartic acid, glutamic acid, serine, homoserine, O-methyl-homoserine, O-ethyl-homoserine, threonine, asparagine, and glutamine, or a structural isomer, homologue and/or structural analogue of said sidechain, wherein said sidechain or structural isomer, homologue and/or structural analogue thereof is optionally substituted by an amine protecting group or a detectable element and optionally further substituted by one or more, same or different substituents R₈ₓ, wherein R₈ₓ is selected from the group consisting of hydroxy, halogen, (C₁-C₄) alkyl, (C₁-C₄) hydroxyalkyl, (C₁-C₄) haloalkyl, (C₁-C₄) alkoxy, and (C_{J}-C₄) haloalkoxy.

52. The compound of any one of items 1 to 51, wherein R₉ is hydrogen.

53. The compound of any one of items 1 to 52 wherein the amine protecting group is selected from the group consisting of benzyloxycarbonyl (Cbz), 9-fluorenylmethyloxycarbonyl (Fmoc), tert-butyloxycarbonyl (Boc), allyloxycarbonyl (Alloc), p-toluenesulfonyl (Tos), 2,2,5_{.}7,8-pentanietliylchronian-6-sulfonyt (Pmc), 2,2,4,6,7-pentamethyl-2,3-dihydrobenzofuran-5-su]fonyl (Pbf), mesityl-2-sulfonyl (Mts), 4-methox_{N,}-2,3,6-trimethylphenylsulfonyl (Mtr), acetamido, and phthalimido.

54. The compound of any one of items 1 to 53 wherein the amine protecting group is benzyloxycarbonyl.

55. The compound of any one of items 1 to 54 having the formula IA:

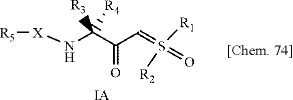

56. The compound of any one of items 1 to 55, wherein the biradical moiety has the formula IIA or IIIA

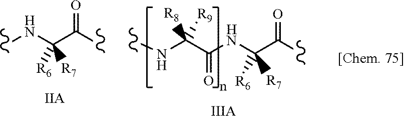

57. The compound of any one of items 1 to 55, wherein X is a bond.

58. The compound of any one of items 1 to 55, wherein X is a biradical moiety of formula II.

59. The compound of any one of items 1 to 55, wherein X is a biradical moiety of formula III.

60. The compound of item 56, wherein X is a biradical moiety of formula IIA.

61. The compound of item 56, wherein X is a biradical moiety of formula IIIA.

62. The compound of any one of items 1 to 61, wherein n is 1.

63. The compound of any one of items 1 to 62, wherein the detectable element is selected from the group consisting of a fluorescent label, a biotin label, a radiolabel, a chelator, and a bioorthogonal ligation handle.

64. The compound of any one of items 1 to 63, wherein the detectable element is a fluorescent label.

65. The compound of item 64, wherein the fluorescent label is selected from the group consisting of a fluorescein, an Oregon green, a bora-diaza-indecene dye, a rhodamine dye, a benzopyrillium dye, a coumarin dye, a cyanine label or a benzoindole label.

66. The compound of item 64, wherein the fluorescent label is a cyanine label.

67. The compound of item 64, wherein the fluorescent label is a cyanine label having a formula selected from the following group of formulas:

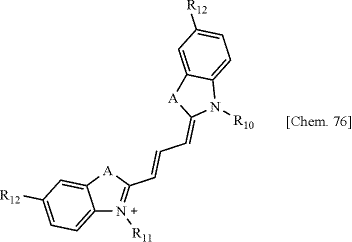

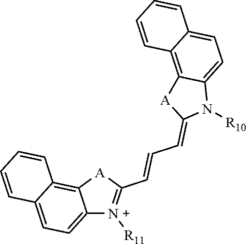

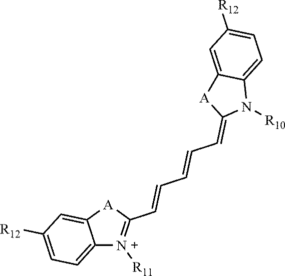

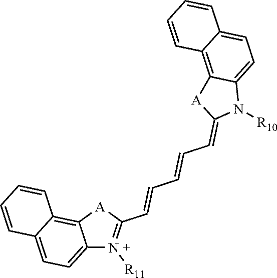

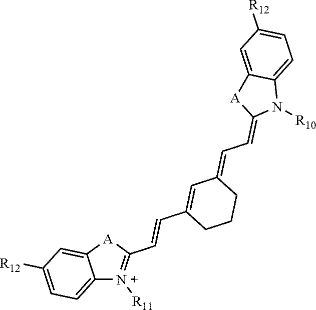

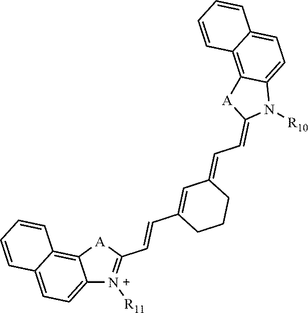

wherein in each of the above formulas,
A is selected from the group consisting of CHz, C(CH₃)₂, C(C₂H₅)₂, NH, N(CH₃), N(C₂H₅), O, S, and Se;
Rᵢₒ is selected from the group consisting of $-(CH₂)ₚ-C(=O)-& and $-(CH₂)_{q}-C(=0)-NH-[CH2CH₂O]r-CH₂CH₂-C(=O)-_{&};
wherein
p is 2, 3, 4, 5, 6, 7, or 8;
q is 2, 3, 4, 5, 6, 7, or 8;
r is 2, 3, 4, 5, 6, 7, or 8;
$ represents the point of connection to the nitrogen atom of the cyanine moiety;
and & represents the point of connection to the remainder of the molecule;
R₁₁ is selected from the group consisting of (Ci-C₈)alkyl, and (C_{f,}-Cio)aryl; and
R₁₂ is H or a sulfo group.

68. The compound of item 67, wherein
A is selected from the group consisting of CH2, C(CH₃)₂, and C(C₂Hs)2;
Rᵢₒ is selected from the group consisting ₒf $-(CH₂)ₚ-C(=O)-& and $-(CH₂)_{q}-C(=O)-NH-[CH₂CH₂O]t-CH₂CH₂-C(=O)-&;
wherein
p is 2, 3, 4, 5, or 6;
q is 2, 3, 4, 5, or 6;
r is 2, 3, 4, 5, or 6;
$ represents the point of connection to the nitrogen atom of the cyanine moiety;
and & represents the point of connection to the remainder of the molecule;
Ri ₁ is (C₁-C₈)alkyl; and
R₁₂ is H or a sulfo group.

69. The compound of item 67, wherein
A is C(CH₃)₂ or C(C₂H₅)₂;
R₁₀ is selected from the group consisting of $-(CH₂)ₚ-C(-0)-& and $-(CH₂)_{q}-C(=O)-NH-[CH₂CH₂0 ]ᵣ-CH₂CH₂-C(’ooO )-&;
wherein
p is 2, 3, 4, 5, or 6;
q is 2, 3, 4, 5, or 6;
r is 2, 3, 4, 5, or 6;
$ represents the point of connection to the nitrogen atom of the cyanine moiety;
and & represents the point of connection to the remainder of the molecule;
R₁₁ is methyl, ethyl or propyl; and
R₁₂ is H or a sulfo group.

70. The compound of item 67, wherein
A is C(CH₃)₂;
R₁₀ is selected from the group consisting of $-(CH₂)ₚ-C(=O)-& and $-(CH₂)_{q}-C(=0)-NH-[CH2CH₂O]r-CH₂CH₂-C(=O)-&;
wherein
p is 4, 5, or 6;
q is 4, 5, or 6;
r is 3, 4, 5, or 6;
$ represents the point of connection to the nitrogen atom of the cyanine moiety;
and & represents the point of connection to the remainder of the molecule;
R₁₁ is methyl or ethyl; and
R₁₂ is H or a sulfo group.

71. The compound of item 67, wherein
A is C(CH₃)₂;
R.ₒ is $-(CH₂)ₚ-C(:=O)-&; wherein
p is 4, 5, or 6; and
$ represents the point of connection to the nitrogen atom of the cyanine moiety;
and & represents the point of connection to the remainder of the molecule;
R₁₁ is methyl or ethyl; and
R₁₂ is a sulfo group.

72. The compound of item 67, wherein
A is C(CH₃)₂;
R₁₀ is $-(CH₂)_{q}-C(=O)-NH-[CH₂CH₂O]r-CH₂CH₂-C(=O)-&;
wherein
q is 4, 5, or 6;
r is 3, 4, 5, or 6;
$ represents the point of connection to the nitrogen atom of the cyanine moiety;
and & represents the point of connection to the remainder of the molecule;
R₁₁ is methyl or ethyl; and
R₁₂ is H.

73. The compound of any one of items 67 to 72, wherein p is 5, q is 5 and r is 4.

74. The compound of item 64, wherein the fluorescent label is a cyanine label having a formula selected from the following group of formulas:

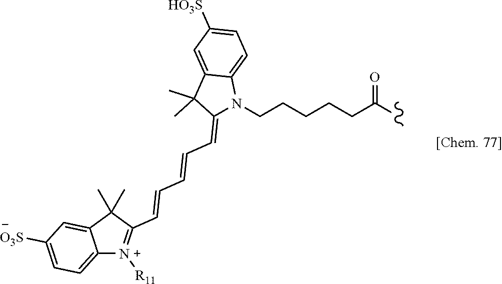

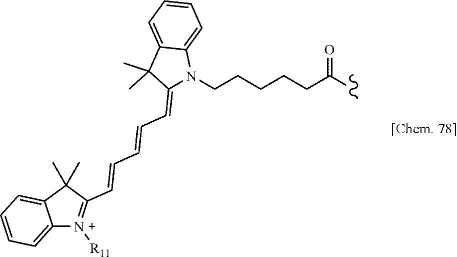

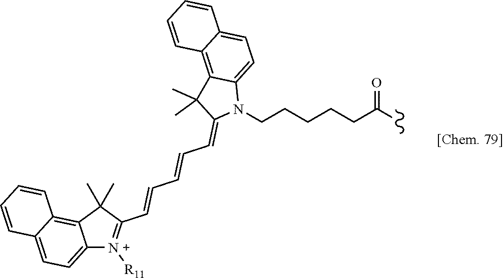

wherein in each of the above formulas,
the curled line represents the point of connection to the remainder of the molecule; and R
ll is selected from the group consisting of (C₁-C₈)alkyl, and (C₆-C₁₀)aryl.

75. The compound of item 74, wherein R₁₁ is (C₁-C₈)alkyl.

76. The compound of item 74, wherein R₁₁ is methyl or ethyl.

77. The compound of item 64, wherein the fluorescent label is a cyanine label having the formula

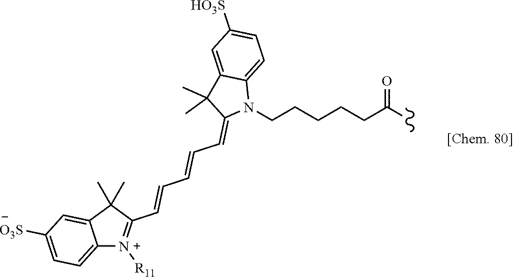

wherein the curled line represents the point of connection to the remainder of the molecule; and R₁₁ is methyl or ethyl.

78. The compound of any one of items 1 to 77, wherein the compound comprises at least one detectable element.

79. The compound of any one of items 1 to 77, wherein the compound comprises one, two or three detectable elements.

80. The compound of any one of items 1 to 77, wherein the compound comprises one detectable element.

81. The compound of any one of items 1 to 80, wherein R₅ is a detectable element.

82. The compound of any one of items 1 to 81, wherein R₃ bears a detectable element.

83. The compound of any one of items 1 to 82, wherein R₃ is the sidechain of lysine, or a structural isomer, homologue and/or structural analogue of said sidechain, wherein said sidechain or structural isomer, homologue and/or structural analogue thereof is substituted by a detectable element and optionally further substituted by one or more, same or different substituents R₃ₓ, wherein R₃ₓ is selected from the group consisting of hydroxy, halogen, (C₁-C₄) alkyl, (C₁-C₄) hydroxyalkyl, (C₁-C₄) haloalkyl, (C₁-C₄) alkoxy, and (C₁-C₄) haloalkoxy.

84. The compound of any one of items 1 to 82,
wherein R₃ is -CH₂CH₂CH₂CH₂N(R₃ₐ)(R_{3b}); wherein
R;ₐ is a detectable element; and
R_{3b} is selected from the group consisting of hydrogen and (C₁-C₄) alkyl.

85. The compound of any one of items 82 to 84, wherein R₅ is selected from the group consisting of an amine protecting group, (C₁-C₈) alkyl, (C₁-C₈) hydroxyalkyl, (C₁-C₈) haloalkyl, (C₃-C₈) cycloalkyl, (C₂-C₈) alkenyl, (C₂-C₈) alkynyl, (C₁-C₈) alkylcarbonyl, (C₁-C₈) hydroxyalkylcarbonyl, (C₁-C₈) haloalkylcarbonyl, (C₃-Cs) cycloalkylcarbonyl, (C₁-C₈) alkyloxycarbonyl, benzyloxycarbonyl, and hydrogen.

86. The compound of any one of items 82 to 84, wherein R₅ is an amine protecting group.

87. The compound of any one of items 1 to 86, wherein X is a biradical moiety of formula II or IIA, and R₆ bears a detectable element.

88. The compound of any one of items 1 to 87, wherein X is a biradical moiety of formula II or IIA, and R₆ is the sidechain of lysine, or a structural isomer, homologue and/or structural analogue of said sidechain,
wherein said sidechain or structural isomer, homologue and/or structural analogue thereof is substituted by a detectable element and optionally further substituted by one or more, same or different substituents R₆ₓ, wherein R₆ₓ is selected from the group consisting of hydroxy, halogen, (C₁-C₄) alkyl, (C₁-C₄) hydroxyalkyl, (C₁-C₄) haloalkyl, (C₁-C₄) alkoxy, and (C₁-C₄) haloalkoxy.

89. The compound of any one of items 1 to 86, wherein X is a biradical moiety of formula III or IIIA, and R₆ bears a detectable element.

90. The compound of any one of items 1 to 86 and 89, wherein X is a biradical moiety of formula III or IIIA, and R₆ is the sidechain of lysine, or a structural isomer, homologue and/or structural analogue of said sidechain, wherein said sidechain or structural isomer, homologue and/or structural analogue thereof is substituted by a detectable element and optionally further substituted by one or more, same or different substituents R₆ₓ, wherein R₆ₓ is selected from the group consisting of hydroxy, halogen, (C₁-C₄) alkyl, (C₁-C₄) hydroxyalkyl, (C₁-C₄) haloalkyl, (C₁-C₄) alkoxy, and (C₁-C₄) haloalkoxy.

91. The compound of any one of items 1 to 86 and 89 to 90, wherein X is a biradical moiety of formula III or IIIA, and R₈ bears a detectable element.

92. The compound of any one of items 1 to 86 and 89 to 91, wherein X is a biradical moiety of formula III or IIIA, and R₈ is the sidechain of lysine, or a structural isomer, homologue and/or structural analogue of said sidechain, wherein said sidechain or structural isomer, homologue and/or structural analogue thereof is substituted by a detectable element and optionally further substituted by one or more, same or different substituents R₈ₓ, wherein R₈ₓ is selected from the group consisting of hydroxy, halogen, (C₁-C₄) alkyl, (C₁-C₄) hydroxyalkyl, (C₁-C₄) haloalkyl, (C₁-C₄) alkoxy, and (C₁-C₄) haloalkoxy.

93. A compound selected from the group of formulas consisting of:

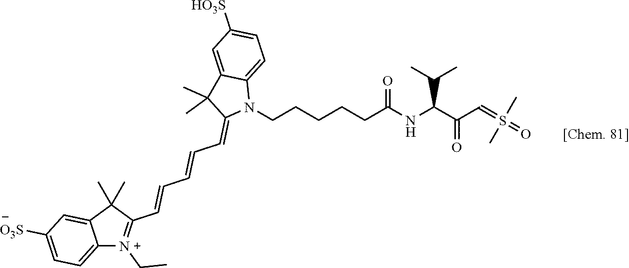

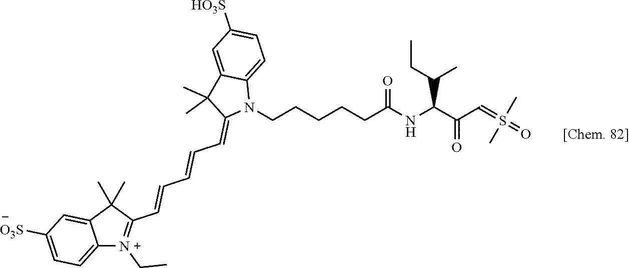

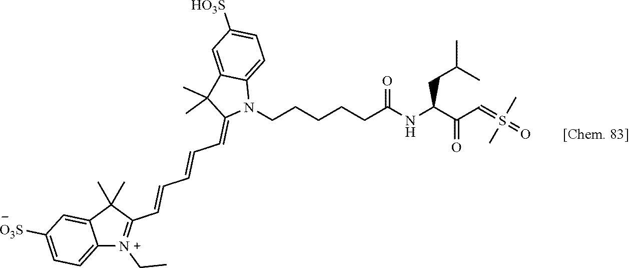

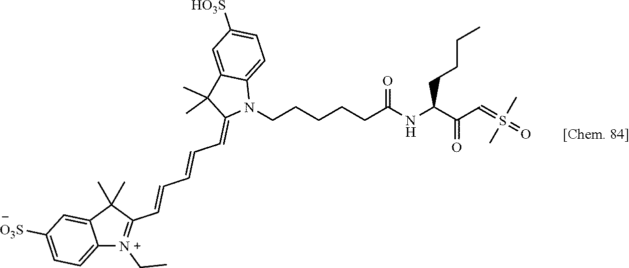

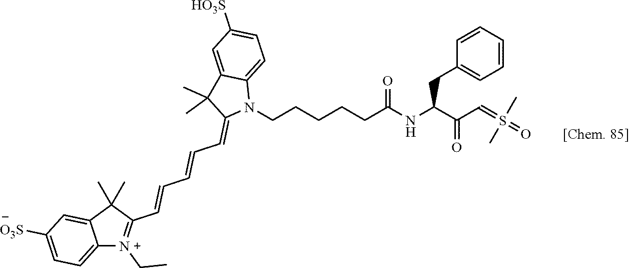

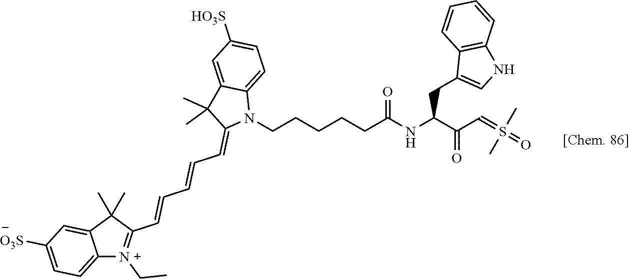

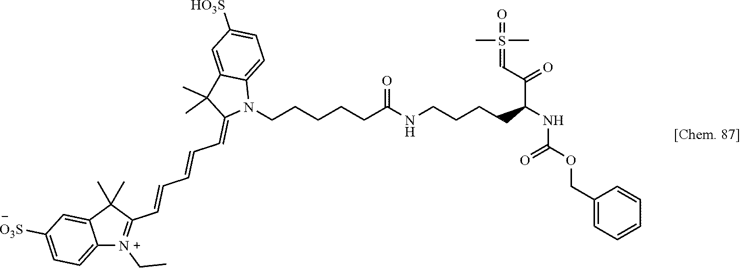

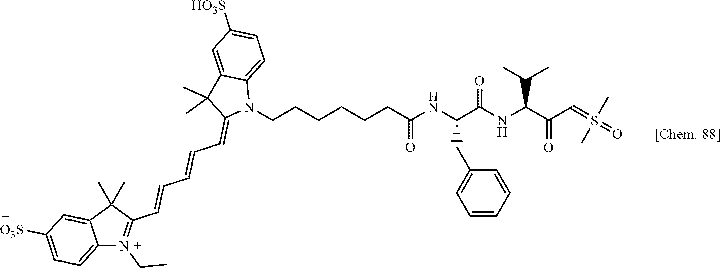

or a salt thereof.

94. A compound of formula:

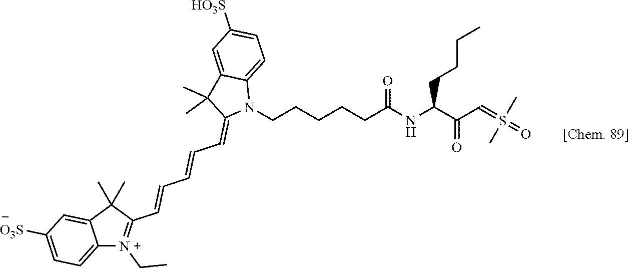

or a salt thereof.

95. A composition comprising a compound of any one of items 1 to 94 or a salt thereof, and an excipient.

96. The composition of item 95, wherein the composition comprises a compound of any one of items 78 to 94 or a salt thereof, and an excipient.

97. A method of detecting cysteine protease activity comprising
(1) contacting the cysteine protease with an activity-based probe compound comprising a sulfoxonium ylide moiety as warhead, and
(2) subsequently analyzing the cysteine protease comprising measuring a detectable signal.

98. The method of item 97, wherein the method is an in vitro method.

99. An in vitro method of detecting cysteine protease activity comprising
(1) contacting a biological sample with an activity-based probe compound comprising a sulfoxonium ylide moiety as warhead, and
(2) subsequently analyzing the biological sample comprising measuring a detectable signal.

100. A method of detecting cysteine protease activity in a biological sample obtained from a subject comprising
(1) contacting the biological sample in vitro with an activity-based probe compound comprising a sulfoxonium ylide moiety as warhead, and
(2) subsequently analyzing the biological sample comprising measuring a detectable signal.

101. The method of any one of items 97 to 100, wherein the sulfoxonium ylide moiety has the formula (IV)

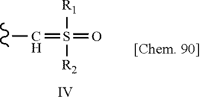

wherein
R₁ is selected from the group consisting of (C₁-C₈) alkyl, (C₁-C₈) hydroxyalkyl, (C₁-C₈) haloalkyl, (C₃-C₈) cycloalkyl, (C₂-C₈) alkenyl and (C₂-C₈) alkynyl; and
R₂ is selected from the group consisting of (C₁-C₈) alkyl, (C₁-C₈) hydroxyalkyl, (C₁-C₈) haloalkyl, (C₃-C₈) cycloalkyl, (C₂-C₈) alkenyl and (C₂-C₈) alkynyl.

102. The method of item 101, wherein R₁ is (C₁-C₈) alkyl, and R₂ is (C₁-C₈) alkyl.

103. The method of item 101, wherein R₁ is methyl and R₂ is methyl.

104. A method of detecting cysteine protease activity comprising
(1) contacting the cysteine protease with a compound of any one of items 78 to 94 or a salt thereof, or with a composition of item 96, and
(2) subsequently analyzing the cysteine protease comprising measuring a detectable signal.

105. The method of item 104, wherein the method is an in vitro method.

106. An in vitro method of detecting cysteine protease activity comprising
(1) contacting a biological sample with a compound of any one of items 78 to 94 or a salt thereof, or with a composition of item 96, and
(2) subsequently analyzing the biological sample comprising measuring a detectable signal.

107. A method of detecting cysteine protease activity in a biological sample obtained from a subject comprising
(1) contacting the biological sample in vitro with a compound of any one of items 78 to 94 or a salt thereof, or with a composition of item 96, and
(2) subsequently analyzing the biological sample comprising measuring a detectable signal.

108. The method of any one of items 97 to 107, wherein step (2) comprises performing at least one analytical method selected from the group consisting of gel electrophoresis and subsequent in-gel fluorescence, gel electrophoresis and subsequent radiography, gel electrophoresis and subsequent immunoblotting, fluorescent microscopy, flow cytometry, ex vivo optical imaging, radiography, affinity purification and subsequent mass spectrometry, and affinity purification and subsequent proteomics.

109. The method of any one of items 97 to 108, wherein the detectable signal is measured by fluorescence measurement.

110. The method of item 108 or 109, wherein the at least one analytical method is selected from the group consisting of gel electrophoresis and subsequent in-gel fluorescence, fluorescent microscopy, and flow cytometry.

111. The method of item 108 or 109, wherein the at least one analytical method is selected from gel electrophoresis and subsequent in-gel fluorescence, and fluorescent microscopy.

112. The method of any one of items 97 to 111, wherein said compound comprises a detectable element in the form of a fluorescent label.

113. The method of any one of items 109 to 111, wherein said compound comprises a detectable element in the form of a bioorthogonal ligation handle, and wherein step (2) comprises secondary labeling by click-chemistry to apply a fluorescent label prior to performing the at least one analytical method.

114. The method of any one of items 109 to 111, wherein said compound comprises a detectable element in the form of biotin, and wherein step (2) comprises secondary labeling with fluorescently tagged streptavidin or secondary labeling with a fluorescently tagged antibody specific for biotin, prior to performing the at least one analytical method.

115. The method of any one of items 108 to 114, wherein the gel electrophoresis is a one-dimensional or a two-dimensional gel electrophoresis.

116. The method of any one of items 108 to 115, wherein the gel electrophoresis is an SDS-PAGE.

117. The method of any one of items 99 to 103 and 106 to 116, wherein the biological sample is selected from the group consisting of cells, cell lysates, tissue samples, tissue lysates and bodily fluids.

118. The method of item 117, wherein the biological sample is a cell lysate or a tissue lysate.

119. The method of item 117, wherein the biological sample is a cleared cell lysate or a cleared tissue lysate.

120. The method of item 117, wherein the biological sample is live cells.

121. The method of item 120, wherein the live cells are lysed and cleared between step (1) and step (2).

122. The method of any one of items 99 to 103 and 106 to 121, wherein the biological sample is obtained from a human subject.

123. A method of detecting cysteine protease activity comprising
(1) administering to a subject an activity-based probe compound comprising a sulfoxonium ylide moiety as warhead,
(2) subsequently obtaining a biological sample from the subject; and
(3) subsequently analyzing the biological sample comprising measuring a detectable signal.

124. The method of item 123, wherein the activity-based probe compound is administered intravenously.

125. The method of item 123 or 124, wherein the sulfoxonium ylide moiety has the formula (IV)

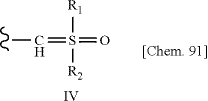

wherein
R₁ is selected from the group consisting of (C₁-C₈) alkyl, (C₁-C₈) hydroxyalkyl, (C₁-C₈) haloalkyl, (C₃-C₈) cycloalkyl, (C₂-C₈) alkenyl and (C₂-C₈) alkynyl; and
R₂ is selected from the group consisting of (C₁-C₈) alkyl, (C₁-C₈) hydroxyalkyl, (C₁-C₈) haloalkyl, (C₃-C₈) cycloalkyl, (C₁-C₈) alkenyl and (C₂-C₈) alkynyl.

126. The method of item 125, wherein R₁ is (C₁-C₈) alkyl, and R₂ is (C₁-C₈) alkyl.

127. The method of item 125, wherein R₁ is methyl and R₂ is methyl.

128. A method of detecting cysteine protease activity comprising
(1) administering to a subject a compound of any one of items 78 to 94 or a salt thereof, or a composition of item 96,
(2) subsequently obtaining a biological sample from the subject; and
(3) subsequently analyzing the biological sample comprising measuring a detectable signal.

129. The method of item 128, wherein the compound or salt thereof, or the composition, is administered intravenously.

130. The method of any one of items 123 to 129, wherein step (3) comprises performing at least one analytical method selected from the group consisting of gel electrophoresis and subsequent in-gel fluorescence, gel electrophoresis and subsequent radiography, gel electrophoresis and subsequent immunoblotting, fluorescent microscopy, flow cytometry, ex vivo optical imaging, radiography, affinity purification and subsequent mass spectrometry, and affinity purification and subsequent proteomics.

131. The method of any one of items 123 to 130, wherein the detectable signal is measured by fluorescence measurement.

132. The method of item 130 or 131, wherein the at least one analytical method is selected from the group consisting of gel electrophoresis and subsequent in-gel fluorescence, fluorescent microscopy, and flow cytometry.

133. The method of item 130 or 131, wherein the at least one analytical method is selected from gel electrophoresis and subsequent in-gel fluorescence, and fluorescent microscopy.

134. The method of any one of items 123 to 133, wherein said compound comprises a detectable element in the form of a fluorescent label.

135. The method of any one of items 131 to 133, wherein said compound comprises a detectable element in the form of a bioorthogonal ligation handle, and wherein step (3) comprises secondary labeling by click-chemistry to apply a fluorescent label prior to performing the at least one analytical method.

136. The method of any one of items 131 to 133, wherein said compound comprises a detectable element in the form of biotin, and wherein step (3) comprises secondary labeling with fluorescently tagged streptavidin or secondary labeling with a fluorescently tagged antibody specific for biotin, prior to performing the at least one analytical method.

137. The method of any one of items 130 to 136, wherein the gel electrophoresis is a one-dimensional or a two-dimensional gel electrophoresis.

138. The method of any one of items 130 to 137, wherein the gel electrophoresis is an SDS-PAGE.

139. The method of any one of items 123 to 138, wherein the biological sample is selected from the group consisting of cells, cell lysates, tissue samples, tissue lysates and bodily fluids.

140. The method of item 139, wherein the biological sample is a cell lysate or a tissue lysate.

141. The method of item 139, wherein the biological sample is a cleared cell lysate or a cleared tissue lysate.

142. The method of any one of items 123 to 141, wherein the subject is a human subject.

143. An in vivo method of detecting cysteine protease activity in a subject comprising
(1) administering to the subject an activity-based probe compound comprising a sulfoxonium ylide moiety as warhead, and
(2) subsequently examining the subject comprising measuring a detectable signal.

144. The method of item 143, wherein the activity-based probe compound is administered intravenously.

145. The method of item 143 or 144, wherein the sulfoxonium ylide moiety has the formula (IV)

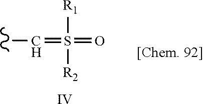

wherein
R₁ is selected from the group consisting of alkyl, (C₁-C₈) hydroxyalkyl, (C₁-C₈) haloalkyl, (C₃-C₈) cycloalkyl, (C₂-C₈) alkenyl and (C₂-C₈) alkynyl; and
R₂ is selected from the group consisting of (C₁-C₈) alkyl, (C₁-C₈) hydroxyalkyl, (C₁-C₈) haloalkyl, (C₃-C₈) cycloalkyl, (C₂-C₈) alkenyl and (C₂-C₈) alkynyl.

146. The method of item 145, wherein R₁ is (C₁-C₈) alkyl, and R₂ is (C₁-C₈) alkyl.

147. The method of item 145, wherein R₁ is methyl and R₂ is methyl.

148. An in vivo method of detecting cysteine protease activity in a subject comprising
(1) administering to the subject a compound of any one of items 78 to 94 or a salt thereof, or a composition of item 96, and
(2) subsequently examining the subject comprising measuring a detectable signal.

149. The method of item 148, wherein the compound or salt thereof, or the composition, is administered intravenously.

150. The method of any one of items 143 to 149, wherein the detectable signal is measured by in vivo optical imaging, radiography, or positron emission tomography.

151. The method of any one of items 143 to 150, wherein the subject is a human subject.

152. The method of any one of items 97 to 151, wherein the cysteine protease is a mammalian cysteine protease.

153. The method of any one of items 97 to 151, wherein the cysteine protease is a human cysteine protease.

154. The method of any one of items 97 to 151, wherein the cysteine protease is a cysteine cathepsin.

155. The method of any one of items 97 to 151, wherein the cysteine protease is a mammalian cysteine cathepsin.

156. The method of any one of items 97 to 151, wherein the cysteine protease is a human cysteine cathepsin.

157. The method of any one of items 97 to 151, wherein the cysteine protease is cathepsin X.

158. The method of any one of items 97 to 151, wherein the cysteine protease is mammalian cathepsin X.

159. The method of any one of items 97 to 151, wherein the cysteine protease is human cathepsin X.

160. The method of any one of items 97 to 159, wherein cathepsin X activity is detected and cathepsin B activity and/or cathepsin L activity are not detected.

161. The method of any one of items 97 to 159, wherein cathepsin X activity and cathepsin S activity are detected and cathepsin B activity and/or cathepsin L activity are not detected.

162. A method of inhibiting a cysteine protease comprising contacting the cysteine protease with a compound of any one of items 1 to 94 or a salt thereof, or with a composition of item 95.

163. The method of item 162, wherein the method is an in vitro method.

164. An in vitro method of inhibiting a cysteine protease comprising contacting a biological sample with a compound of any one of items 1 to 94 or a salt thereof, or with a composition of item 95.

165. A method of inhibiting a cysteine protease in a biological sample obtained from a subject comprising contacting the biological sample in vitro with a compound of any one of items 1 to 94 or a salt thereof, or with a composition of item 95.

166. The method of item 164 or 165, wherein the biological sample is selected from the group consisting of cells, cell lysates, tissue samples, tissue lysates and bodily fluids.

167. An in vivo method of inhibiting a cysteine protease in a subject comprising administering to the subject a compound of any one of items 1 to 94 or a salt thereof, or a composition of item 95.

168. The method of item 167, wherein the compound or salt thereof, or the composition, is administered intravenously.

169. The method of any one of items 162 to 168, wherein the cysteine protease is a mammalian cysteine protease.

170. The method of any one of items 162 to 168, wherein the cysteine protease is a human cysteine protease.

171. The method of any one of items 162 to 168, wherein the cysteine protease is a cysteine cathepsin.

172. The method of any one of items 162 to 168, wherein the cysteine protease is a mammalian cysteine cathepsin.

173. The method of any one of items 162 to 168, wherein the cysteine protease is a human cysteine cathepsin.

174. The method of any one of items 162 to 168, wherein the cysteine protease is cathepsin X.

175. The method of any one of items 162 to 168, wherein the cysteine protease is mammalian cathepsin X.

176. The method of any one of items 162 to 168, wherein the cysteine protease is human cathepsin X.

177. The method of any one of items 162 to 176, wherein cathepsin X is inhibited and cathepsin B and/or cathepsin L are not inhibited.

178. The method of any one of items 162 to 176, wherein cathepsin X and cathepsin S are inhibited and cathepsin B and/or cathepsin L are not inhibited.

179. A method of diagnosing a disease associated with a cysteine protease activity in a subject comprising
(1) contacting a biological sample obtained from the subject in vitro with a compound of any one of items 78 to 94 or a salt thereof, or with a composition of item 96, and
(2) subsequently analyzing the biological sample comprising measuring a detectable signal.

180. The method of item 179, wherein the method comprises detecting cysteine protease activity according to the method of any one of items 107 to 122 and 152 to 161.

181. A method of diagnosing a disease associated with a cysteine protease activity in a subject comprising
(1) administering to the subject a compound of any one of items 78 to 94 or a salt thereof, or a composition of item 96,
(2) subsequently obtaining a biological sample from the subject; and
(3) subsequently analyzing the biological sample comprising measuring a detectable signal.

182. The method of item 181, wherein the method comprises detecting cysteine protease activity according to the method of any one of items 128 to 142 and 152 to 161.

183. An in vivo method of diagnosing a disease associated with a cysteine protease activity in a subject comprising
(1) administering to the subject a compound of any one of items 78 to 94 or a salt thereof, or a composition of item 96, and
(2) subsequently examining the subject comprising measuring a detectable signal.

184. The method of item 183, wherein the method comprises detecting cysteine protease activity according to the method of any one of items 148 to 161.

185. A compound of any one of items 78 to 94 or a salt thereof, for use in a method of diagnosing a disease associated with a cysteine protease activity in a subject, wherein the method comprises
(1) administering to the subject a compound of any one of items 78 to 94 or a salt thereof,
(2) subsequently obtaining a biological sample from the subject; and
(3) subsequently analyzing the biological sample comprising measuring a detectable signal.

186. The compound for use of item 185, wherein the method comprises detecting cysteine protease activity according to the method of any one of items 128 to 142 and 152 to 161.

187. A composition of item 96 for use in a method of diagnosing a disease associated with a cysteine protease activity in a subject, wherein the method comprises
(1) administering to the subject a composition of item 96,
(2) subsequently obtaining a biological sample from the subject; and
(3) subsequently analyzing the biological sample comprising measuring a detectable signal.

188. The composition for use of item 187, wherein the method comprises detecting cysteine protease activity according to the method of any one of items 128 to 142 and 152 to 161.

189. A compound of any one of items 78 to 94 or a salt thereof, for use in an in vivo method of diagnosing a disease associated with a cysteine protease activity in a subject, wherein the method comprises
(1) administering to the subject a compound of any one of items 78 to 94 or a salt thereof, and
(2) subsequently examining the subject comprising measuring a detectable signal.

190. The compound for use of item 189, wherein the method comprises detecting cysteine protease activity according to the method of any one of items 148 to 161.

191. A composition of item 96 for use in an in vivo method of diagnosing a disease associated with a cysteine protease activity in a subject wherein the method comprises
(1) administering to the subject a composition of item 96, and
(2) subsequently examining the subject comprising measuring a detectable signal.

192. The composition for use of item 191, wherein the method comprises detecting cysteine protease activity according to the method of any one of items 148 to 161.

193. A method of treating a disease associated with a cysteine protease activity comprising administering to a patient in need thereof a therapeutically effective amount of a compound of any one of items 1 to 94 or a salt thereof, or a therapeutically effective amount of a composition of item 95.

194. A compound of any one of items 1 to 94 or a salt thereof for use in the treatment of a disease associated with a cysteine protease activity.

195. A composition for use in tire treatment of a disease associated with a cysteine protease activity comprising a compound of any one of items 1 to 94 or a salt thereof, and a pharmaceutically acceptable excipient.

196. Use of a compound of any one of items 1 to 94 or a salt thereof in the manufacture of a medicament for the treatment of a disease associated with a cysteine protease activity.

197. Use of a composition of item 95 in the manufacture of a medicament for the treatment of a disease associated with a cysteine protease activity.

198. The method, use, compound for use or composition for use according to any one of items 179 to 197, wherein the disease is selected from the group consisting of celiac disease, a gastrointestinal motility disorder, pain, itch, a skin disorder, diet-induced obesity, a metabolic disorder, asthma, rheumatoid arthritis, periodontitis, an inflammatory disease, a functional GI disorder, a cancer, a fibrotic disease, a metabolic dysfunction, a neurological disease, and a neurodegenerative disease.

199. The method, use, compound for use or composition for use according to item 198, wherein the functional GI disorder is selected from the group consisting of irritable bowel syndrome, functional chest pain, functional dyspepsia, nausea and vomiting disorders, functional constipation, functional diarrhea, fecal incontinence, functional anorectal pain, and functional defecation disorders.

200. The method, use, compound for use or composition for use according to item 199, wherein the functional GI disorder is irritable bowel syndrome.

201. The method, use, compound for use or composition for use according to any one of items 179 to 198, wherein the disease is selected from the group consisting of cancer, an inflammatory disease and a neurodegenerative disease.

202. The method, use, compound for use or composition for use according to any one of items 179 to 198, wherein the disease is cancer selected from the group consisting of breast cancer, brain cancer, bone marrow cancer, pancreatic cancer, lung cancer, prostate cancer, liver cancer, oral cancer, colorectal cancer and gastric cancer.

203. The method, use, compound for use or composition for use according to any one of items 179 to 198, wherein the disease is an inflammatory disease selected from the group consisting of an inflammatory GI disorder, pancreatitis, and an infection.

204. The method, use, compound for use or composition for use according to item 203, wherein the inflammatory GI disorder is selected from the group consisting of an inflammatory bowel disease, infectious diarrhea, mesenteric ischaemia, diverticulitis and necrotizing enterocolitis (NEC).

205. The method, use, compound for use or composition for use according to item 204, wherein the inflammatory bowel disease is selected from the group consisting of ulcerative colitis, Crohn’s disease, diversion colitis, indeterminate colitis and pouchitis, microscopic colitis, immuno-oncology colitis, chemotherapy/radiation colitis, Graft versus Host Disease (GvHD) colitis, acute colitis, Behcet’s disease, collagenous colitis, and lymphocytic colitis.

206. The method, use, compound for use or composition for use according to item 204, wherein the inflammatory bowel disease is ulcerative colitis or Crohn’s disease.

207. The method, use, compound for use or composition for use according to any one of items 179 to 198, wherein the disease is a neurodegenerative disease selected from the group consisting of Alzheimer’s disease, multiple sclerosis, and neuropathic pain.

208. The method, use, compound for use or composition for use according to any one of items 179 to 207, wherein the disease is a disease associated with cathepsin X activity.

209. A process for preparing a compound bearing a chloromethylketone moiety comprising reacting a compound bearing a sulfoxonium ylide moiety to yield the compound bearing the chloromethylketone moiety.

210. A process for preparing an activity-based probe compound bearing an acyloxymethylketone moiety or a phenoxymethylketone moiety as warhead, comprising
(i) preparing an intermediate compound bearing a chloromethylketone moiety by reacting a compound bearing a sulfoxonium ylide moiety to yield the compound bearing the chloromethylketone moiety; and
(ii) further processing the compound bearing the chloromethylketone moiety to yield said activity-based probe compound.

211. The process of item 209 or 210, wherein the compound bearing the sulfoxonium ylide moiety is reacted with hydrochloric acid to yield the compound bearing the chloromethylketone moiety.

212. The process of any one of items 209 to 211, wherein the sulfoxonium ylide moiety has the formula (IV)

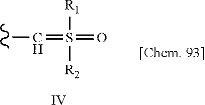

wherein
R₁ is selected from the group consisting of (C₁-C₈) alkyl, (C₁-C₈) hydroxyalkyl, (C₁-C₈) haloalkyl, (C₃-C₈) cycloalkyl, (C₂-C₈) alkenyl and (C₂-C₈ alkynyl; and
R₂ is selected from the group consisting of (C₁-C₈) alkyl, (C₁-C₈) hydroxyalkyl, (C₁-C₈ haloalkyl, (C₃-C₈) cycloalkyl, (C₂-C₈) alkenyl and (C₂-C₈) alkynyl.

213. The process of item 212, wherein R₁ is (C₁-C₈) alkyl, and R₂ is (C₁-C₈) alkyl.

214. The process of item 212, wherein R₁ is methyl and R₂ is methyl.

215. A compound selected from the group of formulas consisting of:

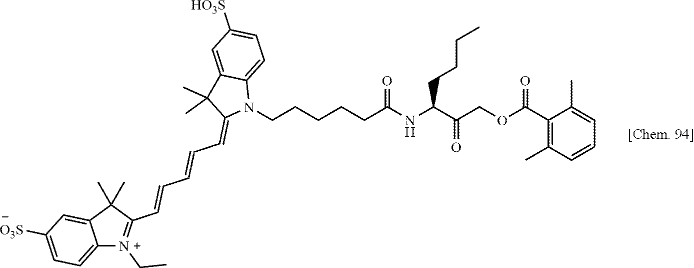

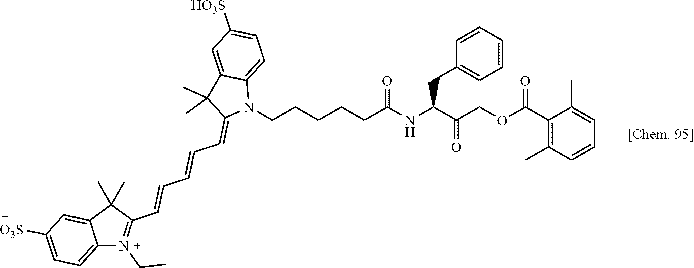

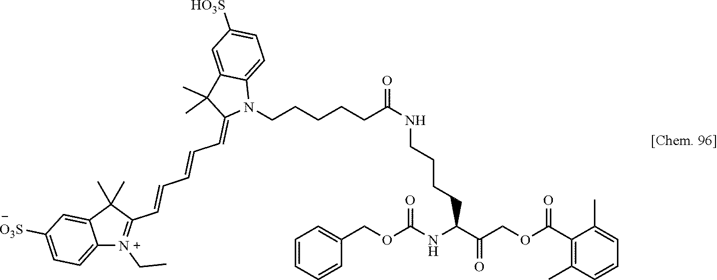

or a salt thereof.

1. A compound of formula I 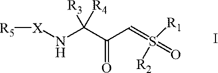
or a salt thereof, wherein
R₁ is selected from the group consisting of (C₁-C₈alkyl, (C₁-C₈) hydroxyalkyl, (C₁-C₈) haloalkyl, (C₃-C₈) cycloalkyl, (C₂-C₈) alkenyl and (C₂-C₈) alkynyl;
R₂ is selected from the group consisting of (C₁-C₈alkyl, (C₁-C₈) hydroxyalkyl, (C₁-C₈) haloalkyl, (C₃-C₈) cycloalkyl, (C₂-C₈) alkenyl and (C₂-C₈) alkynyl;
R₃ is the sidechain of an alpha amino acid;
R₄ is selected from the group consisting of hydrogen and (C₁-C₄) alkyl;
R₅ is selected from the group consisting of a detectable element, an amine protecting group, (C₁-C₈alkyl, (C₁-C₈) hydroxyalkyl, (C₁-C₈) haloalkyl, (C₃-C₈)cycloalkyl, (C₂-C₈) alkenyl, (C₂-C₈) alkynyl, (C₁-C₈) alkylcarbonyl, (C₁-C₈) hydroxyalkylcarbonyl, (C₁-C₈) haloalkylcarbonyl, (C₃-C₈) cycloalkylcarbonyl, (C₁-C₈) alkyloxycarbonyl, benzyloxycarbonyl, and hydrogen;
X is
(i) a bond; or
(ii) a biradical moiety of formula II or III which is connected to the R₅ substituent via the amino group 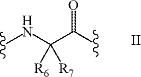 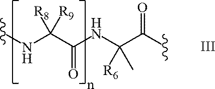
wherein
R₆ is the sidechain of an alpha amino acid;
R₇ is selected from the group consisting of hydrogen and (C₁-C₄) alkyl;
Rs is the sidechain of an alpha amino acid;
R₉ is selected from the group consisting of hydrogen and (C₁-C₄) alkyl; and
n is 1, 2, 3, or 4.

2. The compound of claim 1,
wherein R₃ is the sidechain of a natural alpha amino acid, or a structural isomer, homologue and/or structural analogue of said sidechain,
wherein said sidechain or structural isomer, homologue and/or structural analogue thereof is optionally substituted by an amine protecting group or a detectable element and optionally further substituted by one or more, same or different substituents R₃ₓ, wherein
R_{3X} is selected from the group consisting of hydroxy, halogen, (C₁-C₄) alkyl, (C₁-C₄) hydroxyalkyl, (C₁-C₄) haloalkyl, (C₁-C₄) alkoxy, and (C₁-C₄) haloalkoxy; or
wherein R₃ is the sidechain of a proteinogenic alpha amino acid, or a structural isomer, homologue and/or structural analogue of said sidechain,
wherein said sidechain or structural isomer, homologue and/or structural analogue thereof is optionally substituted by an amine protecting group or a detectable element and optionally further substituted by one or more, same or different substituents R_{3X}, wherein
R₃ₓ is selected from the group consisting of hydroxy, halogen, (C₁-C₄) alkyl, (C₁-C₄) hydroxyalkyl, (C₁-C₄) haloalkyl, (C₁-C₄) alkoxy, and (C₁-C₄) haloalkoxy; or
wherein R₃ is the sidechain of an alpha amino acid selected from the group consisting of glycine, alanine, alpha-aminobutyric acid, valine, norvaline, leucine, isoleucine, norleucine, homonorleucine, methionine, ethionine, phenylalanine, tyrosine, levodopa, tryptophan, cysteine, homocysteine, selenocysteine, selenohomocysteine, selenomethionine, selenoethionine, lysine, histidine, arginine, omithine, aspartic acid, glutamic acid, serine, homoserine, O-methyl-homoserine, O-ethyl-homoserine, threonine, asparagine, and glutamine, or a structural isomer, homologue and/or structural analogue of said sidechain,
wherein said sidechain or structural isomer, homologue and/or structural analogue thereof is optionally substituted by an amine protecting group or a detectable element and optionally further substituted by one or more, same or different substituents R_{3X}, wherein
R₃ₓ is selected from the group consisting of hydroxy, halogen, (C₁-C₄) alkyl, (C₁-C₄) hydroxyalkyl, (C₁-C₄) haloalkyl, (C₁-C₄) alkoxy, and (C₁-C₄) haloalkoxy; or
wherein R₃ is the sidechain of an alpha amino acid selected from the group consisting of glycine, alanine, alpha-aminobutyric acid, valine, norvaline, leucine, isoleucine, norleucine, homonorleucine, methionine, ethionine, phenylalanine, tyrosine, levodopa, tryptophan, cysteine, homocysteine, selenocysteine, selenohomocysteine, selenomethionine, selenoethionine, histidine, arginine, ornithine, aspartic acid, glutamic acid, serine, homoserine, O-methyl-homoserine, O-ethyl-homoserine, threonine, asparagine, and glutamine, or a structural isomer or homologue of said sidechain,
wherein said sidechain or structural isomer or homologue thereof is optionally substituted by an amine protecting group or a detectable element and optionally further substituted by one or more, same or different substituents R₃ₓ, wherein
R₃ₓ is selected from the group consisting of hydroxy, halogen, (C₁-C₄) alkyl, (C₁-C₄) hydroxyalkyl, (C₁-C₄) haloalkyl, (C₁-C₄) alkoxy, and (C₁-C₄) haloalkoxy;
optionally wherein the alpha amino acid is selected from the group consisting of alanine, alpha-aminobutyric acid, valine, norvaline, leucine, isoleucine, norleucine, homonorleucine, phenylalanine, and tryptophan.

3. The compound of claim 1, wherein
(i) R₃ is selected from the group consisting of hydrogen, (C₁-C₈) alkyl, (C₁-C₈) hydroxyalkyl, (C₁-C₈) haloalkyl, (C₃-C₈)cycloalkyl, (C₂-C₈) alkenyl, (C₂-C₈) alkynyl, (C₆-C₁₀) arylmethyl, (C₃-C₉) heteroarylmethyl, and -CH₂CH₂CH₂CH₂N(R₃ₐ)(R_{3b}); wherein
R₃ₐ is selected from the group consisting of a detectable element, an amine protecting group, hydrogen, and (C₁-C₈alkyl; and
R_{3b} is selected from the group consisting of hydrogen and (C₁-C₄) alky or
(ii) R₃ is -CH₂CH₂CH₂CH₂N(R₃ₐ)(R_{3b}); wherein
R₃ₐ is selected from the group consisting of a detectable element, an amine protecting group, hydrogen, and (C₁-C₈) alkyl; and
R_{3b} is selected from the group consisting of hydrogen and (C₁-C₄) alkyl;
optionally wherein if X is a bond, R₃ₐ is not an amine protecting group, hydrogen, or (C₁-C₈) alkyl.

4-7. (canceled)

8. The compound of claim 1,
wherein the compound is defined by one or more of the following (i) to (iv):
(i) R₁ is (C₁-C₈) alkyl, optionally wherein R₁ is methyl;
(ii) R₂ is (C₁-C₈) alkyl, optionally wherein R₂ is methyl;
(iii) R₄ is hydrogen;
(iv) R₅ is selected from the group consisting of a detectable element, an amine protecting group, and hydrogen,
(v) R₆ is the sidechain of an alpha amino acid selected from the group consisting of glycine, alanine, alpha-aminobutyric acid, valine, norvaline, leucine, isoleucine, norleucine, homonorleucine, methionine, ethionine, phenylalanine, tyrosine, levodopa, tryptophan, cysteine, homocysteine, selenocysteine, selenohomocysteine, selenomethionine, selenoethionine, lysine, histidine, arginine, ornithine, aspartic acid, glutamic acid, serine, homoserine, O-methyl-homoserine, O-ethyl-homoserine, threonine, asparagine, and glutamine, or a structural isomer, homologue and/or structural analogue of said sidechain,
wherein said sidechain or structural isomer, homologue and/or structural analogue thereof is optionally substituted by an amine protecting group or a detectable element and optionally further substituted by one or more, same or different substituents R₆ₓ, wherein R₆ₓ is selected from the group consisting of hydroxy, halogen, (C₁-C₄) alkyl, (C₁-C₄) hydroxyalkyl, (C₁-C₄) haloalkyl, (C₁-C₄) alkoxy, and (C₁-C₄) haloalkoxy;
(vi) R₇ is hydrogen;
(vii) R₈ is the sidechain of an alpha amino acid selected from the group consisting of glycine, alanine, alpha-aminobutyric acid, valine, norvaline, leucine, isoleucine, norleucine, homonorleucine, methionine, ethionine, phenylalanine, tyrosine, levodopa, tryptophan, cysteine, homocysteine, selenocysteine, selenohomocysteine, selenomethionine, selenoethionine, lysine, histidine, arginine, ornithine, aspartic acid, glutamic acid, serine, homoserine, O-methyl-homoserine, O-ethyl-homoserine, threonine, asparagine, and glutamine, or a structural isomer, homologue and/or structural analogue of said sidechain,
wherein said sidechain or structural isomer, homologue and/or structural analogue thereof is optionally substituted by an amine protecting group or a detectable element and optionally further substituted by one or more, same or different substituents R₈ₓ, wherein
R₈ₓ is selected from the group consisting of hydroxy, halogen, (C₁-C₄) alkyl, (C₁-C₄) hydroxyalkyl, (C₁-C₄) haloalkyl, (C₁-C₄) alkoxy, and (C₁-C₄) haloalkoxy;
(viii) R₉ is hydrogen, and
(iv) the amine protecting group is selected from the group consisting of benzyloxycarbonyl (Cbz), 9-fluorenylmethyloxycarbonyl (Fmoc), tert-butyloxycarbonyl (Boc), allyloxycarbonyl (Alloc), p-toluenesulfonyl (Tos), 2,2,5,7,8-pentamethylchroman-6-sulfonyl (Pmc), 2,2,4,6,7-pentamethyl-2,3-dihydrobenzofuran-5-sulfonyl (Pbf), mesityl-2-sulfonyl (Mts), 4-methoxy-2,3,6-trimethylphenylsulfonyl (Mtr), acetamido, and phthalimido;
optionally wherein the amine protecting group is benzyloxycarbonyl.

9-11. (canceled)

12. The compound of claim 1, wherein X is a bond and/or wherein n is 1.

13. The compound of claim 1, wherein the detectable element is selected from the group consisting of a fluorescent label, a biotin label, a radiolabel, a chelator, and a bioorthogonal ligation handle,
and optionally wherein the detectable element is a fluorescent label;
optionally wherein the fluorescent label is selected from the group consisting of a fluorescein, an Oregon green, a bora-diaza-indecene dye, a rhodamine dye, a benzopyrillium dye, a coumarin dye, a cyanine label or a benzoindole label, and
optionally wherein the fluorescent label is a cyanine label optionally having a formula selected from:
(i) the following group of formulas: 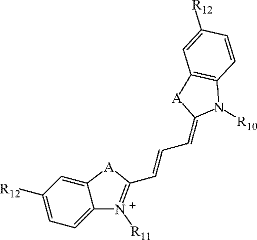 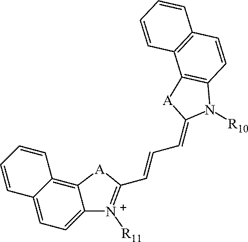 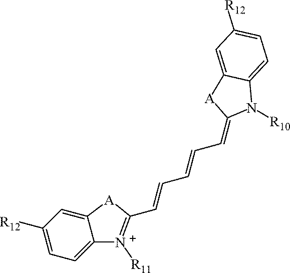 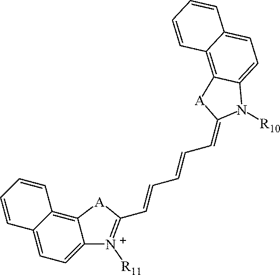 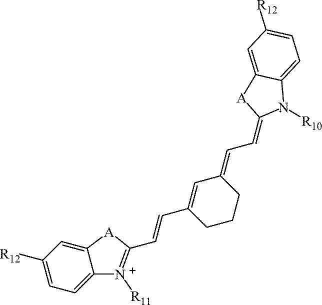 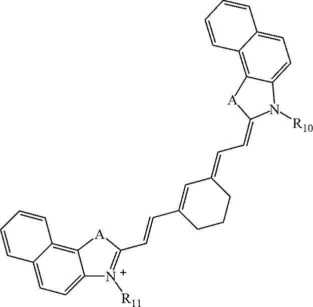
wherein in each of the above formulas,
A is selected from the group consisting of CH₂, C(CH₃)₂, C(C₂H₅)₂, NH, N(CH₃), N(C₂H₅), O, S, and Se;
R₁₀ is selected from the group consisting of $-(CH₂)ₚ-C(=O)-& and $-(CH₂)_{q}-C(=O)-NH-[CH₂CH₂O]ᵣ-CH₂CH₂-C(=O)-&; wherein
p is 2, 3, 4, 5, 6, 7, or 8;
q is 2, 3, 4, 5, 6, 7, or 8_{:}
r is 2, 3, 4, 5, 6, 7, or 8;
$ represents the point of connection to the nitrogen atom of the cyanine moiety: and & represents the point of connection to the remainder of the molecule:
R₁₁ is selected from the group consisting of (C₁-C₈)alkyl, and (C₆-C₁₀)aryl; and
R₁₂ is H or a sulfo group, or
(ii) the following group of formulas: 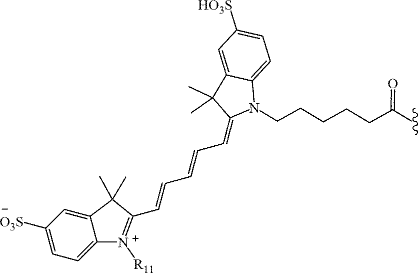 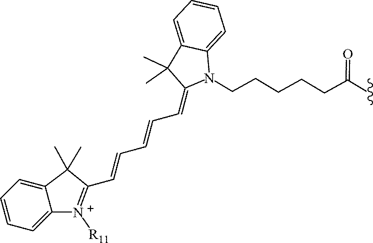 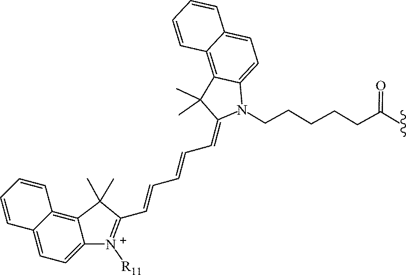
wherein in each of the above formulas,
the curled line represents the point of connection to the remainder of the molecule;
and R₁₁ is selected from the group consisting of (C₁-C₈)alkyl, and (C₆-C₁₀aryl; or
wherein the fluorescent label is a cyanine label having the formula 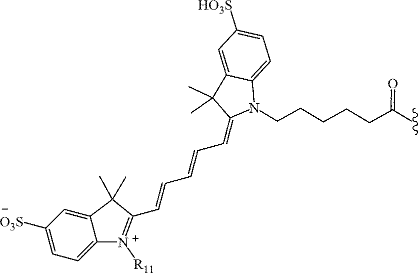
wherein the curled line represents the point of connection to the remainder of the molecule; and R₁₁ is methyl or ethyl.

14-16. (canceled)

17. The compound of claim 1,
wherein the compound comprises at least one detectable element; or
wherein the compound comprises one, two or three detectable elements; or
wherein the compound comprises one detectable element.

18. The compound of claim 1, wherein R₅ is a detectable element.

19. The compound of claim 1, wherein R₃ bears a detectable element; optionally
wherein R₃ is the sidechain of lysine, or a structural isomer, homologue and/or structural analogue of said sidechain,
wherein said sidechain or structural isomer, homologue and/or structural analogue thereof is substituted by a detectable element and optionally further substituted by one or more, same or different substituents R₃ₓ, wherein R₃ₓ is selected from the group consisting of hydroxy, halogen, (C₁-C₄) alkyl, (C₁-C₄) hydroxyalkyl, (C₁-C₄) haloalkyl, (C₁-C₄) alkoxy, and (C₁-C₄) haloalkoxy; or
wherein R₃ is -CH₂CH₂CH₂CH₂N(R₃ₐ)(R_{3b}); wherein
R₃ₐ is a detectable element; and
R_{3b} is selected from the group consisting of hydrogen and (C₁-C₄) alkyl.

20. The compound of claim 19,
wherein R₅ is selected from the group consisting of an amine protecting group, (C₁-C₈) alkyl, (C₁-C₈) hydroxyalkyl, (C₁-C₈) haloalkyl, (C₃-C₈) cycloalkyl, (C₂-C₈) alkenyl, (C₂-C₈) alkynyl, (C₁-C₈) alkylcarbonyl, (C₁-C₈) hydroxyalkylcarbonyl, (C₁-C₈) haloalkylcarbonyl, (C₃-C₈) cycloalkylcarbonyl, (C₁-C₈) alkyloxycarbonyl, benzyloxycarbonyl, and hydrogen; or
wherein R₅ is an amine protecting group.

21. The compound of claim 1, which is a compound selected from the group of formulas consisting of: 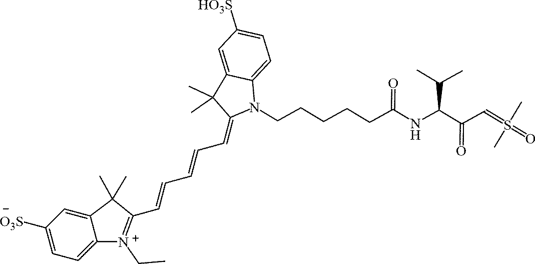 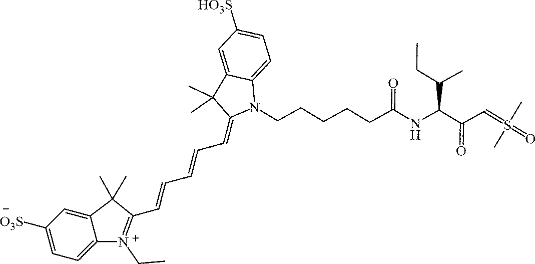 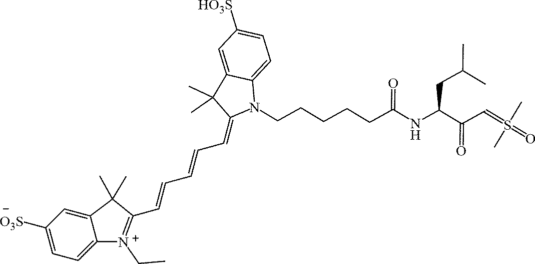 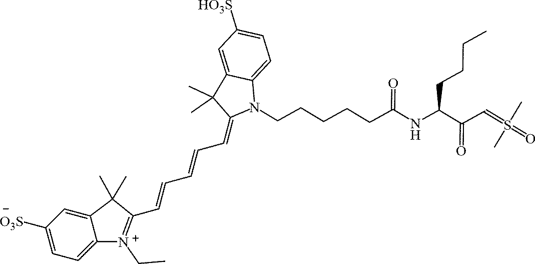 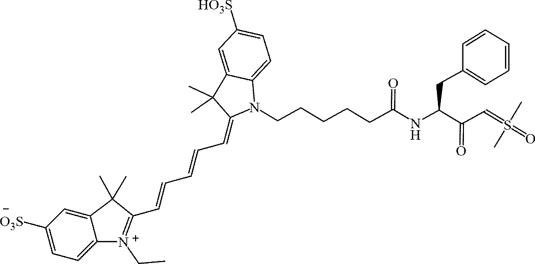 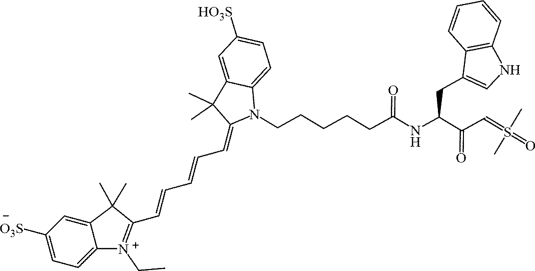 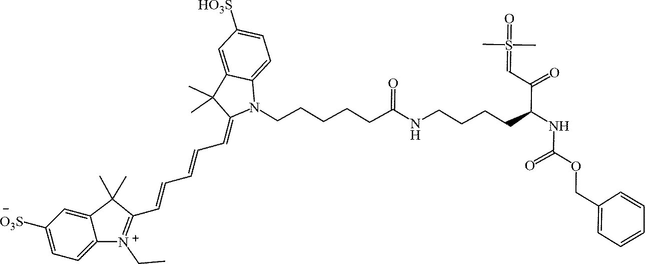 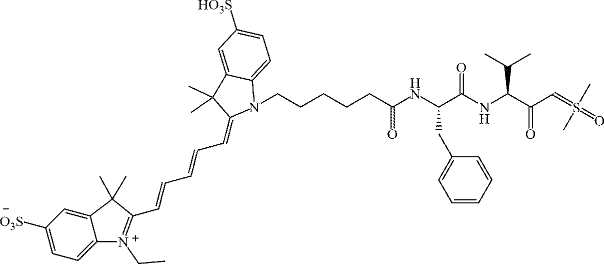
or a salt thereof; optionally
wherein the compound is a compound of formula: 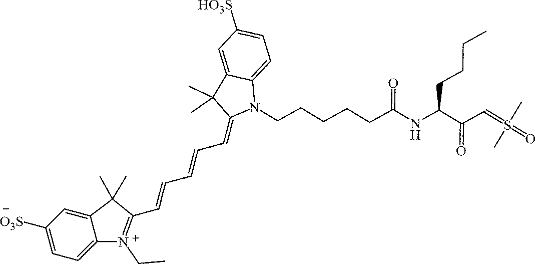
or a salt thereof.

22. A composition comprising a compound of claim 1 or a salt thereof, and an excipient,
and optionally wherein the composition comprises a compound of claim 17 or a salt thereof, and an excipient.

23. (canceled)

24. A method of detecting cysteine protease activity in a biological sample obtained from a subject comprising
(1) contacting the biological sample in vitro with an activity-based probe compound comprising a sulfoxonium ylide moiety as warhead, and
(2) subsequently analyzing the biological sample comprising measuring a detectable signal; optionally wherein the sulfoxonium ylide moiety has the formula (IV) 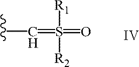
wherein
R₁ is selected from the group consisting of (C₁-C₈) alkyl, (C₁-C₈) hydroxyalkyl, (C₁-C₈) haloalkyl, (C₃-C₈) cycloalkyl, (C₂-C₈) alkenyl and (C₂-C₈) alkynyl; and
R₂ is selected from the group consisting of (C₁-C₈) alkyl, (C₁-C₈) hydroxyalkyl, (C₁-C₈) haloalkyl, (C₃-C₈) cycloalkyl, (C₂-C₈) alkenyl and (C₂-C₈) alkynyl.

25. The method of claim 24, wherein the activity-based probe compound is a compound of claim 17.

26-27. (canceled)

28. The method of claim 24,
wherein said compound comprises a detectable element in the form of a fluorescent label; or
wherein said compound comprises a detectable element in the form of a bioorthogonal ligation handle, and wherein step (2) comprises secondary labeling by click-chemistry to apply a fluorescent label prior to performing the at least one analytical method; or
wherein said compound comprises a detectable element in the form of biotin, and wherein step (2) comprises secondary labeling with fluorescently tagged streptavidin or secondary labeling with a fluorescently tagged antibody specific for biotin, prior to performing the at least one analytical method.

29. (canceled)

30. The method of claim 24,
wherein the biological sample is selected from the group consisting of cells, cell lysates, tissue samples, tissue lysates and bodily fluids; and/or
wherein the biological sample is obtained from a human subject,
and optionally
wherein the biological sample is a cell lysate or a tissue lysate; or
wherein the biological sample is a cleared cell lysate or a cleared tissue lysate; or
wherein the biological sample is live cells; or
wherein the live cells are lysed and cleared between step (1) and step (2).

31. (canceled)

32. A method of detecting cysteine protease activity comprising
(1) administering to a subject an activity-based probe compound comprising a sulfoxonium ylide moiety as warhead,
(2) subsequently obtaining a biological sample from the subject; and
(3) subsequently analyzing the biological sample comprising measuring a detectable signal; optionally wherein the sulfoxonium ylide moiety has the formula (IV) 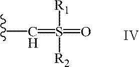
wherein
R₁ is selected from the group consisting of (C₁-C₈) alkyl, (C₁-C₈) hydroxyalkyl, (C₁-C₈) haloalkyl, (C₃-C₈) cycloalkyl, (C₂-C₈) alkenyl and (C₂-C₈) alkynyl; and
R₂ is selected from the group consisting of (C₁-C₈) alkyl, (C₁-C₈) hydroxyalkyl, (C₁-C₈) haloalkyl, (C₃-C₈) cycloalkyl, (C₂-C₈) alkenyl and (C₂-C₈) alkynyl.

33. The method of claim 32, wherein the activity-based probe compound is a compound of claim 17.

34-35. (canceled)

36. The method of claim 32,
wherein said compound comprises a detectable element in the form of a fluorescent label; or
wherein said compound comprises a detectable element in the form of a bioorthogonal ligation handle, and wherein step (3) comprises secondary labeling by click-chemistry to apply a fluorescent label prior to performing the at least one analytical method; or
wherein said compound comprises a detectable element in the form of biotin, and wherein step (3) comprises secondary labeling with fluorescently tagged streptavidin or secondary labeling with a fluorescently tagged antibody specific for biotin, prior to performing the at least one analytical method.

37. (canceled)

38. The method of claim 32,
wherein the biological sample is selected from the group consisting of cells, cell lysates, tissue samples, tissue lysates and bodily fluids; optionally
wherein the biological sample is a cell lysate or a tissue lysate; or
wherein the biological sample is a cleared cell lysate or a cleared tissue lysate, and/or
wherein the subject is a human subject.

39. (canceled)

40. An in vivo method of detecting cysteine protease activity in a subject comprising
(1) administering to the subject an activity-based probe compound comprising a sulfoxonium ylide moiety as warhead, and
(2) subsequently examining the subject comprising measuring a detectable signal; optionally wherein the sulfoxonium ylide moiety has the formula (IV) 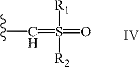
wherein
R₁ is selected from the group consisting of (C₁-C₈) alkyl, (C₁-C₈) hydroxyalkyl, (C₁-C₈) haloalkyl, (C₃-C₈) cycloalkyl, (C₂-C₈) alkenyl and (C₂-C₈) alkynyl; and R₂ is selected from the group consisting of (C₁-C₈) alkyl, (C₁-C₈) hydroxyalkyl, (C₁-C₈) haloalkyl, (C₃-C₈) cycloalkyl, (C₂-C₈) alkenyl and (C₂-C₈) alkynyl.

41. The method of claim 40, wherein the activity-based probe compound is a compound of claim 17.

42. The method of claim 40,
wherein the detectable signal is measured by in vivo optical imaging, radiography, or positron emission tomography; and/or
wherein the subject is a human subject.

43-45. (canceled)

46. A method of diagnosing a disease associated with a cysteine protease activity in a subject comprising
(1) contacting a biological sample obtained from the subject in vitro with a compound of claim 17 or a salt thereof, and
(2) subsequently analyzing the biological sample comprising measuring a detectable signal.

47-51. (canceled)

52. The method according to claim 46, wherein the disease is a disease associated with cathepsin X activity.

53. The method of claim 24,
wherein the cysteine protease is cysteine cathepsin, and optionally a human cysteine cathepsin, or
wherein the cysteine protease is cathepsin X, and optionally human cathepsin X, and/or
wherein cathepsin X activity is detected and cathepsin B activity and/or cathepsin L activity are not detected; or
wherein cathepsin X activity and cathepsin S activity are detected and cathepsin B activity and/or cathepsin L activity are not detected.

54. The method of claim 32,
wherein the cysteine protease is cysteine cathepsin, and optionally a human cysteine cathepsin, or
wherein the cysteine protease is cathepsin X, and optionally human cathepsin X, and/or
wherein cathepsin X activity is detected and cathepsin B activity and/or cathepsin L activity are not detected; or
wherein cathepsin X activity and cathepsin S activity are detected and cathepsin B activity and/or cathepsin L activity are not detected.

55. The method of claim 40,
wherein the cysteine protease is cysteine cathepsin, and optionally a human cysteine cathepsin, or
wherein the cysteine protease is cathepsin X, and optionally human cathepsin X, and/or
wherein cathepsin X activity is detected and cathepsin B activity and/or cathepsin L activity are not detected; or
wherein cathepsin X activity and cathepsin S activity are detected and cathepsin B activity and/or cathepsin L activity are not detected.
